(19) United States
(12) Patent Application Publication
(10) Pub. No.: US 2024/0124614 A1
(43) Pub. Date: Apr. 18, 2024

(54) ANTIGEN-BINDING MOLECULES WITH IMPROVED CYTOSOL-PENETRATING ACTIVITY

(71) Applicant: Chugai Seiyaku Kabushiki Kaisha, Tokyo (JP)

(72) Inventors: Yuri IKAWA, Shizuoka (JP); Nasa SAVORY, Singapore (SG)

(21) Appl. No.: 18/269,099

(22) PCT Filed: Dec. 22, 2021
(86) PCT No.: PCT/JP2021/047478

§ 371 Date: Jun. 22, 2023

(30) Foreign Application Priority Data
Dec. 23, 2020 (JP) 2020-213219

(51) Int. Cl. C07K 16/46

(52) U.S. Cl. CPC C07K 16/468; C07K 2317/31; C07K 2317/565; C07K 2317/567; C07K 2317/77

(57) ABSTRACT

The present invention provides an antigen-binding molecule (e.g. antibody) with improved cytosol-penetrating ability, the molecule comprising an amino acid substitutions on at least one selected from the group consisting of CDRL1, CDRL2 and CDRL3 in a light-chain variable region, or an antigen-binding fragment (antibody fragment) thereof; and pharmaceutical compositions comprising the antigen-binding molecule; methods for delivering the antigen-binding molecule specifically into the cytosol of a target cell; methods of removing, suppressing, or activating a cytosolic antigen in a target cell-specific manner by using the antigen-binding molecule; and pharmaceutical compositions for diagnosing, preventing, or treating a disease in a subject comprising the antigen-binding molecule.

### TECHNICAL FIELD

The present disclosure relates to antigen-binding molecules (e.g. antibodies) having improved cytosol-penetrating ability, and a cytosol-penetrating antigen-binding molecule (e.g. antibody) comprising an amino acid mutation on at least one selected from the group consisting of CDRL1, CDRL2 and CDRL3 in a light chain variable region, or an antigen-binding fragment (antibody fragment) thereof; and pharmaceutical compositions comprising the antigen-binding molecule; methods for delivering the antigen-binding molecule specifically into the cytosol of a target cell; methods of removing, suppressing, or activating a cytosolic antigen in a target cell-specific manner by using the antigen-binding molecule; and pharmaceutical compositions for diagnosing, preventing, or treating a disease in a subject comprising the antigen-binding molecule.

### BACKGROUND ART

Antibodies are proteins which specifically bind to an antigen with high affinity. It is known that various molecules ranging from low-molecular weight compounds to proteins can be antigens. Since the technique for producing monoclonal antibodies was developed, antibody modification techniques have advanced, making it easy to obtain antibodies that recognize a particular molecule. Antibodies are drawing attention as pharmaceuticals because they are highly stable in blood plasma and have less side effects. Not only do antibodies bind to an antigen and exhibit agonistic or antagonistic effects, but they also induce cytotoxic activity mediated by effector cells (also referred to as effector functions) including ADCC (Antibody Dependent Cytotoxicity), ADCP (Antibody Dependent Cell Phagocytosis), and CDC (Complement Dependent Cytotoxicity). Taking advantage of these antibody functions, pharmaceuticals for cancer, immune diseases, chronic diseases, infections, etc. have been developed (Nat Rev Drug Discov. 2018 Mar.; 17(3):197-223. (NPL 1)).

Meanwhile, antigens targeted by antibody pharmaceuticals are limited to antigens on cell membrane or antigens outside cells, as full-length IgG molecules are of high molecular weight (about 150 kDa) and generally do not exhibit cell permeability. Thus, intrabodies (antibodies or antibody fragments expressed inside cells), antibody-CPP complexes produced by fusion with a cell penetrating peptide (CPP), the protein transfection method, etc. were developed and reported as technologies to allow antibodies to act on intracellular antigens (MAbs. 2011 Jan.-Feb.;3(1):3-16. (NPL 2)).

Some naturally occurring full length antibodies are known to have cell-penetrating ability. For example, anti-DNA autoantibodies identified from systemic lupus erythematosus (SLE) patients and MRL-mpj/lpr lupus model mice were reported to show cell-penetrating ability (Sci Rep. 2015 Jul. 9; 5:12022. (NPL 3), Mol Immunol. 2015 October; 67(2 Pt B):377-87. (NPL 4)). Some of the anti-DNA antibodies also show DNA hydrolysis activity or DNA repair-inhibiting activity and exhibit cytotoxicity, in addition to the cell-penetrating ability (WO 2012135831 (PTL 1), Sci Rep. 2014 Aug. 5; 4:5958 (NPL 5)). There is also a report on an antibody prepared by humanizing an antibody isolated from an SLE model mouse that has ability to escape from endosomes into the cytosol, and combining it with a heavy chain variable region that can bind to activated Ras (WO2016013871A1 (PTL 2)).

### CITATION LIST

### Patent Literature

[PTL 1] WO2012/135831
[PTL 2] WO2016/013871A1

### Non-Patent Literature

[NPL 1] Nat Rev Drug Discov. 2018 Mar.; 17(3):197-223.
[NPL 2] MAbs. 2011 Jan.-Feb.; 3(1):3-16.
[NPL 3] Sci Rep. 2015 Jul. 9; 5:12022.
[NPL 4] Mol Immunol. 2015 October; 67(2 Pt B):377-87.
[NPL 5] Sci Rep. 2014 Aug. 5; 4:5958

### SUMMARY OF INVENTION

### Technical Problem

Most antibody therapeutics developed to date cannot target cytosol molecules as they only target surface exposed or secreted molecules, and therefore development of a method of delivering the antibody through the cell membrane is needed. In one non-limiting embodiment, an objective of the present invention is to provide an antigen-binding molecule (e.g. antibody) that is to be delivered specifically to the cytosol of a target cell efficiently, a pharmaceutical composition comprising the antigen-binding molecule, a method of using the antigen-binding molecule, a method of producing the antigen-binding molecule, and a method for delivering an antigen-binding molecule specifically into the cytosol of a target cell.

### Solutions to Problem

The present invention has been derived by the above-mentioned needs, and provides antigen-binding molecules (e.g. antibodies) with improved cytoplasmic permeability, and confirming that the antigen-binding molecules or antibodies have improved permeability to the cytoplasm. In one non-limiting embodiment, the present invention relates to an antigen-binding molecule (e.g. antibody) or fragment thereof with enhanced cytosol-penetrating ability or activity, comprising a light chain variable region comprising CDRL1, CDRL2, and CDRL3 of the amino acid sequence set forth in SEQ ID NO: 4 and comprising one or more amino acid substitutions in at least one selected from the CDRL1, CDRL2, and CDRL3, preferably from the CDRL1 and CDRL3.

In one non-limiting embodiment, the present inventors have identified antigen-binding molecules (antibodies) with improved or enhanced cytosol-penetrating ability or activity while additionally having superior properties in terms of (1) antibody expression level, (2) reduced extracellular matrix (ECM)-binding profile (which improves the half-life or pharmacokinetics in vivo), and/or (3) good physicochemical property indicated by lower aggregation tendency.

In one aspect, the present disclosure provides:
[1] A cytosol-penetrating antibody or antigen-binding fragment thereof, comprising a light chain variable region comprising CDRL1, CDRL2, and CDRL3 of the amino acid sequence set forth in SEQ ID NO: 4, wherein the light chain variable region comprises one or more amino acid substitutions in at least one selected from the CDRL1, CDRL2, and CDRL3.
[2] The cytosol-penetrating antibody or antigen-binding fragment thereof of [1], wherein said light chain variable region comprises one or more amino acid substitutions at Kabat numbering positions 24 to 34 and 89 to 97 in the amino acid sequence set forth in SEQ ID NO: 4.
[3] The cytosol-penetrating antibody or antigen-binding fragment thereof of [2], wherein said light chain variable region comprises one or more amino acid substitutions at Kabat numbering positions 24 to 32 and 93 to 97 in amino acid sequence set forth in SEQ ID NO: 4.
[4] The cytosol-penetrating antibody or antigen-binding fragment thereof of [3], wherein said one or more amino acid substitutions are selected from the group consisting of (position according to Kabat numbering):
(a) substitution of amino acid K at position 24 with A, D, E, F, G, H, I, L, N, Q, R, S, T, V or Y;
(b) substitution of amino acid S at position 25 with A, D, E, F, I, M, N, T, V or Y;
(c) substitution of amino acid S at position 26 with A, D, E, F, G, H, I, K, M, N, P, R, T or V;
(d) substitution of amino acid Q at position 27 with A, D, E, F, H, I, L, M, S, or Y;
(e) substitution of amino acid S at position 27a with D;
(f) substitution of amino acid L at position 27b with I, P, Q, T or V;
(g) substitution of amino acid F at position 27c with A, D, E, I, M, N, S, W or Y;
(h) substitution of amino acid N at position 27d with A, D, E, F, H, N or Q;
(i) substitution of amino acid S at position 27e with A, D, E, I, L, M, N, P, Q, T, V or Y;
(j) substitution of amino acid R at position 27f with A, E, F, G, H, I, K, L, M, N, Q, S, T, V, W or Y;
(k) substitution of amino acid T at position 28 with E, H, I, Q, S, V, W or Y;
(l) substitution of amino acid R at position 29 with A, D, E, F, G, H, I, K, L, M, N, P, Q, S, T, V, W or Y;
(m) substitution of amino acid K at position 30 with A, D, E, F, H, I, L, M, N, P, Q, S, T, V, W or Y;
(n) substitution of amino acid N at position 31 with D, M, S or T;
(o) substitution of amino acid Y at position 32 with D, E, G, H or Q;
(p) substitution of amino acid Q at position 89 with H, I, T or Y;
(q) substitution of amino acid Y at position 91 with E, F or N;
(r) substitution of amino acid Y at position 93 with I, L, M, N, P, Q, S, T, V or W;
(s) substitution of amino acid H at position 94 with A, D, E, F, G, I, L, M, N, P, Q, R, S, T, V, W or Y;
(t) substitution of amino acid M at position 95 with A, D, E, F, G, H, I, K, L, N, P, Q, R, S, T, V, W or Y;
(u) substitution of amino acid Y at position 96 with A, D, E, F, G, H, K, M, N, P, Q, S, T or V; and
(v) substitution of amino acid T at position 97 with A, D, E, G, H, K, P, Q, R, S or V; in the amino acid sequence set forth in SEQ ID NO: 4.
   [4A] The cytosol-penetrating antibody or antigen-binding fragment thereof of [4], wherein each of the CDRL1 and CDRL3 in the light chain variable region comprises two or more amino acid substitutions selected from the group defined in [4](a) to (v).
   [5] The cytosol-penetrating antibody or antigen-binding fragment thereof of any one of [1] to [4A], wherein said amino acid substitutions enhance the cytosol-penetrating activity of the antibody or fragment thereof as compared to a reference antibody or fragment thereof without said substitutions and comprising a light chain variable region having the amino acid sequence set forth in SEQ ID NO: 4.
   [5A] The cytosol-penetrating antibody or antigen-binding fragment thereof of any one of [1] to [5], which exhibits at least 1%, 5%, 8%, 10%, 12% 15%, 18%, 20%, 25%, 30%, 35%, 40%, 50%, 60%, 70%, 80%, 90%, 100%, 200% or 500% or 1000% enhanced cytosol-penetrating activity compared to a reference antibody or fragment thereof without said substitutions and comprising a light chain variable region having the amino acid sequence set forth in SEQ ID NO: 4, preferably as determined using Split Nluc assay as described in Reference Example 1.
   [5B] The cytosol-penetrating antibody or antigen-binding fragment thereof of any one of [1] to [5A], wherein the cytosol-penetrating activity is mediated by penetration of the antibody or fragment thereof into cell membrane, and/or endosomal escape after penetration of the antibody or fragment thereof into cell membrane.
   [5C] The cytosol-penetrating antibody or antigen-binding fragment thereof of any one of [1] to [5B], which exhibits one or more properties relative to the reference antibody, wherein the one or more properties are selected from the group consisting of:
(a) increased antibody expression level relative to the reference antibody;
(b) reduced binding activity to extracellular matrix (ECM) relative to the reference antibody, preferably as determined by a method described in Reference Example 2; and
(c) improved physicochemical property relative to the reference antibody, preferably as indicated by higher monomer peak percentage as determined by size-exclusion chromatography (SEC) analysis.
   [6] The cytosol-penetrating antibody or antigen-binding fragment thereof of any one of [1] to [5C], wherein the light chain variable region comprises one, two, three or more amino acid substitutions selected from the group consisting of (position according to Kabat numbering):
(a) substitution of amino acid K at position 24 with N;
(b) substitution of amino acid Q at position 27 with E;
(c) substitution of amino acid S at position 27a with D;
(d) substitution of amino acid N at position 27d with D or E;
(e) substitution of amino acid S at position 27e with D or E;
(f) substitution of amino acid R at position 27f with D, H, K, L, S or V;
(g) substitution of amino acid T at position 28 with D;
(h) substitution of amino acid R at position 29 with E, G, M or S;
(i) substitution of amino acid K at position 30 with L or Q;
(j) substitution of amino acid Y at position 32 with E;
(k) substitution of amino acid Q at position 89 with H, I, T or Y;
(l) substitution of amino acid Y at position 91 with E, F or N;
(m) substitution of amino acid Y at position 93 with E;
(n) substitution of amino acid H at position 94 with E, P or Q;
(o) substitution of amino acid M at position 95 with D, N or P; and
(p) substitution of amino acid Y at position 96 with E or P;
in the amino acid sequence set forth in SEQ ID NO: 4.
   [6A] The cytosol-penetrating antibody or antigen-binding fragment thereof of [6], wherein each of the CDRL1 and CDRL3 in the light chain variable region comprises two or more amino acid substitutions selected from the group defined in [6](a) to (p).
   [7] The cytosol-penetrating antibody or antigen-binding fragment thereof of [6], wherein the light chain variable region comprises a combination of amino acid substitutions selected from the group consisting of (position according to Kabat numbering):
      (a) S27eD/R27fK;
      (b) S27eD/R27fH;
      (c) N27dE/S27eD;
      (d) S27eD/T28D;
      (e) S27eD/R29E;
      (f) S27eD/R29S;
      (g) K24N/S27eD;
      (h) N27dD/R27fK;
      (i) N27dE/R27fK;
      (j) R27fK/T28D;
      (k) R27fK/R29E;
      (l) R27fK/R29S;
      (m) K24N/R27fK;
      (n) K24N/N27dD;
      (o) N27dD/T28D;
      (p) N27dD/R29E;
      (q) N27dD/R29S;
      (r) S27eD/Q89H;
      (s) N27dD/Q89H;
      (t) R27fK/Q89H;
      (u) S27eD/Y91E;
      (v) N27dD/Y91E;
      (w) R27fK/Y91E;
      (x) Q89H/H94P;
      (y) Y91E/H94P;
      (z) H94P/M95N;
      (aa) H94P/M95D;
      (bb) H94P/M95P;
      (cc) H94E/Y96P;
      (dd) H94E/M95N;
      (ee) H94E/M95D;
      (ff) H94E/M95P;
      (gg) N27dE/R27fK/Q89H/H94E/M95P;
      (hh) N27dE/R27fK/Q89Y/H94E/M95P;
      (ii) N27dE/R27fK/Y91E/H94E/M95P;
      (jj) N27dE/R27fK/Y91F/H94E/M95P;
      (kk) N27dE/R27fS/Q89H/H94E/M95P;
      (ll) N27dE/R27fS/Q89Y/H94E/M95P;
      (mm) N27dE/R27fS/Y91E/H94E/M95P;
      (nn) N27dE/R27fS/Y91F/H94E/M95P;
      (oo) N27dE/R27fS/Q89H/H94Q/M95P;
      (pp) N27dE/R27fS/Q89Y/H94Q/M95P;
      (qq) N27dE/R27fS/Y91E/H94Q/M95P;
      (rr) N27dE/R27fS/Y91F/H94Q/M95P;
      (ss) N27dE/R27fV/Q89H/H94E/M95P;
      (tt) N27dE/R27fV/Q89Y/H94E/M95P;
      (uu) N27dE/R27fV/Y91E/H94E/M95P;
      (vv) N27dE/R27fV/Y91F/H94E/M95P;
      (ww) N27dE/R29S/Q89H/H94E/M95P;
      (xx) N27dE/R29S/Q89Y/H94E/M95P;
      (yy) N27dE/K30L/Q89H/H94E/M95P;
      (zz) N27dE/K30L/Q89Y/H94E/M95P;
      (aaa) S27eD/R27fK/Y91E/H94E/M95P;
      (bbb) S27eD/R27fK/Y91F/H94E/M95P;
      (ccc) S27eD/R27fK/Y91E/H94Q/M95P;
      (ddd) S27eD/R27fK/Y91F/H94Q/M95P;
      (eee) S27eD/R27fK/Y91E/H94Q;
      (fff) S27eD/R27fK/Y91F/H94Q;
      (ggg) S27eE/R27fK/Y91E/H94E/M95P;
      (hhh) S27eE/R27fK/Y91F/H94E/M95P;
      (iii) S27eE/R27fK/Y91E/H94Q/M95P;
      (jjj) S27eE/R27fK/Y91F/H94Q/M95P;
      (kkk) R27fS/R29S/Q89H/H94E/M95P;
      (lll) R27fS/R29S/Q89Y/H94E/M95P;
      (mmm) R27fS/R29S/Y91E/H94E/M95P;
      (nnn) R27fS/R29S/Y91F/H94E/M95P;
      (ooo) R27fS/R29S/Q89H/H94Q/M95P;
      (ppp) R27fS/R29S/Q89Y/H94Q/M95P;
      (qqq) R27fS/R29S/Y91E/H94Q/M95P;
      (rrr) R27fS/R29S/Y91F/H94Q/M95P;
      (sss) N27dE/R27fS/R29S/Q89Y/H94Q/M95P; and
      (ttt) N27dE/R27fS/R29S/Y91F/H94Q/M95P.
   [8] The cytosol-penetrating antibody or antigen-binding fragment thereof of any one of [1] to [7], wherein the light chain variable region comprises a human framework region (FR).
   [8A] The cytosol-penetrating antibody or antigen-binding fragment thereof of [8], wherein the light chain variable region comprises amino acid I at position 2, and/or amino acid Q at position 3 according to Kabat numbering.
   [8B] The cytosol-penetrating antibody or antigen-binding fragment thereof of [8], wherein the light chain variable region comprises a human V kappa 1 FR1 sequence.
   [8C] The cytosol-penetrating antibody or antigen-binding fragment thereof of [8], wherein the light chain variable region comprises a human V kappa 3 FR3 sequence.
   [8D] The cytosol-penetrating antibody or antigen-binding fragment thereof of [8], wherein the light chain variable region comprises a human V kappa 1 FR1, a human V kappa 1 FR2 and a human V kappa 3 FR3 sequences.
   [8E] The cytosol-penetrating antibody or antigen-binding fragment thereof of [8], wherein the light chain variable region comprises one, two, three, or four of the FR domains (FR1, FR2, FR3, and FR4) comprised in SEQ ID NO: 123.
   [8F] The cytosol-penetrating antibody or antigen-binding fragment thereof of any one of [2] to [8E], wherein the light chain variable region further comprises one, two, three or more amino acid substitutions in the CDRL2.
   [9] The cytosol-penetrating antibody or antigen-binding fragment thereof of any one of [1] to [8F], wherein the light chain variable region further comprises one, two, three or more amino acid substitutions selected from the group consisting of (position according to Kabat numbering):
      (a) substitution of amino acid W at position 50 with A, G, I, T or V;
      (b) substitution of amino acid S at position 52 with F or I;
      (c) substitution of amino acid T at position 53 with N or Y; and
      (d) substitution of amino acid R at position 54 with K or V;
      in the CDRL2 of the amino acid sequence set forth in SEQ ID NO: 4.
   [10] The cytosol-penetrating antibody or antigen-binding fragment thereof of [9], wherein the light chain variable region comprises a combination of amino acid substitutions selected from the group consisting of (position according to Kabat numbering):
      (a) L2I/V3Q/S27eD/R27fK/W50A/Y91F/H94Q;
      (b) L2I/V3Q/S27eD/R27fK/W50G/Y91F/H94Q;
      (c) L2I/V3Q/S27eD/R27fK/W50I/Y91F/H94Q;
      (d) L2I/V3Q/S27eD/R27fK/W50T/Y91F/H94Q;
      (e) L2I/V3Q/S27eD/R27fK/W50V/Y91F/H94Q;
      (f) L2I/V3Q/S27eD/R27fK/S52F/Y91F/H94Q;
      (g) L2I/V3Q/S27eD/R27fK/S52I/Y91F/H94Q;
      (h) L2I/V3Q/S27eD/R27fK/T53N/Y91F/H94Q;
      (i) L2I/V3Q/S27eD/R27fK/T53Y/Y91F/H94Q;
      (j) L2I/V3Q/S27eD/R27fK/R54K/Y91F/H94Q;
      (k) L2I/V3Q/S27eD/R27fK/W50I/S52F/Y91F/H94Q;
      (l) L2I/V3Q/S27eD/R27fK/W50I/T53N/Y91F/H94Q; and
      (m) L2I/V3Q/S27eD/R27fK/W50I/R54V/Y91F/H94Q;
      in the amino acid sequence of SEQ ID NO: 4.
   [11-1] The cytosol-penetrating antibody or antigen-binding fragment thereof of any one of [1] to [10], comprising a light chain variable region having an amino acid sequence selected from the group consisting of SEQ ID NOs: 14-150 and 161-173.
   [11-2] The cytosol-penetrating antibody or antigen-binding fragment thereof of [11-1] comprising a light chain variable region having an amino acid sequence selected from the group consisting of SEQ ID NOs: 123-150.
   [11-3] The cytosol-penetrating antibody or antigen-binding fragment thereof of [11-1] comprising a light chain variable region having an amino acid sequence selected from the group consisting of SEQ ID NOs: 161-173.
   [11A] The cytosol-penetrating antibody or antigen-binding fragment thereof of any one of [1] to [11-3], which is in the form of Fab or scFv.
   [11B] The cytosol-penetrating antibody or antigen-binding fragment thereof of any one of [1] to [11A], further comprising a heavy chain variable region, optionally a heavy chain variable region comprising an amino acid of SEQ ID NO: 158.
   [111C] The cytosol-penetrating antibody or antigen-binding fragment thereof of any one of [1] to [11B], which is a multifunctional antibody.
   [I1D] The cytosol-penetrating antibody or antigen-binding fragment thereof of [1IC], which comprises a cell surface antigen-binding domain, a cytosolic antigen-binding domain, and a cytosol-penetrating domain; wherein the cytosol-penetrating domain comprises the light chain variable region of any one of [1] to [11A].
   [12] A nucleic acid encoding the cytosol-penetrating antibody or antigen-binding fragment thereof of any one of [1] to [I1D].
   [13] A vector comprising the nucleic acid of [12].
   [14] A host cell comprising the nucleic acid of [12] or the vector of [13].
   [15] A method of producing the cytosol-penetrating antibody or antigen-binding fragment thereof of any one of [1] to [11D], comprising the steps of a) culturing the host cell of [14] under conditions suitable for the expression of the antibody or fragment thereof and b) recovering the antibody or fragment thereof.
   [16] A composition or pharmaceutical composition for delivery of a bioactive molecule into a cytoplasm comprising the cytosol-penetrating antibody or antigen-binding fragment thereof of any one of [1] to [11D].
   [17] The composition or pharmaceutical composition of [16], wherein the bioactive molecule is at least one selected from the group consisting of peptides, proteins, toxins, antibodies, antibody fragments, RNA, DNA, small molecule drugs, nanoparticles, and liposomes; wherein the bioactive molecule is conjugated or fused with the cytosol-penetrating antibody or antigen-binding fragment.
   [18] An immunoconjugate of: the cytosol-penetrating antibody or antigen-binding fragment thereof of any one of [1] to [I1D]; and a bioactive molecule.
   [19] The immunoconjugate of [18], wherein the bioactive molecule is selected from the group consisting of peptides, proteins, toxins, antibodies, antibody fragments, RNA, DNA, small molecule drugs, nanoparticles and liposomes.
   [20] A method of delivering the cytosol-penetrating antibody or antigen-binding fragment thereof of any one of [1] to [11D] or the immunoconjugate of [18] or [19] through a cell membrane and placing it in cytoplasm.
   [21] A method of targeting, inhibiting, or activating a cytosolic antigen by using the cytosol-penetrating antibody or antigen-binding fragment thereof of any one of [1] to [I1D], wherein the antibody or fragment thereof specifically binds to the cytosolic antigen.
   [21A] A method for imaging a cytosolic antigen in a sample cell, comprising contacting the cytosol-penetrating antibody or antigen-binding fragment thereof of any one of [1] to [I1D] with the sample cell, wherein the antibody or fragment thereof is labeled and specifically binds to the cytosolic antigen.
   [22] A method of producing a cytosol-penetrating antibody or antigen-binding fragment thereof, wherein the method comprises:
      (a) providing a parent cytosol-penetrating antigen-binding molecule comprising a light chain variable region having the amino acid sequence set forth in SEQ ID NO: 4;
      (b) introducing one or more amino acid substitutions in at least one selected from CDRL1 and CDRL3 in the light chain variable region of the parent cytosol-penetrating antigen-binding molecule; and
      (c) identifying and/or selecting a cytosol-penetrating antibody or antigen-binding fragment thereof comprising the one or more amino acid substitutions which exhibits enhanced cytosol-penetrating ability as compared to the parent cytosol-penetrating antigen-binding molecule.
   [22A] The method of [22], further comprising identifying and/or selecting a cytosol-penetrating antibody or antigen-binding fragment thereof which exhibits one or more properties relative to the parent antibody, wherein the one or more properties are selected from the group consisting of:
      (a) increased antibody expression level;
      (b) reduced binding activity to extracellular matrix (ECM), preferably as determined by a method described in Reference Example 2; and
      (c) improved physicochemical property, preferably as indicated by higher monomer peak percentage as determined by size-exclusion chromatography (SEC) analysis.
   [23] The method of any one of [22] to [22A], further comprising:
      (d) obtaining an expression vector that comprises a gene encoding the cytosol-penetrating antibody or antigen-binding fragment thereof produced by the method of [22] and an operably linked suitable promoter;
      (e) introducing the vector into a host cell and culturing the host cell to allow production of the cytosol-penetrating antibody or antigen-binding fragment thereof; and
      (f) recovering the cytosol-penetrating antibody or antigen-binding fragment thereof from the host cell culture.
   [24] The method of any one of [22] to [23], wherein the one or more amino acid substitutions are located at Kabat numbering positions 24 to 34 and 89 to 97 in the amino acid sequence set forth in SEQ ID NO: 4.
   [25] The method of [24], wherein the one or more amino acid substitutions are located at Kabat numbering positions 24 to 32 and 93 to 97 in the amino acid sequence set forth in SEQ ID NO: 4.
   [26] The method of any one of [22] to [25], wherein the one or more amino acid substitutions are selected from the group consisting of (position according to Kabat numbering):
      (a) substitution of amino acid K at position 24 with A, D, E, F, G, H, I, L, N, Q, R, S, T, V or Y;
      (b) substitution of amino acid S at position 25 with A, D, E, F, I, M, N, T, V or Y;
      (c) substitution of amino acid S at position 26 with A, D, E, F, G, H, I, K, M, N, P, R, Tor V;
      (d) substitution of amino acid Q at position 27 with A, D, E, F, H, I, L, M, S, or Y;
      (e) substitution of amino acid S at position 27a with D;
      (f) substitution of amino acid L at position 27b with I, P, Q, T or V;
      (g) substitution of amino acid F at position 27c with A, D, E, I, M, N, S, W or Y;
      (h) substitution of amino acid N at position 27d with A, D, E, F, H, N or Q;
      (i) substitution of amino acid S at position 27e with A, D, E, I, L, M, N, P, Q, T, V or Y;
      (j) substitution of amino acid R at position 27f with A, E, F, G, H, I, K, L, M, N, Q, S, T, V, W or Y;
      (k) substitution of amino acid T at position 28 with E, H, I, Q, S, V, W or Y;
      (l) substitution of amino acid R at position 29 with A, D, E, F, G, H, I, K, L, M, N, P, Q, S, T, V, W or Y;
      (m) substitution of amino acid K at position 30 with A, D, E, F, H, I, L, M, N, P, Q, S, T, V, W or Y;
      (n) substitution of amino acid N at position 31 with D, M, S or T;
      (o) substitution of amino acid Y at position 32 with D, E, G, H or Q;
      (p) substitution of amino acid Q at position 89 with H, I, T or Y;
      (q) substitution of amino acid Y at position 91 with E, F or N;
      (r) substitution of amino acid Y at position 93 with I, L, M, N, P, Q, S, T, V or W;
      (s) substitution of amino acid H at position 94 with A, D, E, F, G, I, L, M, N, P, Q, R, S, T, V, W or Y;
      (t) substitution of amino acid M at position 95 with A, D, E, F, G, H, I, K, L, N, P, Q, R, S, T, V, W or Y;
      (u) substitution of amino acid Y at position 96 with A, D, E, F, G, H, K, M, N, P, Q, S, T or V; and
      (v) substitution of amino acid T at position 97 with A, D, E, G, H, K, P, Q, R, S or V;
      in the amino acid sequence set forth in SEQ ID NO: 4.
   [26A] The method of [26], wherein each of the CDRL1 and CDRL3 in the light chain variable region comprises two or more amino acid substitutions selected from the group defined in [26](a) to (v).
   [27] The method of any one of [22] to [26], wherein said one or more amino acid substitutions enhance the cytosol-penetrating activity of the antibody or fragment thereof as compared to a reference antibody or fragment thereof without said substitutions and comprising a light chain variable region having the amino acid sequence set forth in SEQ ID NO: 4.
   [28] The method of any one of [22] to [27], wherein the one or more amino acid substitutions are selected from the group consisting of (position according to Kabat numbering):
      (a) substitution of amino acid K at position 24 with N;
      (b) substitution of amino acid Q at position 27 with E;
      (c) substitution of amino acid S at position 27a with D;
      (d) substitution of amino acid N at position 27d with D or E;
      (e) substitution of amino acid S at position 27e with D or E;
      (f) substitution of amino acid R at position 27f with D, H, K, L, S or V;
      (g) substitution of amino acid T at position 28 with D;
      (h) substitution of amino acid R at position 29 with E, G, M or S;
      (i) substitution of amino acid K at position 30 with L or Q;
      (j) substitution of amino acid Y at position 32 with E;
      (k) substitution of amino acid Q at position 89 with H, I, T or Y;
      (l) substitution of amino acid Y at position 91 with E, F or N;
      (m) substitution of amino acid Y at position 93 with E;
      (n) substitution of amino acid H at position 94 with E, P or Q;
      (o) substitution of amino acid M at position 95 with D, N or P; and
      (p) substitution of amino acid Y at position 96 with E or P;
      in the amino acid sequence set forth in SEQ ID NO: 4.
   [28A] The method of [28], wherein each of the CDRL1 and CDRL3 in the light chain variable region comprises two or more amino acid substitutions selected from the group defined in [28](a) to (p).
   [29] The method of any one of [22] to [28], wherein the one or more amino acid substitutions comprise a combination of amino acid substitutions selected from the group consisting of (position according to Kabat numbering):
      (a) S27eD/R27fK;
      (b) S27eD/R27fH;
      (c) N27dE/S27eD;
      (d) S27eD/T28D;
      (e) S27eD/R29E;
      (f) S27eD/R29S;
      (g) K24N/S27eD;
      (h) N27dD/R27fK;
      (i) N27dE/R27fK;
      (j) R27fK/T28D;
      (k) R27fK/R29E;
      (1) R27fK/R29S;
      (m) K24N/R27fK;
      (n) K24N/N27dD;
      (o) N27dD/T28D;
      (p) N27dD/R29E;
      (q) N27dD/R29S;
      (r) S27eD/Q89H
      (s) N27dD/Q89H;
      (t) R27fK/Q89H;
      (u) S27eD/Y91E;
      (v) N27dD/Y91E;
      (w) R27fK/Y91E;
      (x) Q89H/H94P;
      (y) Y91E/H94P;
      (z) H94P/M95N;
      (aa) H94P/M95D;
      (bb) H94P/M95P;
      (cc) H94E/Y96P;
      (dd) H94E/M95N;
      (ee) H94E/M95D;
      (ff) H94E/M95P;
      (gg) N27dE/R27fK/Q89H/H94E/M95P;
      (hh) N27dE/R27fK/Q89Y/H94E/M95P;
      (ii) N27dE/R27fK/Y91E/H94E/M95P;
      (jj) N27dE/R27fK/Y91F/H94E/M95P;
      (kk) N27dE/R27fS/Q89H/H94E/M95P;
      (ll) N27dE/R27fS/Q89Y/H94E/M95P;
      (mm) N27dE/R27fS/Y91E/H94E/M95P;
      (nn) N27dE/R27fS/Y91F/H94E/M95P;
      (oo) N27dE/R27fS/Q89H/H94Q/M95P;
      (pp) N27dE/R27fS/Q89Y/H94Q/M95P;
      (qq) N27dE/R27fS/Y91E/H94Q/M95P;
      (rr) N27dE/R27fS/Y91F/H94Q/M95P;
      (ss) N27dE/R27fV/Q89H/H94E/M95P;
      (tt) N27dE/R27fV/Q89Y/H94E/M95P;
      (uu) N27dE/R27fV/Y91E/H94E/M95P;
      (vv) N27dE/R27fV/Y91F/H94E/M95P;
      (ww) N27dE/R29S/Q89H/H94E/M95P;
      (xx) N27dE/R29S/Q89Y/H94E/M95P;
      (yy) N27dE/K30L/Q89H/H94E/M95P;
      (zz) N27dE/K30L/Q89Y/H94E/M95P;
      (aaa) S27eD/R27fK/Y91E/H94E/M95P;
      (bbb) S27eD/R27fK/Y91F/H94E/M95P;
      (ccc) S27eD/R27fK/Y91E/H94Q/M95P;
      (ddd) S27eD/R27fK/Y91F/H94Q/M95P;
      (eee) S27eD/R27fK/Y91E/H94Q;
      (fff) S27eD/R27fK/Y91F/H94Q;
      (ggg) S27eE/R27fK/Y91E/H94E/M95P;
      (hhh) S27eE/R27fK/Y91F/H94E/M95P;
      (iii) S27eE/R27fK/Y91E/H94Q/M95P;
      (jjj) S27eE/R27fK/Y91F/H94Q/M95P;
      (kkk) R27fS/R29S/Q89H/H94E/M95P
      (lll) R27fS/R29S/Q89Y/H94E/M95P;
      (mmm) R27fS/R29S/Y91E/H94E/M95P;
      (nnn) R27fS/R29S/Y91F/H94E/M95P;
      (ooo) R27fS/R29S/Q89H/H94Q/M95P;
      (ppp) R27fS/R29S/Q89Y/H94Q/M95P;
      (qqq) R27fS/R29S/Y91E/H94Q/M95P;
      (rrr) R27fS/R29S/Y91F/H94Q/M95P;
      (sss) N27dE/R27fS/R29S/Q89Y/H94Q/M95P; and
      (ttt) N27dE/R27fS/R29S/Y91F/H94Q/M95P.
   [30] The method of any one of [22] to [29], further comprising replacing the framework region (FR) of the light chain variable region by a human framework region (FR).
   [30A] The method of [30], wherein the light chain variable region comprises amino acid I at position 2, and/or amino acid Q at position 3 according to Kabat numbering.
   [30B] The method of [30], wherein the light chain variable region comprises a human V kappa 1 FR1 sequence.
   [30C] The method of [30], wherein the light chain variable region comprises a human V kappa 3 FR3 sequence.
   [30D] The method of [30], wherein the light chain variable region comprises a human V kappa 1 FR1, a human V kappa 1 FR2 and a human V kappa 3 FR3 sequences.
   [30E] The method of [30], wherein the light chain variable region comprises one, two, three, or four of the FR domains (FR1, FR2, FR3, and FR4) comprised in SEQ ID NO: 123.
   [30F] The method of any one of [22] to [30], further comprising introducing one, two, three or more amino acid substitutions in the CDRL2 of the light chain variable region.
   [31] The method of any one of [22] to [30F], further comprising introducing one, two, three or more amino acid substitutions selected from the group consisting of (position according to Kabat numbering):
      (a) substitution of amino acid W at position 50 with A, G, I, T or V;
      (b) substitution of amino acid S at position 52 with F or I;
      (c) substitution of amino acid T at position 53 with N or Y; and
      (d) substitution of amino acid R at position 54 with K or V;
      in the CDRL2 of the amino acid sequence set forth in SEQ ID NO: 4.
   [32] The method of any one of [22] to [31], wherein the amino acid substitutions introduced into the light chain variable region comprise a combination of amino acid substitutions selected from the group consisting of (position according to Kabat numbering):
      (a) L2I/V3Q/S27eD/R27fK/W50A/Y91F/H94Q;
      (b) L2I/V3Q/S27eD/R27fK/W50G/Y91F/H94Q;
      (c) L2I/V3Q/S27eD/R27fK/W501/Y91F/H94Q;
      (d) L2I/V3Q/S27eD/R27fK/W50T/Y91F/H94Q;
      (e) L2I/V3Q/S27eD/R27fK/W50V/Y91F/H94Q;
      (f) L2I/V3Q/S27eD/R27fK/S52F/Y91F/H94Q;
      (g) L2I/V3Q/S27eD/R27fK/S52I/Y91F/H94Q;
      (h) L2I/V3Q/S27eD/R27fK/T53N/Y91F/H94Q;
      (i) L2I/V3Q/S27eD/R27fK/T53Y/Y91F/H94Q;
      (j) L2I1/V3Q/S27eD/R27fK/R54K/Y91F/H94Q;
      (k) L2I/V3Q/S27eD/R27fK/W50I/S52F/Y91F/H94Q;
      (1) L2I/V3Q/S27eD/R27fK/W50I/T53N/Y91F/H94Q; and
      (m) L2I/V3Q/S27eD/R27fK/W50I/R54V/Y91F/H94Q;
      in the amino acid sequence set forth in SEQ ID NO: 4.
   [33-1] The method of any one of [22] to [32], wherein the cytosol-penetrating antibody or antigen-binding fragment thereof comprises a light chain variable region having an amino acid sequence selected from the group consisting of SEQ ID NO: 14-150 and 161-173.
   [33-2] The method of [33-1], wherein the cytosol-penetrating antibody or antigen-binding fragment thereof comprises a light chain variable region having an amino acid sequence selected from the group consisting of SEQ ID NOs: 123-150.
   [33-3] The method of [33-1], wherein the cytosol-penetrating antibody or antigen-binding fragment thereof comprises a light chain variable region having an amino acid sequence selected from the group consisting of SEQ ID NOs: 161-173.
   [34] The method of any one of [22] to [33-3], wherein the cytosol-penetrating antibody or antigen-binding fragment thereof is in the form of Fab or scFv.
   [35] The method of any one of [22] to [33-3], wherein the cytosol-penetrating antibody or antigen-binding fragment thereof further comprising a heavy chain variable region, optionally a heavy chain variable region comprising an amino acid of SEQ ID NO: 158.

In another non-limiting embodiment, the present invention relates to multifunctional antigen-binding molecules (antibodies or antigen-binding fragment thereof) comprising a cytosol-penetrating domain, a cell surface antigen-binding domain, and/or a cytosolic antigen-binding domain in combination which can be delivered into cytosol in a target cell-specific manner. In one non-limiting embodiment, each domain is derived from an antibody. In one non-limiting embodiment, it is expected that these antigen-binding molecules bind to a cell surface antigen, and thereby undergo internalization in a target cell-specific manner and are transferred into cytosol.

Specifically, the present disclosure encompasses embodiments of bifunctional or multifunctional antigen-binding molecules described below for example:
[36] An antigen-binding molecule comprising a cell surface antigen-binding domain, a cytosolic antigen-binding domain, and a cytosol-penetrating domain, wherein the cytosol-penetrating domain comprises the light chain variable region of any one of [1] to [11-3].
[36A] The antigen-binding molecule of [36], which does not bind to an antigen expressed on the cell surface that is different from aforesaid cell surface antigen.
[36B] The antigen-binding molecule of [36], wherein the cytosolic antigen-binding domain and the cytosol-penetrating domain do not bind to an antigen expressed on the cell surface that is different from aforesaid cell surface antigen.
[36C] The antigen-binding molecule of [36] or [36B], wherein the cytosolic antigen-binding domain and the cytosol-penetrating domain do not bind to an antigen expressed on the cell surface.
[36D] The antigen-binding molecule of any one of [36] to [36C], wherein the antigen-binding molecule is a multifunctional antibody or a multifunctional antibody derivative.
[37] An antigen-binding molecule comprising a first and a second Fab regions, wherein
   (a) the first Fab region binds specifically to a cell surface antigen, and
   (b) the second Fab region comprises (i) a pair of a heavy chain variable region (VH) binding specifically to a cytosolic antigen and the light chain variable region (VL) of any one of [1] to [11-3].
[38] An antigen-binding molecule comprising a first and a second Fab regions and a single-chain unit, wherein
   (a) the first Fab region binds specifically to a cell surface antigen,
   (b) the second Fab region has cytosol-penetrating ability, and
   (c) the single-chain unit binds specifically to a cytosolic antigen, and
   wherein the second Fab region comprises the light chain variable region (VL) of any one of [1] to [11-3].
[39] An antigen-binding molecule comprising a first and a second Fab regions and a single-chain unit, wherein
   (a) the first Fab region binds specifically to a cytosolic antigen,
   (b) the second Fab region has cytosol-penetrating ability, and
   (c) the single-chain unit binds specifically to a cell surface antigen, and wherein the second Fab region comprises the light chain variable region (VL) of any one of [1] to [11-3].
[40] An antigen-binding molecule comprising a first and a second Fab regions and a single-chain unit, wherein
   (a) the first and the second Fab regions comprise (i) a pair of a heavy chain variable region (VH) binding specifically to a cell surface antigen and the light chain variable region (VL) of any one of [1] to [11-3], and
   (b) the single-chain unit binds specifically to a cytosolic antigen.
[41] An antigen-binding molecule comprising a first, a second, a third, and a fourth Fab regions and an Fc region, wherein
   (a) one Fab region selected from the first, the second, the third, and the fourth Fab regions binds specifically to a cell-surface antigen,
   (b) three Fab regions other than (a) comprise (i) a pair of a heavy chain variable region binding specifically to a cytosolic antigen and the light chain variable region of any one of [1] to [11-3],
   (c) the Fc region comprises a first Fc subunit and a second Fc subunit,
   (d) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.
[42] An antigen-binding molecule comprising the light chain variable region of any one of [1] to [11-3] and an Fc region, wherein the Fc region comprises one or more amino acid alterations for enhancing formation of a multimer of the antigen-binding molecule, and wherein the antigen-binding molecule has an elevated cytosol-penetrating ability as compared to an antigen-binding molecule comprising a parent Fc region which does not comprise the one or more amino acid alterations.
[43] An antigen-binding molecule comprising a first and a second Fab regions and an Fc region, wherein
   (a) the first Fab region binds specifically to a cell surface antigen,
   (b) the second Fab region comprises (i) a pair of a heavy chain variable region (VH) binding specifically to a cytosolic antigen and the light chain variable region (VL) of any one of [1] to [11], and
   (c) the Fc region comprises one or more amino acid alterations for enhancing formation of a multimer of the antigen-binding molecule.
[44] The antigen-binding molecule of any one of [37] to [43], wherein the Fab region comprising the light chain variable region of any one of [1] to [11-3] does not bind to an antigen expressed on the cell surface that is different from the cell surface antigen.
[45] The antigen-binding molecule of any one of [37] to [44], wherein the cell surface antigen is an antigen expressed specifically on a target cell, and wherein the antigen-binding molecule is delivered specifically into cytosol of the target cell.
[46] A pharmaceutical composition comprising the antigen-binding molecule of any one of [37] to [45] and a pharmaceutically acceptable carrier.
[47] A method of delivering the antigen-binding molecule of any one of [37] to [45] specifically into cytosol of a target cell, wherein the cell surface antigen is an antigen expressed specifically on the target cell.
[48] A method of removing, suppressing, or activating a cytosolic antigen in a target cell-specific manner by using the antigen-binding molecule of any one of [37] to [45], wherein the cell surface antigen is an antigen expressed specifically on the target cell.
[49] A pharmaceutical composition for diagnosing, preventing, or treating a disease in a subject, wherein a diseased cell expresses the cell surface antigen and the cytosolic antigen to which the antigen-binding molecule of any one of [37] to [45] specifically binds, and wherein the pharmaceutical composition comprises the antigen-binding molecule of any one of [37] to [45] and a pharmaceutically acceptable carrier.

### BRIEF DESCRIPTION OF DRAWINGS

FIG. 1 shows Size Exclusion Chromatography (SEC) chromatogram for the representative hT4VL.FRv04 variants. High monomer peak percentage and sharp symmetric peak shape (narrow peak width) are indicative of good physicochemical property. All of the mutated variants listed in Table 6 showed better monomer peak percentage and sharper peak shape as shown in FIG. 1.

FIG. 2 shows the results of imaging analysis for cytosol trafficking of bifunctional antibodies in ASGPR expressing HeLa cell line (HeLa/ASGPR/BirA cells). Selected antibodies in bifunctional molecular format with anti-ASGPR antibody (AGA0078) showed higher Alexa488 fluorescence intensity than an antibody in bifunctional molecular format with anti-KLH antibody (IC17=control). This indicates that the selected bifunctional antibody could be delivered into HeLa/ASGPR/BirA cells by ASGPR dependent manner, supporting receptor dependent cytosol trafficking observed by Split-NanoLuc assay (ASGPR-expressing cell specific cytosol trafficking mediated by ASGPR Ab).

FIG. 3 shows the results of Split Nluc assay for cytosol trafficking of bifunctional antibodies comprising hT4VL variant and anti-IL6R heavy chain in HepG2 cells which intrinsically expressing IL6R on the cell surface.

FIG. 4 is a schematic drawing showing the concept of the antigen-binding molecules of the present disclosure. In FIG. 4, an antigen-binding molecule in the form of a bifunctional antibody is shown for example to illustrate the concept of the target cell-specific delivery of the antigen-binding molecules of the present disclosure.

FIG. 5 is a schematic drawing showing molecular forms of multivalent antibodies, as examples of molecular forms for the antigen-binding molecules of the present disclosure.

FIG. 6 is a schematic drawing showing exemplary molecular forms of the antigen-binding molecules of the present disclosure. The antigen-binding molecules comprise a first and a second Fab regions.

FIG. 7 is a schematic drawing showing exemplary molecular forms of the antigen-binding molecules of the present disclosure. The antigen-binding molecules comprise a first and a second Fab regions and a single-chain unit.

FIG. 8 is a schematic drawing showing exemplary molecular forms of the antigen-binding molecules of the present disclosure. The antigen-binding molecules comprise a first and a second single-domain antibody variable regions and a single-chain unit.

FIG. 9 is a schematic drawing showing exemplary molecular forms of the antigen-binding molecules of the present disclosure. The antigen-binding molecules have the molecular form of DVD-Ig (registered trademark).

FIG. 10 is a schematic drawing showing exemplary molecular forms of the antigen-binding molecules of the present disclosure. The antigen-binding molecules comprise a first, a second, and a third Fab regions.

FIG. 11 is a schematic drawing showing exemplary molecular forms of the antigen-binding molecules of the present disclosure. The antigen-binding molecules comprise a first, a second, a third, and a fourth Fab regions.

FIG. 12 shows comparison of the PK of the optimized antibody variant 3D8H/hT4VL.FRv40563 and the parental 3D8 antibody (3D8H/hT4VL) which comprise identical wild type human IgG1 constant region.

FIG. 13 shows the PK of the bifunctional antibody comprising a cytosol-penetrating domain (FRv40563) and a cell surface antigen-binding domain (anti-ASGPR antibody, AGA0078). FRv40563n//AGA0078p showed faster clearance compared to its control antibody FRv40563n//IC17p, suggesting that the bifunctional antibody FRv40563n//AGA0078p could be delivered to liver by ASGPR dependent manner. In addition, FRv40563n//AGA0078p showed faster clearance than IC17n//AGA0078p. This could support ASGPR dependent cytosol trafficking in vivo.

### DESCRIPTION OF EMBODIMENTS

### 1. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “antigen-binding molecule” refers, in its broadest sense, to a molecule that specifically binds to an antigenic determinant. In one embodiment, the antigen-binding molecule is an antibody, antibody fragment, antigen-binding fragment of an antibody, or antibody derivative.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD) or “amount of bound analyte per unit amount of ligand”. Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

“Binding activity” refers to the strength of the sum total of noncovalent interactions between one or more binding sites of a molecule (e.g., an antibody) and its binding partner (e.g., an antigen). Herein, “binding activity” is not strictly limited to a 1:1 interaction between members of a binding pair (e.g., antibody and antigen).

For example, when a member of a binding pair is capable of both monovalent binding and multivalent binding, the binding activity is the sum of each binding strength. The binding activity of a molecule X for its partner Y can generally be represented by the dissociation constant (KD) or “amount of bound analyte per unit amount of ligand”. Specific illustrative and exemplary embodiments for measuring binding affinity are described in the following.

The term “antibody” herein is used in the broadest sense and encompasses various antibody structures, including but not limited to monoclonal antibodies, polyclonal antibodies, multifunctional antibodies, multispecific antibodies (e.g., bispecific antibodies), and antibody fragments so long as they exhibit the desired antigen-binding activity.

An “agonist” antigen-binding molecule or “agonist” antibody, as used herein, is an antibody which significantly potentiates a biological activity of the antigen it binds.

A “blocking” antigen-binding molecule or “blocking” antibody or an “antagonist” antigen-binding molecule or “antagonist” antibody, as used herein, is an antibody which significantly inhibits (either partially or completely) a biological activity of the antigen it binds.

The terms “antibody fragment” and “antigen-binding fragment” refers to a molecule other than an intact antibody that comprises a portion of an intact antibody. Examples of antibody fragments include but are not limited to Fv (VH and VL), Fab, Fab′, Fab′-SH, F(ab′)₂; diabodies; linear antibodies; single-chain antibody molecules (e.g. scFv); and multifunctional antibodies and multispecific antibodies that are formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

The term “cytotoxic agent” as used herein refers to a substance that inhibits or prevents a cellular function and/or causes cell death or destruction. Cytotoxic agents include, but are not limited to, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

Herein, amino acid alterations or substitutions within an Fc region or a constant region may be represented by the combination of the EU numbering system and amino acids. For example, S424N stands for substitution at position 424 in EU numbering from serine (Ser) to asparagine (Asn). EU424N stands for substitution at position 424 in EU numbering from an amino acid (any type) to asparagine (Asn).

The term “Fc receptor” or “FcR” refers to a receptor that binds to the Fc region of an antibody. An Fc receptor is a protein on the surface of immune cells such as natural killer cells, macrophages, neutrophils and mast cells. Fc receptors bind to the Fc (crystallizable fragment) region of antibodies attached to infected cells or invading pathogens, stimulate phagocytes or cytotoxic cells, and thereby destroy microorganisms or infected cells by antibody-mediated phagocytosis or antibody-dependent cell-mediated cytotoxicity. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

The term “Fc region-comprising antibody” herein refers to an antibody that comprises an Fc region. The C-terminal lysine (residue 447 according to the EU numbering system) or C-terminal glycine-lysine (residues 446-447) of the Fc region may be removed, for example, during purification of the antibody or by recombinant engineering of the nucleic acid encoding the antibody. Accordingly, a composition comprising an antibody having an Fc region according to present disclosure can comprise an antibody with G446-K447, with G446 and without K447, with all G446-K447 removed, or a mixture of three types of antibodies described above.

The terms “a subunit of an Fc domain” and “an Fc subunit” herein indicate one of the two polypeptides forming a dimeric Fc region, in other words, a polypeptide comprising a C-terminal constant region of an immunoglobulin heavy chain that is capable of stable self-association. For example, a subunit of an Fc domain of IgG comprises IgG CH2- and IgG CH3-constant domains.

“Modification that enhances association of a first Fc subunit and a second Fc subunit” is engineering of a peptide backbone or post-translational modifications on an Fc domain subunit that reduce or prevent association of a polypeptide with the same species of polypeptide, which polypeptide comprises the Fc domain subunit capable of forming a homodimer. The association-enhancing modifications used herein include separate modifications each one of which is made to one of the two Fc domain subunits for which association is desired (i.e., a first and a second subunits of an Fc domain), the modifications being complementary to each other such that it enhances association of the two Fc domain subunits. For example, the association-enhancing modifications may alter the structure or electric charge of one or both in the Fc domain for making the association thereof sterically or electrostatically desirable. Therefore, (hetero-)dimerization occurs between a polypeptide comprising the first Fc domain subunit and a polypeptide comprising the second Fc domain subunit, which polypeptides may be nonidentical in that further components (e.g., an antigen-binding portion) fused to each subunit are not identical. In some embodiments, the association-enhancing modifications comprise amino acid mutations, in particular amino acid substitutions, within a Fc domain. In a certain embodiment, the association-enhancing modifications comprise different amino acid mutations, in particular amino acid substitutions, in each of the two subunits of a Fc domain.

In one embodiment, the aforementioned modifications are so-called “knob into hole” modifications, including a knob modification in one of the two subunits of an Fc domain and a hole modification in the other of the two subunits of the Fc domain.

The knob-into-hole technology is described, for example, in U.S. Pat. Nos. 5,731,168; 7,695,936; Ridgway et al., Prot Eng 9, 617-621 (1996); and Carter, J Immunol Meth 248, 7-15 (2001). Generally, this method includes introducing a projection (knob) at the interface of a first polypeptide and a corresponding cavity (hole) at the interface of a second polypeptide so that the projection can pack into the cavity to enhance heterodimer formation while to prevent homodimer formation. The projection can be constructed by substituting a small side chain of an amino acid from the interface of the first polypeptide by a larger side chain (e.g., thyrosin or tryptophan). A complementary cavity of the same or smaller size with the projection can be created at the interface of the second polypeptide by substitution a large amino acid side chain by a smaller one (e.g., alanine or threonine).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (CDRL1), 50-56 (CDRL2), 89-97 (CDRL3), 31-35b (CDRH1), 50-65 (CDRH2), and 95-102 (CDRH3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding a cytosol-penetrating antigen-binding molecule” refers to one or more nucleic acid molecules encoding a cytosol-penetrating antigen-binding molecule, including nucleic acid molecules carried on a single vector or separate vectors, and nucleic acid molecules present at a single site or a plurality of sites within a host cell. In the case that TR cytosol-penetrating antigen-binding molecule is an antibody, the “isolated nucleic acid encoding a cytosol-penetrating antigen-binding molecule” refers to one or more nucleic acid molecules encoding the heavy chain and the light chain (or fragments thereof) of the antibody, including nucleic acid molecules carried on a single vector or separate vectors, and nucleic acid molecules present at a single site or a plurality of sites within a host cell.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa and lambda, based on the amino acid sequence of its constant domain.

The term “cytosol-penetrating antigen-binding molecule” or “cytosol-penetrating antibody or antigen-binding fragment thereof” refers to an antigen-binding molecule or cytosol-penetrating antibody or antigen-binding fragment thereof that can penetrate into the cytosol of a living cell. The mode for the penetration into the cytosol is not particularly limited, and the antigen-binding molecule may be uptaken via the endocytosis process and then go through the endosome escape, or may undergo other processes. Examples other than the endosome escape include the case where an antigen-binding molecule directly penetrates through the cell membrane. The same antigen-binding molecule may penetrate into cytosol by both modes, i.e. the endosome escape and the direct penetration of the cell membrane. Antigen-binding molecules capable of penetrating into cytosol via methods other than endosome escape are reported (e.g., WO 2013102659). In one embodiment, the cytosol-penetrating antigen-binding molecules are antibodies or antibody derivatives. In another embodiment, the cytosol-penetrating antigen-binding molecules comprise a cytosol-penetrating domain.

The terms “cytosol-penetrating domain” and “region having cytosol-penetrating ability/activity” are used interchangeably to refer to a domain that allows for penetration into the cytosol of a living cell. Without wishing to be bound by theory, the mode for the penetration into the cytosol is not particularly limited, and may be through the uptake via the endocytosis process and following endosome escape, or may through other processes. The mode for the escape from endosomes is not particularly limited. For example, transfer into cytosol is achieved by the cytosol-penetrating domain in an endosome (acidic pH) interacting with the endosome membrane to create a pore on the endosome membrane. Examples other than the endosome escape include the case where an antigen-binding molecule directly penetrates through the cell membrane. The same antigen-binding molecule may penetrate into cytosol by both modes, i.e. the endosome escape and the direct penetration of the cell membrane. In one embodiment, the cytosol-penetrating domain has one or both of the endosome-escaping ability and cell membrane-permeating ability. In one embodiment, the cytosol-penetrating domain does not have an ability to be uptaken by endocytosis. In another embodiment, the cytosol-penetrating domain has an ability to be uptaken by endocytosis. The cytosol-penetrating domain may be of any type as long as it has cytosol-penetrating ability, but in one preferred embodiment, the cytosol-penetrating domain is an antibody or a fragment thereof. In the case that the cytosol-penetrating domain is an antibody or a fragment thereof, the cytosol-penetrating domain includes the whole or a portion of a combination of an antibody light chain variable region (VL) and an antibody heavy chain variable region (VH); or a single-domain antibody variable region, such as a heavy-chain antibody variable region (Variable region of Heavy chain of Heavy chain antibody: VHH), an antibody heavy chain variable region (VH), an antibody light chain variable region (VL), and a variable region of an immunoglobulin new antigen receptor (IgNAR) (VNAR). Examples of an antibody or a fragment thereof having cytosol-penetrating ability include, for example, Cytotransmab comprising a light chain variable region which exhibits cytosol-penetrating ability (Nat Commun. 2017 May 10; 8:15090). Other examples of cytosol-penetrating domains are “scFv (single chain Fv)”, “single chain antibody”, “Fv”, “scFv2 (single chain Fv 2)”, “Fab” or “F(ab′)2”, etc. The cytosol-penetrating domain may be used in combination with a peptide moiety or a nucleic acid moiety having cell penetrating activity. Examples of peptide moieties having cell penetrating activity include cell-penetrating peptides (CPP), protein transduction domains (PTD), etc. (see, for example, WO 2017156630). Examples of nucleic acid moieties having cell penetrating activity include oligo-nucleic acids having a phosphorothioate backbone (see, for example, WO 2015031837).

Whether a domain of interest is a cytosol-penetrating domain or not (whether the domain of interest has cytosol-penetrating ability or not) can be checked by various methods known to those skilled in the art. For example, assessment can be made by fusing the domain of interest with a molecule having no cytosol-penetrating ability (e.g., an antigen-binding molecule), contacting the fused molecule with a cell, and then seeing if the fused molecule is detected in the cytosol of the cell. Various methods are known for detecting the presence of a molecule of interest in cytosol, and, for example, an assay using split NanoLuc (registered trademark) system and imaging analysis using fluorescence microscopy described in the Reference Examples 1 and 3 respectively below are known. BirA assay and an assay using split GFP system can be also used for detecting the presence of a molecule of interest in cytosol.

In one embodiment, the cytosol-penetrating domain may interact with a cell surface antigen and/or a cytosolic antigen.

“Cytosol-trafficking” ability of an antigen-binding molecule means the ability of the antigen-binding molecule to traffic to the cytosol, and the term “penetrate”, “traffic” or “traffick” are used interchangeably. The terms “cytosol-penetrating ability/activity “cytosol-trafficking ability/activity”, “cytoplasmic permeability”, and “permeability to the cytoplasm” of an antigen-binding molecule are used herein interchangeably to refer to the ability of the antigen-binding molecule to translocate itself from the endosomes into the cytosol, or to traffic to the cytosol.

“Reduced extracellular matrix (ECM)-binding” or “lower ECM binding” of an antigen-binding molecule (antibody) means that binding of the antigen-binding molecule to ECM is reduced as compared to a reference antibody. ECM (extracellular matrix) is an extracellular constituent and resides at various sites in vivo. Therefore, an antibody strongly binding to ECM is known to have poorer kinetics in blood (shorter half-life) (WO2012093704 A1). Thus, an antigen-binding molecule comprising amino acid substitutions that enhance cytosol-penetrating activity but do not increase ECM binding is preferably selected as the antigen-binding molecule variants that appear in the antibody library.

The binding of each antibody to ECM (extracellular matrix) can be evaluated by the following procedures with reference to WO2012093704 A1: ECM Phenol red free (BD Matrigel #356237) was diluted to 2 mg/mL with TBS and 5 micro L was added to the center of each well of an ECL assay plate (L15XB-3, MSD K.K., high bind) cooled on ice. Then, the coated plate was sealed and kept overnight at 4 degrees C. The assay was then conducted at room temperature the following day. 150 micro L of ECL blocking buffer (PBS supplemented with 0.5% BSA and 0.05% Tween 20) was added to each well of the coated plate, and the plate was left standing at room temperature for 2 hours or longer. Next, the blocking buffer was removed. Each antibody sample was diluted to 9 micro g/mL with ACES-T and further diluted to 3 micro g/mL with ECLDB (PBS supplemented with 0.1% BSA and 0.01% Tween 20). 50 micro L of the diluted antibody solution was added to each well and incubated at 30 degrees C. for 1 hour with shaking at 600 rpm. The samples were then removed by tapping the inverted plate. Next, 50 micro L of glutaraldehyde was added to each well to fix the samples and incubated at room temperature for 10 minutes. The wells were then washed thrice with PBS-T (PBS supplemented with 0.05% Tween 20). A secondary antibody was diluted to 1 micro g/mL with ECLDB and 50 micro L of the secondary antibody solution was added to each well of the plate. The plate was sealed and incubated at 30 degrees C. for 1 hour at 600 rpm while shielded from light. Next, the secondary antibody solution was removed. The plate was washed thrice again with PBS-T before adding READ buffer (MSD K.K.), which was added at 150 micro L/well, followed by immediate detection of luminescence signal using Sector Imager 2400 (MSD K.K.).

The term “lower aggregation tendency” of an antigen-biding molecule means that self-association of antigen-binding molecules with each other, i.e., interactions between monomers is reduced as compared to that of reference antigen-binding molecules. Aggregation profile was evaluated by size-exclusion chromatography (SEC) analysis using UPLC (Acquity H-Class bio, Waters) for each variant. 50 mM Phosphate/300 mM NaCl (pH 7.0) was used for running buffer and BEH200 column (Waters, #186005225) was used as analytical column. Chromatogram was recorded by 220 nM UV absorption. Data was processed using Empower3 Software (Waters), and monomer peak percentage and full width at half maximum of the monomer peak were calculated. High monomer peak percentage and sharp symmetric peak shape (narrow peak width) are considered as good physicochemical property.

“Fuse/fusing/fusion” means that components (e.g., a Fab region and an Fc domain subunit) are linked together directly or by peptide bond via one or more peptide linkers.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen-binding molecules of the present disclosure are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The phrase “substantially reduced”, “substantially increased”, or “substantially different,” as used herein, refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., KD values).

The term “substantially similar”, “substantially unchanged”, or “substantially the same,” as used herein, refers to a sufficiently high degree of similarity between two numeric values (for example, one associated with an antigen-binding molecule of the present disclosure and the other associated with a reference/comparator antigen-binding molecule), such that one of skill in the art would consider the difference between the two values to be of little or no biological and/or statistical significance within the context of the biological characteristic measured by said values (e.g., KD values).

The expression “substantially not detected” used herein means that something is on or below the detection limit in standard detection techniques known to those skilled in the art (e.g., Western blotting, capillary immunoassay, imaging by fluorescent microscopy, etc.), which means that the something is totally not detected or, even when detected the detection level is near the background or noises.

Specifically, the antigen-binding molecules of the present disclosure are said as being substantially not detected, for example, when measurements for the antigen-binding molecules of the present disclosure are substantially similar to or substantially the same as (e.g., there is no statistical significance from) the measurements for negative controls.

The expression “substantially not delivered” means that the delivery of something is substantially not detected in standard detection techniques for detecting the delivery. For example, for the delivery into the cytosol of a cell, an antigen-binding molecule is said as being substantially not delivered into the cytosol of a cell when the amount of the antigen-binding molecule of the present disclosure or a complex thereof in the cytosol of a cell having been contacted with the antigen-binding molecule or a complex thereof is substantially similar to or substantially the same as (e.g., there is no statistical significance from) the amount in the cytosol of a cell for which no such contact has been made.

Similarly, the expression substantially does not remove, suppress, or activate means that the removal, suppression, activation is substantially not detected in standard detection techniques for detecting them. For example, for the removal, suppression, activation of a cytosolic antigen, the cytosolic antigen is said as being substantially not removed, suppressed, or activated when the measurements in the case of using the antigen-binding molecule of the present disclosure or a complex thereof are substantially similar to or substantially the same as (e.g., there is no statistical significance from) the measurements for controls.

### I. Compositions and Methods

I In one aspect, the present disclosure is based, in part, on the finding of antigen-binding molecules (e.g. antibodies) comprising a light chain variable region with improved cytosol-penetrating ability or activity as compared to an already existing cytosol-penetrating antibody light chain variable region (VL of h3D8 antibody: hT4VL disclosed in WO2016/013870). The present disclosure is further based, in part, on the finding that antigen-binding molecules (antibodies) having improved cytosol-penetrating antibody further show improved expression level, reduced ECM (extracellular matrix) binding, and/or improved physicochemical property indicated by lower aggregation tendency. In certain embodiments, cytosol-penetrating antigen-binding molecules (antibodies) or fragments thereof provided herein comprise a light chain variable region comprising CDRL1, CDRL2, and CDRL3 of hT4VL.FRv4 (SEQ ID NO: 4), which only differs from hT4VL by having a human V kappa 3 (VK3) FR3 in place of a human V kappa 1 FR3, and comprising one or more amino acid substitutions in at least one selected from the CDRL1, CDRL2, and CDRL3, preferably from the CDRL1 and CDRL3. In another aspect, multifunctional antigen-binding molecules provided herein comprises the cytosol-penetrating antibody or a fragment of the antibody, an antibody binding to a cell surface antigen or a fragment of the antibody, and/or an antibody binding to a cytosolic antigen or a fragment of the antibody in combination. The multifunctional antigen-binding molecules are delivered into cytosol in a target cell-specific manner. In certain embodiments, multifunctional antigen-binding molecules provided herein comprises a cell surface antigen-binding domain, a cytosolic antigen-binding domain, and a cytosol-penetrating domain comprising the aforementioned light chain variable region. The antigen-binding molecules of the present disclosure are useful, for example, for diagnosis, prevention, or treatment of a disease that arises due to the cytosolic antigen.

A-1. Exemplary Cytosol-Penetrating Antigen-Binding Molecules (Antibodies)

In one embodiment, the antigen-binding molecules of the present disclosure are antigen-binding molecules having improved (enhanced) cytosol-penetrating ability (activity). In one embodiment, the cytosol-penetrating antigen-binding molecules of the present disclosure are more efficiently delivered (translocated) to the cytol as compared to the already exist cell-penetrating antibody. In one embodiment, a larger amount of the antigen-binding molecules is delivered to the cytosol of a target cell when a fixed amount of the cytosol-penetrating antigen-binding molecules of the present disclosure is administered to a subject or is contacted with cells, as compared to the case of the same amount of the already existing cytosol-penetrating antibodies. In one embodiment, the antigen-binding molecules of the present disclosure further show improved expression level, reduced ECM (extracellular matrix) binding, and/or improved physicochemical property indicated by lower aggregation tendency as compared to the already exist cell-penetrating antibody.

In one preferred embodiment, the cytosol-penetrating antigen-binding molecules of the present disclosure comprise a light chain variable region comprising CDRL1, CDRL2, and CDRL3 of the amino acid sequence set forth in SEQ ID NO: 4 (i.e., CDRL1, CDRL2, and CDRL3 comprised in the amino acid sequence set forth in SEQ ID NO: 4), wherein the light chain variable region comprises one or more amino acid substitutions in at least one selected from the CDRL1, CDRL2, and CDRL3. In one further preferred embodiment, the light chain variable region comprises one or more amino acid substitutions in at least one selected from the CDRL1 and CDRL3.

In one preferred embodiment, the light chain variable region comprises one or more amino acid substitutions at Kabat numbering positions 24 to 34 and 89 to 97 in the amino acid sequence set forth in SEQ ID NO: 4. More preferably, the one or more amino acid substitutions are located at Kabat numbering positions 24 to 32 and 93 to 97 in amino acid sequence set forth in SEQ ID NO: 4.

In one preferred embodiment, the light chain variable region of the antigen-binding molecules of the present disclosure comprise one or more amino acid substitutions selected from the group consisting of (position according to Kabat numbering):
(a) substitution of amino acid K at position 24 with A, D, E, F, G, H, I, L, N, Q, R, S, T, V or Y;
(b) substitution of amino acid S at position 25 with A, D, E, F, I, M, N, T, V or Y;
(c) substitution of amino acid S at position 26 with A, D, E, F, G, H, I, K, M, N, P, R, T or V;
(d) substitution of amino acid Q at position 27 with A, D, E, F, H, I, L, M, S, or Y;
(e) substitution of amino acid S at position 27a with D;
(f) substitution of amino acid L at position 27b with I, P, Q, T or V;
(g) substitution of amino acid F at position 27c with A, D, E, I, M, N, S, W or Y;
(h) substitution of amino acid N at position 27d with A, D, E, F, H, N or Q;
(i) substitution of amino acid S at position 27e with A, D, E, I, L, M, N, P, Q, T, V or Y;
(j) substitution of amino acid R at position 27f with A, E, F, G, H, I, K, L, M, N, Q, S, T, V, W or Y;
(k) substitution of amino acid T at position 28 with E, H, I, Q, S, V, W or Y;
(l) substitution of amino acid R at position 29 with A, D, E, F, G, H, I, K, L, M, N, P, Q, S, T, V, W or Y;
(m) substitution of amino acid K at position 30 with A, D, E, F, H, I, L, M, N, P, Q, S, T, V, W or Y;
(n) substitution of amino acid N at position 31 with D, M, S or T;
(o) substitution of amino acid Y at position 32 with D, E, G, H or Q;
(p) substitution of amino acid Q at position 89 with H, I, T or Y;
(q) substitution of amino acid Y at position 91 with E, F or N;
(r) substitution of amino acid Y at position 93 with I, L, M, N, P, Q, S, T, V or W;
(s) substitution of amino acid H at position 94 with A, D, E, F, G, I, L, M, N, P, Q, R, S, T, V, W or Y;
(t) substitution of amino acid M at position 95 with A, D, E, F, G, H, I, K, L, N, P, Q, R, S, T, V, W or Y;
(u) substitution of amino acid Y at position 96 with A, D, E, F, G, H, K, M, N, P, Q, S, T or V; and
(v) substitution of amino acid T at position 97 with A, D, E, G, H, K, P, Q, R, S or V;
in the amino acid sequence set forth in SEQ ID NO: 4.

In one preferred embodiment, the aforementioned one or more amino acid substitutions in the light chain variable region of the present disclosure enhance the cytosol-penetrating activity of the antibody or fragment thereof as compared to a reference antibody or fragment thereof without the substitutions and comprising a light chain variable region having the amino acid sequence set forth in SEQ ID NO: 4. In one further preferred embodiment, the antibody or fragment thereof of the present disclosure exhibits at least 1%, 5%, 8%, 10%, 12% 15%, 18%, 20%, 25%, 30%, 35%, 40%, 50%, 60%, 70%, 80%, 90%, 100%, 200% or 500% or 1000% enhanced cytosol-penetrating activity compared to a reference antibody or fragment thereof without the substitutions and comprising a light chain variable region having the amino acid sequence set forth in SEQ ID NO: 4, preferably as determined using Split Nluc assay as described in Reference Example 1. In one embodiment, the cytosol-penetrating activity is mediated by penetration of the antibody or fragment thereof into cell membrane, and/or endosomal escape.

In one preferred embodiment, the light chain variable region of the present disclosure comprises one, two, three or more amino acid substitutions selected from the group consisting of (position according to Kabat numbering):
(a) substitution of amino acid K at position 24 with N;
(b) substitution of amino acid Q at position 27 with E;
(c) substitution of amino acid S at position 27a with D;
(d) substitution of amino acid N at position 27d with D or E;
(e) substitution of amino acid S at position 27e with D or E;
(f) substitution of amino acid R at position 27f with D, H, K, L, S or V;
(g) substitution of amino acid T at position 28 with D;
(h) substitution of amino acid R at position 29 with E, G, M or S;
(i) substitution of amino acid K at position 30 with L or Q;
(j) substitution of amino acid Y at position 32 with E;
(k) substitution of amino acid Q at position 89 with H, I, T or Y;
(l) substitution of amino acid Y at position 91 with E, F or N;
(m) substitution of amino acid Y at position 93 with E;
(n) substitution of amino acid H at position 94 with E, P or Q;
(o) substitution of amino acid M at position 95 with D, N or P; and
(p) substitution of amino acid Y at position 96 with E or P;
in the amino acid sequence set forth in SEQ ID NO: 4.

In one preferred embodiment, the light chain variable region of the present disclosure comprises a combination of amino acid substitutions selected from the group consisting of (position according to Kabat numbering):
(a) S27eD/R27fK;
(b) S27eD/R27fH;
(c) N27dE/S27eD;
(d) S27eD/T28D;
(e) S27eD/R29E;
(f) S27eD/R29S;
(g) K24N/S27eD;
(h) N27dD/R27fK;
(i) N27dE/R27fK;
(j) R27fK/T28D;
(k) R27fK/R29E;
(l) R27fK/R29S;
(m) K24N/R27fK;
(n) K24N/N27dD;
(o) N27dD/T28D;
(p) N27dD/R29E;
(q) N27dD/R29S;
(r) S27eD/Q89H;
(s) N27dD/Q89H;
(t) R27fK/Q89H;
(u) S27eD/Y91E;
(v) N27dD/Y91E;
(w) R27fK/Y91E;
(x) Q89H/H94P;
(y) Y91E/H94P;
(z) H94P/M95N;
(aa) H94P/M95D;
(bb) H94P/M95P;
(cc) H94E/Y96P;
(dd) H94E/M95N;
(ee) H94E/M95D;
(ff) H94E/M95P;
(gg) N27dE/R27fK/Q89H/H94E/M95P;
(hh) N27dE/R27fK/Q89Y/H94E/M95P;
(ii) N27dE/R27fK/Y91E/H94E/M95P;
(jj) N27dE/R27fK/Y91F/H94E/M95P;
(kk) N27dE/R27fS/Q89H/H94E/M95P;
(ll) N27dE/R27fS/Q89Y/H94E/M95P;
(mm) N27dE/R27fS/Y91E/H94E/M95P;
(nn) N27dE/R27fS/Y91F/H94E/M95P;
(oo) N27dE/R27fS/Q89H/H94Q/M95P;
(pp) N27dE/R27fS/Q89Y/H94Q/M95P;
(qq) N27dE/R27fS/Y91E/H94Q/M95P;
(rr) N27dE/R27fS/Y91F/H94Q/M95P;
(ss) N27dE/R27fV/Q89H/H94E/M95P;
(tt) N27dE/R27fV/Q89Y/H94E/M95P;
(uu) N27dE/R27fV/Y91E/H94E/M95P;
(vv) N27dE/R27fV/Y91F/H94E/M95P;
(ww) N27dE/R29S/Q89H/H94E/M95P;
(xx) N27dE/R29S/Q89Y/H94E/M95P;
(yy) N27dE/K30L/Q89H/H94E/M95P;
(zz) N27dE/K30L/Q89Y/H94E/M95P;
(aaa) S27eD/R27fK/Y91E/H94E/M95P;
(bbb) S27eD/R27fK/Y91F/H94E/M95P;
(ccc) S27eD/R27fK/Y91E/H94Q/M95P;
(ddd) S27eD/R27fK/Y91F/H94Q/M95P;
(eee) S27eD/R27fK/Y91E/H94Q;
(fff) S27eD/R27fK/Y91F/H94Q;
(ggg) S27eE/R27fK/Y91E/H94E/M95P;
(hhh) S27eE/R27fK/Y91F/H94E/M95P;
(iii) S27eE/R27fK/Y91E/H94Q/M95P;
(jjj) S27eE/R27fK/Y91F/H94Q/M95P;
(kkk) R27fS/R29S/Q89H/H94E/M95P;
(lll) R27fS/R29S/Q89Y/H94E/M95P;
(mmm) R27fS/R29S/Y91E/H94E/M95P;
(nnn) R27fS/R29S/Y91F/H94E/M95P;
(ooo) R27fS/R29S/Q89H/H94Q/M95P;
(ppp) R27fS/R29S/Q89Y/H94Q/M95P;
(qqq) R27fS/R29S/Y91E/H94Q/M95P;
(rrr) R27fS/R29S/Y91F/H94Q/M95P;
(sss) N27dE/R27fS/R29S/Q89Y/H94Q/M95P; and
(ttt) N27dE/R27fS/R29S/Y91F/H94Q/M95P.

In one preferred embodiment, the light chain variable region of the present disclosure comprises a human framework region(s) (FR(s)). In certain embodiments, the light chain variable region comprises amino acid I at position 2, and/or amino acid Q at position 3 according to Kabat numbering.

In one preferred embodiment, the light chain variable region of the present disclosure may comprise a human V kappa 1, V kappa 2, or V kappa 3 FR1 sequence. More preferably, the light chain variable region comprises a human V kappa 1 FR1 sequence. In one preferred embodiment, the light chain variable region may comprise a human V kappa 1, V kappa 2, or V kappa 3 FR2 sequence. More preferably, the light chain variable region comprises a human V kappa 1 FR2 sequence. In one preferred embodiment, the light chain variable region may comprise a human V kappa 1, V kappa 2, or V kappa 3 FR3 sequence. More preferably, the light chain variable region comprises a human V kappa 1 FR3 sequence. In one further preferred embodiment, the light chain variable region comprises a human V kappa 1 FR1, a human V kappa 1 FR2 and a human V kappa 3 FR3 sequences.

In one preferred embodiment, the light chain variable region of the present disclosure comprises one, two, three, or four of the FR domains (FR1, FR2, FR3, and FR4) comprised in SEQ ID NO: 123.

In one preferred embodiment, the light chain variable region of the present disclosure further comprises one, two, three or more amino acid substitutions in the CDRL2. In one preferred embodiment, the one, two, three or more amino acid substitutions are located at Kabat numbering positions 50-56 in amino acid sequence set forth in SEQ ID NO: 4. In a specific embodiment, the one, two, three or more amino acid substitutions in the CDRL2 are selected from the group consisting of (position according to Kabat numbering):
(a) substitution of amino acid W at position 50 with A, G, I, T or V;
(b) substitution of amino acid S at position 52 with F or I;
(c) substitution of amino acid T at position 53 with N or Y; and
(d) substitution of amino acid R at position 54 with K or V;
in the amino acid sequence set forth in SEQ ID NO: 4.

In some embodiments, the light chain variable region of the present disclosure comprises a combination of amino acid substitutions selected from the group consisting of (position according to Kabat numbering):
(a) L2I/V3Q/S27eD/R27fK/W50A/Y91F/H94Q;
(b) L2I/V3Q/S27eD/R27fK/W50G/Y91F/H94Q;
(c) L2I/V3Q/S27eD/R27fK/W50I/Y91F/H94Q;
(d) L2I/V3Q/S27eD/R27fK/W50T/Y91F/H94Q;
(e) L2I/V3Q/S27eD/R27fK/W50V/Y91F/H94Q;
(f) L2I/V3Q/S27eD/R27fK/S52F/Y91F/H94Q;
(g) L2I/V3Q/S27eD/R27fK/S52I/Y91F/H94Q;
(h) L2I/V3Q/S27eD/R27fK/T53N/Y91F/H94Q;
(i) L2I/V3Q/S27eD/R27fK/T53Y/Y91F/H94Q;
(j) L2I1/V3Q/S27eD/R27fK/R54K/Y91F/H94Q;
(k) L2I/V3Q/S27eD/R27fK/W50I/S52F/Y91F/H94Q;
(1) L2I/V3Q/S27eD/R27fK/W50I/T53N/Y91F/H94Q; and
(m) L2I/V3Q/S27eD/R27fK/W50I/R54V/Y91F/H94Q;
in the amino acid sequence of SEQ ID NO: 4.

In one preferred embodiment, the cytosol-penetrating antigen-binding molecules of the present disclosure comprise a light chain variable region having an amino acid sequence selected from the group consisting of SEQ ID NO: 14-150 and 161-173. In one embodiment, the cytosol-penetrating antibody or antigen-binding fragment thereof produced comprises a light chain variable region having an amino acid sequence selected from the group consisting of SEQ ID NO: 123-150. In one embodiment, the cytosol-penetrating antibody or antigen-binding fragment thereof produced comprises a light chain variable region having an amino acid sequence selected from the group consisting of SEQ ID NO: 161-173.

In one further preferred embodiment, the cytosol-penetrating antigen-binding molecules of the present disclosure can be in the form of scFv (single chain Fv), single chain antibody, Fv, scFv2 (single chain Fv 2), Fab, or F(ab′)2. In one further preferred embodiment, the cytosol-penetrating antigen-binding molecules of the present disclosure can be in the form of Fab or scFv.

In one preferred embodiment, the cytosol-penetrating molecules of the present disclosure further comprise a heavy chain variable region. The cytosol-penetrating antibody or antigen-binding fragment thereof of the present disclosure may further comprise a single-domain antibody variable region, such as VHH.

### A-2. Exemplary Multifunctional Antigen-Binding Molecules Having Cytosol-Penetrating Ability

In some embodiments, the cytosol-penetrating antigen-binding molecules of the present disclosure are multifunctional antigen-binding molecules (antibodies). In one embodiment, the multifunctional antigen-binding molecules comprise a cell surface antigen-binding domain, a cytosolic antigen-binding domain, and a cytosol-penetrating domain comprising the aforementioned light chain variable region.

In one preferred embodiment, the multifunctional antigen-binding molecules of the present disclosure do not bind to antigens that are expressed on the surface of a cell and different from the aforementioned cell surface antigen. In one preferred embodiment, the cytosolic antigen-binding domain and/or the cytosol-penetrating domain (i) do not bind to antigens that are expressed on the surface of a cell and different from the aforementioned cell surface antigen; or (ii) do not bind to any antigens expressed on the surface of a cell.

It is known that one already existing cell-penetrating antibody Tmab4 (heavy chain: 3D8, light chain: hT4VL) is uptaken into intracellular endosomes by cell surface heparan sulfate proteoglycan (HSPG)-dependent endocytosis, goes through structural changes of the light chain under acidic pH in the endosomes and comes to interact with the endosome membrane to create pores on the membrane, and thereby achieves translocation into the cytosol (J Control Release. 2016 Aug. 10; 235:165-175). It is also reported that the antibody made to have reduced binding toward HSPG (Tmab4-03 (heavy chain: 3D8, light chain: hT4VL.03) has lost its ability to be uptaken by endocytosis, while maintaining pore-forming ability at pH5.5 (J Control Release. 2016 Aug. 10; 235:165-175). This means that Tmab4-03, in the form as it stands, cannot achieve translocation into cell in a cell-specific manner. In addition, it is expected that membrane deformation through interaction of Tmab4 with molecules on the endosome membrane is crucial for allowing pores to be formed in the endosome membrane (PNAS, 2011 October 108; 41; 16883-16888). Therefore, in one embodiment, it is desirable that there are multivalent cytosol-penetrating domains that interact with the endosome membrane, in order to improve cytosol-penetrating ability.

On the other hand, as described above, Tmab4 goes through endocytosis via HSPG which is universally expressed on epithelial cells, and therefore, it is difficult to deliver cytosolic antigen-binding antibodies in a target cell-specific manner. In other words, it is expected that delivery of cytosolic antigen-binding antibodies to a target tissue upon systemic administration will be low. In view of these, in one embodiment, the present inventors arrived at the idea of using a monovalent cytosol-penetrating domain to suppress nonspecific uptake, adding a target cell surface-binding domain to improve target specificity, and, at the same time, creating conditions where the interaction of antibodies and endosome membrane occurs in a multivalent fashion, in order to keep or improve cytosol-penetrating ability.

Unlike the above-described already existing antibodies, in one embodiment, the cytosol-penetrating antigen-binding molecules of the present disclosure comprise a cell surface antigen-binding domain, cytosolic antigen-binding domain, and a cytosol-penetrating domain. Therefore, in one embodiment, for the cytosol-penetrating antigen-binding molecules of the present disclosure it is expected that it binds to a cell surface antigen on a target cell and thereby undergoes endocytosis in a target cell-specific manner, and then it translocates itself from the endosomes into the cytosol. In one embodiment, the cytosol-penetrating antigen-binding molecules of the present disclosure are more selectively delivered into the cytosol of target cells as compared to the already existing cytosol-penetrating antibodies. In one embodiment, a larger amount of the antigen-binding molecules is delivered to the cytosol of a target cell when a fixed amount of the cytosol-penetrating antigen-binding molecules of the present disclosure is administered or is contacted with a subject, as compared to the case of the same amount of the already existing cytosol-penetrating antibodies. In one embodiment, the cytosol-penetrating antigen-binding molecules of the present disclosure are delivered specifically into the cytosol of a target cell, while substantially not delivered to cells that do not express the cell surface antigen to which the antigen-binding molecules specifically bind. In one embodiment, when the cytosol-penetrating antigen-binding molecules of the present disclosure are used as pharmaceuticals, the cytosol-penetrating antigen-binding molecules exert stronger drug efficacy and/or cause lesser side effects, as compared to the already existing cytosol-penetrating antibodies. In one embodiment, pharmaceutical compositions comprising the cytosol-penetrating antigen-binding molecule of the present disclosure can exert drug efficacy with smaller amount of administration and/or lesser number of administrations as compared to pharmaceutical compositions comprising the already existing cytosol-penetrating antibody.

In one further preferred embodiment, the multifunctional cytosol-penetrating antigen-binding molecules of the present disclosure may be of any one of molecular forms 1 to 8, described below.

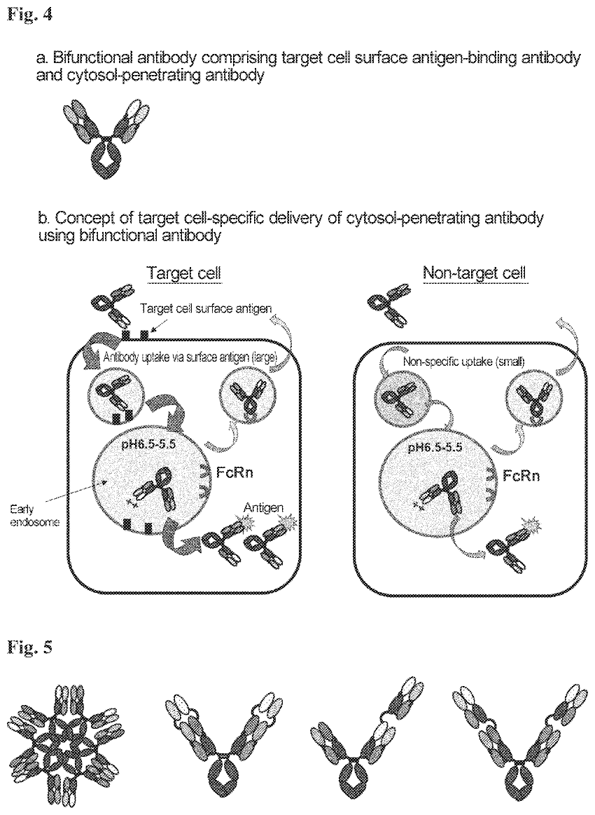

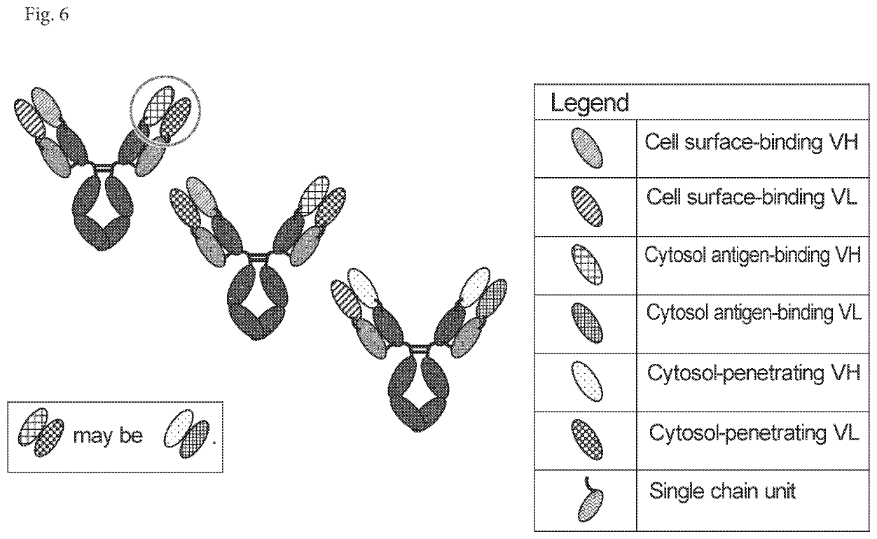

### 1. Cytosol-Penetrating Antigen-Binding Molecules Comprising a First and a Second Fab Regions (FIG. 4 and FIG. 6)

In one preferred embodiment of the present disclosure, the cytosol-penetrating antigen-binding molecules comprise a first and a second Fab regions.

In a certain embodiment, (a) the first Fab region binds specifically to a cell surface antigen; (b) the second Fab region comprises a pair of a heavy chain variable region (VH) binding specifically to a cytosolic antigen and a light chain variable region (VL) having cytosol-penetrating ability.

In another certain embodiment, (a) the first Fab region comprises a pair of a heavy chain variable region (VH) binding specifically to a cell surface antigen and a light chain variable region (VL) having cytosol-penetrating ability, (b) the second Fab region comprises a pair of a heavy chain variable region (VH) binding specifically to a cytosolic antigen and a light chain variable region (VL) having cytosol-penetrating ability.

The cytosol-penetrating antigen-binding molecules in these embodiments may further comprise an Fc region, and the Fc region may comprise a modification that enhances association of a first Fc subunit and a second Fc subunit.

The light chain variable region (VL) having cytosol-penetrating ability comprised in the cytosol-penetrating antigen-binding molecules in these embodiments is as defined in A-1. Exemplary cytosol-penetrating antigen-binding molecules (antibodies).

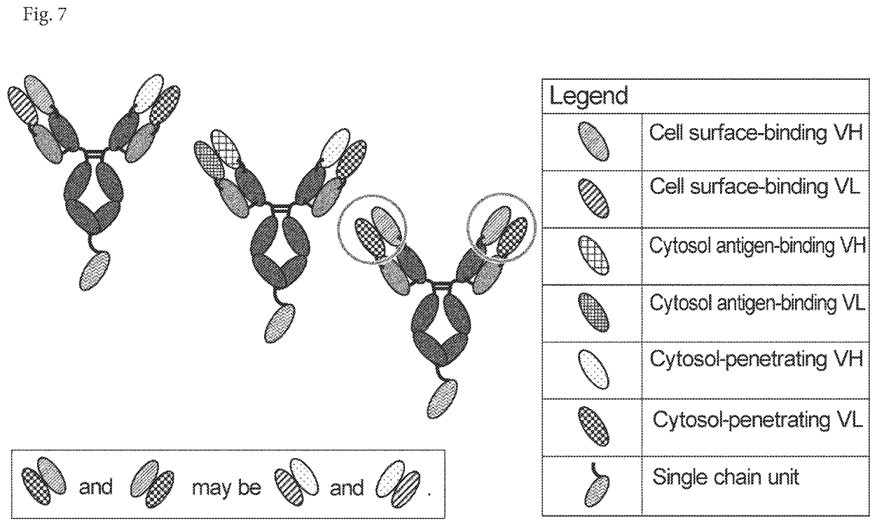

### 2. Cytosol-Penetrating Antigen-Binding Molecules Comprising a First and a Second Fab Regions and a Single-Chain Unit (FIG. 7)

In one preferred embodiment of the present disclosure, the cytosol-penetrating antigen-binding molecules comprise a first and a second Fab regions and a single-chain unit.

In a certain embodiment, (a) the first Fab region binds specifically to a cell surface antigen; (b) the second Fab region has cytosol-penetrating ability; (c) the single-chain unit binds specifically to a cytosolic antigen.

In another certain embodiment, (a) the first Fab region binds specifically to a cytosolic antigen; (b) the second Fab region has cytosol-penetrating ability; (c) the single-chain unit binds specifically to a cell surface antigen.

In yet another certain embodiment, (a) the first Fab region binds specifically to a cell surface antigen; (b) the second Fab region binds specifically to a cytosolic antigen; (c) the single-chain unit has cytosol-penetrating ability.

In yet another certain embodiment, (a) the first and the second Fab regions comprise a pair of a heavy chain variable region (VH) binding specifically to a cell surface antigen and a light chain variable region (VL) having cytosol-penetrating ability.

In a certain embodiment, the single-chain unit is fused to (i) the N terminus of the heavy chain variable region (VH) of the first Fab region and/or the N terminus of the heavy chain variable region (VH) of the second Fab region; (ii) the N terminus of the light chain variable region (VL) of the first Fab region and/or the N terminus of the light chain variable region of the second Fab region; or (iii) the C terminus of the light chain constant region (CL) of the first Fab region and/or the C terminus of the light chain constant region (CL) of the second Fab region.

The cytosol-penetrating antigen-binding molecules in these embodiments may further comprise an Fc region which comprises a first Fc subunit and a second Fc subunit, and the single-chain unit may be fused to (i) the C terminus of the first Fc subunit or (ii) the C terminus of the second Fc subunit. The first Fc subunit and the second Fc subunit may be heavy chain constant region CH2 and CH3 domains of an IgG antibody, and may comprise a modification that enhances association of the first Fc subunit and the second Fc subunit.

The single-chain unit comprised in the cytosol-penetrating antigen-binding molecules in these embodiments may be a single-domain antibody variable region or a single-chain antibody (scFv). Examples of the single-domain antibody variable region include, but are not limited to, a heavy-chain antibody variable region (VHH), a heavy chain variable region (VH), a light chain variable region (VL), and a variable region of an immunoglobulin new antigen receptor (VNAR).

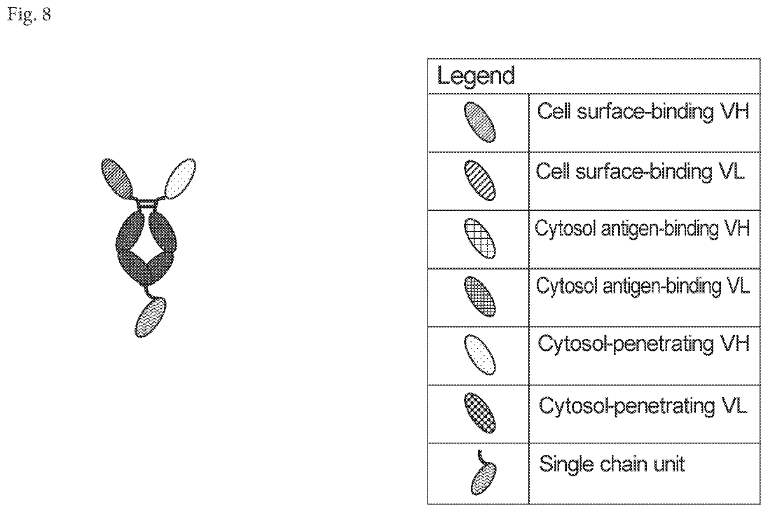

The light chain variable region (VL) having cytosol-penetrating ability comprised in the cytosol-penetrating antigen-binding molecules in these embodiments is as defined in A-1. Exemplary cytosol-penetrating antigen-binding molecules (antibodies). The Fab region having cytosol-penetrating ability and the single-chain unit having cytosol-penetrating ability comprised in the cytosol-penetrating antigen-binding molecules in these embodiments also comprises the light chain variable region having cytosol-penetrating ability. 3. Cytosol-penetrating antigen-binding molecules comprising a first and a second single-domain antibody variable regions, and a single-chain unit (FIG. 8)

In one preferred embodiment of the present disclosure, the cytosol-penetrating antigen-binding molecules comprise a first and a second single domain-antibody variable regions and a single-chain unit.

In a certain embodiment, (a) the first single-domain antibody variable region binds specifically to a cell surface antigen; (b) the second single-domain antibody variable region has cytosol-penetrating ability; (c) the single-chain unit binds specifically to a cytosolic antigen. In a certain embodiment, the single-chain unit is fused to the N terminus of the first single-domain antibody variable region and/or the N terminus of the second single-domain antibody variable region.

The cytosol-penetrating antigen-binding molecules in these embodiments may further comprise an Fc region which comprises a first Fc subunit and a second Fc subunit, and the single-chain unit may be fused to (i) the C terminus of the first Fc subunit or (ii) the C terminus of the second Fc subunit. The first Fc subunit and the second Fc subunit may be heavy chain constant region CH2 and CH3 domains of an IgG antibody, and may comprise a modification that enhances association of the first Fc subunit and the second Fc subunit.

The single-chain unit comprised in the cytosol-penetrating antigen-binding molecules in these embodiments may be a single-domain antibody variable region or a single-chain antibody (scFv). Examples of the single-domain antibody variable region include, but are not limited to, a heavy-chain antibody variable region (VHH), a heavy chain variable region (VH), a light chain variable region (VL), and a variable region of an immunoglobulin new antigen receptor (VNAR).

The single-domain antibody variable region having cytosol-penetrating ability comprised in the cytosol-penetrating antigen-binding molecules in these embodiments comprises the light chain variable region (VL) having cytosol-penetrating ability as defined in A-1. Exemplary cytosol-penetrating antigen-binding molecules (antibodies).

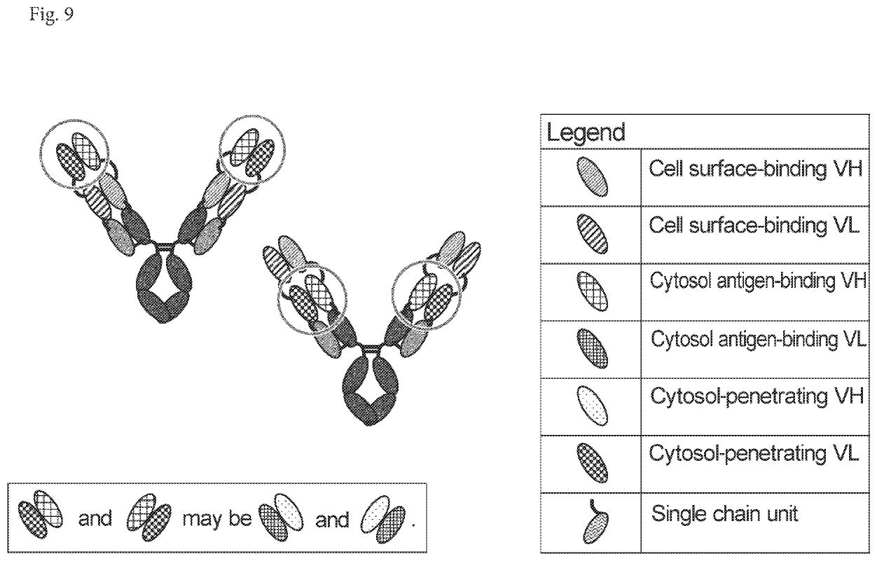

### 4. Cytosol-Penetrating Antigen-Binding Molecules Having a Molecular Form of DVD-Ig (Registered Trademark) (FIG. 9)

In one preferred embodiment, the cytosol-penetrating antigen-binding molecules of the present disclosure comprise a first and a second polypeptide chains and have the molecular form called DVD-Ig (registered trademark).

In a certain embodiment, (a) the first polypeptide chain comprises a first VD1-(X1)n-VD2-C-(X2)n, in which VD1 is a first heavy chain variable region binding specifically to a cytosolic antigen, VD2 is a second heavy chain variable region binding specifically to a cell surface antigen, C is a heavy chain constant region CH1, X1 is a linker other than CH1, X2 is an Fc region, and n is 0 or 1;
(b) the second polypeptide chain comprises a second VD1-(X1)n-VD2-C-(X2)n, in which VD1 is a first light chain variable region binding specifically to a cytosolic antigen, VD2 is a second light chain variable region binding specifically to a cell surface antigen, C is a light chain constant region CL, X1 is a linker other than CL, X2 does not comprise an Fc region, and n is 0 or 1. In another certain embodiment, (a) the first polypeptide chain comprises a first VD1-(X1)n-VD2-C-(X2)n, in which VD1 is a first heavy chain variable region having cytosol-penetrating ability, VD2 is a second heavy chain variable region binding specifically to a cell surface antigen, C is a heavy chain constant region CH1, X1 is a linker other than CH1, X2 is an Fc region, and n is 0 or 1; (b) the second polypeptide chain comprises a second VD1-(X1)n-VD2-C-(X2)n, in which VD1 is a first light chain variable region having cytosol-penetrating ability, VD2 is a second light chain variable region binding specifically to a cell surface antigen, C is a light chain constant region CL, X1 is a linker other than CL, X2 does not comprise an Fc region, and n is 0 or 1.

In yet another certain embodiment, (a) the first polypeptide chain comprises a first VD1-(X1)n-VD2-C-(X2)n, in which VD1 is a first heavy chain variable region binding specifically to a cell surface antigen, VD2 is a second heavy chain variable region binding specifically to a cytosolic antigen, C is a heavy chain constant region CH1, X1 is a linker other than CH1, X2 is an Fc region, and n is 0 or 1; (b) the second polypeptide chain comprises a second VD1-(X1)n-VD2-C-(X2)n, in which VD1 is a first light chain variable region binding specifically to a cell surface antigen, VD2 is a second light chain variable region having cytosol-penetrating ability, C is a light chain constant region CL, X1 is a linker other than CL, X2 does not comprise an Fc region, and n is 0 or 1.

The light chain variable region having cytosol-penetrating ability comprised in the cytosol-penetrating antigen-binding molecules in these embodiments is as defined in A-1. Exemplary cytosol-penetrating antigen-binding molecules (antibodies).

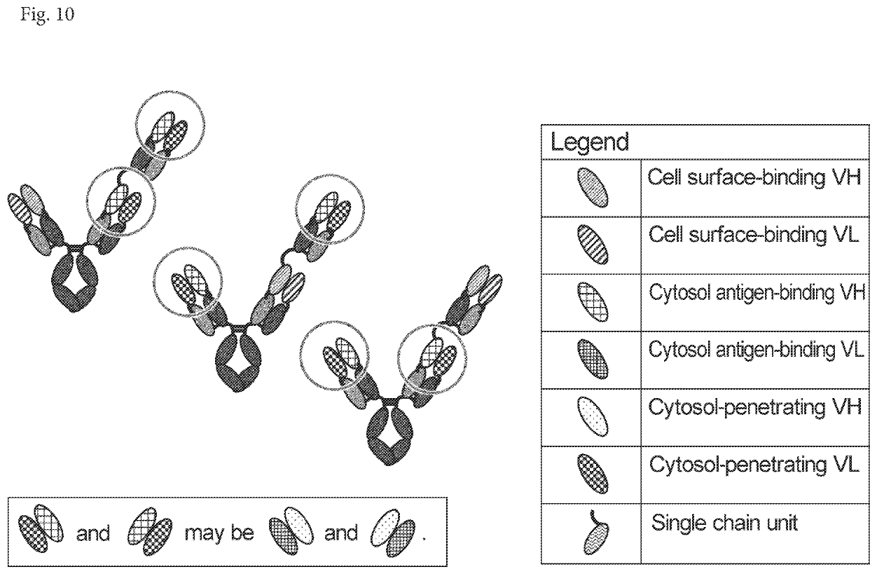

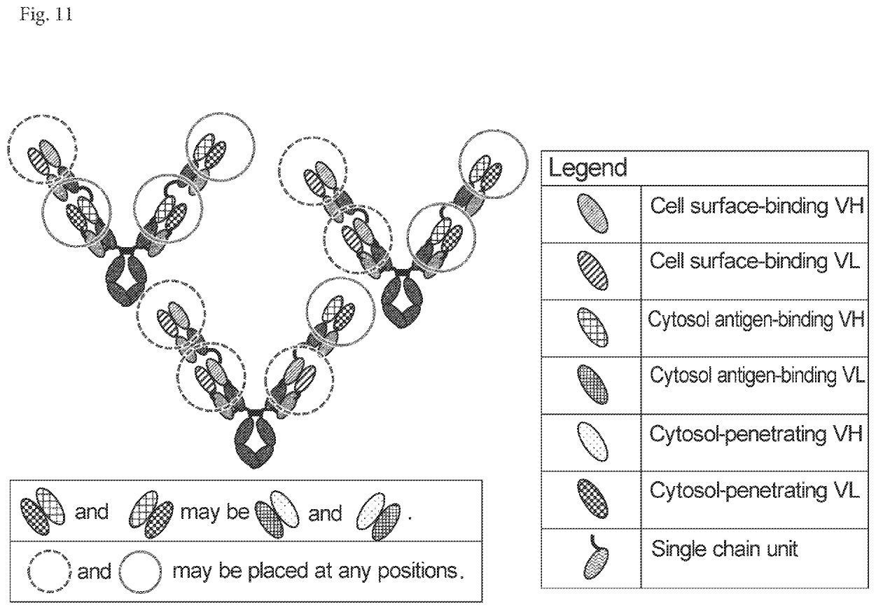

### 5. Cytosol-Penetrating Antigen-Binding Molecules Comprising a First, a Second, and a Third Fab Regions (FIG. 10)

In one preferred embodiment, the cytosol-penetrating antigen-binding molecules of the present disclosure comprise a first, a second, and a third Fab regions.

In a certain embodiment, (a) the first Fab region binds specifically to a cell surface antigen; (b) the second and the third Fab regions comprise a pair of a heavy chain variable region binding specifically to a cytosolic antigen and a light chain variable region having cytosol-penetrating ability; (c) the Fc region comprises a first Fc subunit and a second Fc subunit; (d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.

In another certain embodiment, (a) the first and the third Fab regions comprise a pair of a heavy chain variable region binding specifically to a cytosolic antigen and a light chain variable region having cytosol-penetrating ability; (b) the second Fab region binds specifically to a cell surface antigen; (c) the Fc region comprises a first Fc subunit and a second Fc subunit; (d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.

In yet another certain embodiment, (a) the first Fab region and the second Fab region comprise a pair of a heavy chain variable region binding specifically to a cytosolic antigen and a light chain variable region having cytosol-penetrating ability; (b) the third Fab region binds specifically to a cell surface antigen; (c) the Fc region comprises a first Fc subunit and a second Fc subunit; (d) the C terminus of the heavy chain of the first Fab region is fused to the N terminus of the first Fc subunit, the C terminus of the heavy chain of the second Fab region is fused to the N terminus of the second Fc subunit, and the C terminus of the heavy chain of the third Fab region is fused to the N terminus of the heavy chain of the second Fab region.

In a certain embodiment, the first Fab region and/or the second Fab region comprises any one exchange selected from (i) an exchange between a Fab light chain variable region (VL) and a Fab heavy chain variable region (VH); (ii) an exchange between a Fab light chain constant region (CL) and a Fab heavy chain constant region (CH1); and (iii) an exchange between a Fab light chain (VL-CL) and a Fab heavy chain (VH-CH1), and the first Fab region and the second Fab region do not comprise the same exchange.

In these embodiments, the first Fc subunit and the second Fc subunit may be heavy chain constant region CH2 and CH3 domains of an IgG antibody, and may comprise a modification that enhances association of the first Fc subunit and the second Fc subunit.

The light chain variable region (VL) having cytosol-penetrating ability comprised in the cytosol-penetrating antigen-binding molecules in these embodiments is as defined in A-1. Exemplary cytosol-penetrating antigen-binding molecules (antibodies).

### 6. Cytosol-Penetrating Antigen-Binding Molecules Comprising a First, a Second, a Third, and a Fourth Fab Regions (FIG. 11)

In one preferred embodiment, the cytosol-penetrating antigen-binding molecules of the present disclosure comprise a first, a second, a third, and a fourth Fab regions.

In a certain embodiment,
(a) one Fab region selected from the first, the second, the third, and the fourth Fab regions binds specifically to a cell-surface antigen; (b) three Fab regions other than (a) comprise a pair of a heavy chain variable region binding specifically to a cytosolic antigen and a light chain variable region having cytosol-penetrating ability; (d) the Fc region comprises a first Fc subunit and a second Fc subunit; (e) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.

In another certain embodiment, (a) two Fab regions selected from the first, the second, the third, and the fourth Fab regions bind specifically to a cell surface antigen; (b) two Fab regions other than (a) comprise a pair of a heavy chain variable region binding specifically to a cytosolic antigen and a light chain variable region having cytosol-penetrating ability; (d) the Fc region comprises a first Fc subunit and a second Fc subunit; (e) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.

In yet another certain embodiment, (a) three Fab regions selected from the first, the second, the third, and the fourth Fab regions bind specifically to a cell surface antigen; (b) one Fab region other than (a) comprise a pair of a heavy chain variable region binding specifically to a cytosolic antigen and a light chain variable region having cytosol-penetrating ability; (d) the Fc region comprises a first Fc subunit and a second Fc subunit; (e) the C terminus of the heavy chain of the first Fab region is fused to the N terminus of the first Fc subunit, the C terminus of the heavy chain of the second Fab region is fused to the N terminus of the second Fc subunit, the C terminus of the heavy chain of the third Fab region is fused to the N terminus of the heavy chain of the first Fab region, and the C terminus of the heavy chain of the fourth Fab region is fused to the N terminus of the heavy chain of the second Fab region.

In a certain embodiment, the first Fab region and/or the second Fab region comprise any one exchange selected from (i) an exchange between a Fab light chain variable region (VL) and a Fab heavy chain variable region (VH); (ii) an exchange between a Fab light chain constant region (CL) and a Fab heavy chain constant region (CH1); and (iii) an exchange between a Fab light chain (VL-CL) and a Fab heavy chain (VH-CH1), and the first Fab region and the second Fab region do not comprise the same exchange.

In another certain embodiment, the third Fab region and/or the fourth Fab region comprise any one exchange selected from (i) an exchange between a Fab light chain variable region (VL) and a Fab heavy chain variable region (VH); (ii) an exchange between a Fab light chain constant region (CL) and a Fab heavy chain constant region (CH1); and (iii) an exchange between a Fab light chain (VL-CL) and a Fab heavy chain (VH-CH1), and the third Fab region and the fourth Fab region do not comprise the same exchange.

In these embodiments, the first Fc subunit and the second Fc subunit may be heavy chain constant region CH2 and CH3 domains of an IgG antibody, and may comprise a modification that enhances association of the first Fc subunit and the second Fc subunit.

The light chain variable region (VL) having cytosol-penetrating ability comprised in the cytosol-penetrating antigen-binding molecules in these embodiments is as defined in A-1. Exemplary cytosol-penetrating antigen-binding molecules (antibodies). 7. Cytosol-penetrating antigen-binding molecules comprising an altered Fc region that enhances formation of a multimer (FIG. 5)

In one preferred embodiment, the cytosol-penetrating antigen-binding molecule of the present disclosure comprises a region having cytosol-penetrating ability and an Fc region, the Fc region comprising one or more amino acid alterations for enhancing formation of a multimer of the antigen-binding molecule, the antigen-binding molecule having an elevated cytosol-penetrating ability as compared to a cytosol-penetrating antigen-binding molecule comprising a parent Fc region which does not comprise the one or more amino acid alterations. The cytosol-penetrating antigen-binding molecule may further comprise a cell surface antigen-binding domain and a cytosolic antigen-binding domain.

In a certain embodiment, the cytosol-penetrating antigen-binding molecule comprises a first and a second Fab regions and an Fc region, and (a) the first Fab region binds specifically to a cell surface antigen; (b) the second Fab region comprises a pair of a heavy chain variable region (VH) binding specifically to a cytosolic antigen and a light chain variable region (VL) having cytosol-penetrating ability; (c) the Fc region comprises one or more amino acid alterations for enhancing formation of a multimer of the antigen-binding molecule. The cytosol-penetrating antigen-binding molecules comprising such an altered Fc region have an elevated cytosol-penetrating ability as compared to a cytosol-penetrating antigen-binding molecule comprising a parent Fc region which does not comprise the one or more amino acid alterations, and can allow formation of a multimeric complex of the cytosol-penetrating antigen-binding molecule that comprises a plurality of the cytosol-penetrating antigen-binding molecules.

The multimer may be a dimer, a trimer, a tetramer, a pentamer, or a hexamer. The amino acid alterations may be the combination E345R/E430G/S440Y or the combination T437R/K248E (the numbers represent positions of substitutions according to EU numbering).

In these embodiments, the first Fc subunit and the second Fc subunit may be heavy chain constant region CH2 and CH3 domains of an IgG antibody, and may comprise a modification that enhances association of the first Fc subunit and the second Fc subunit.

The light chain variable region (VL) having cytosol-penetrating ability comprised in the cytosol-penetrating antigen-binding molecules in these embodiments is as defined in A-1. Exemplary cytosol-penetrating antigen-binding molecules (antibodies).

### 8. Cytosol-Penetrating Antigen-Binding Molecules Conjugated to Heterologous Moieties

In one aspect, the antigen-binding molecule of the present disclosure is a cytosol-penetrating antigen-binding molecule, which is conjugated to a heterologous moiety. In one embodiment, the cytosol-penetrating antigen-binding molecule of the present disclosure comprises a cell surface antigen-binding domain and a cytosol-penetrating domain, and does not comprise a cytosolic antigen-binding domain. In one preferred embodiment, the cytosol-penetrating antigen-binding molecule of the present disclosure does not bind to antigens that are expressed on the surface of a cell and different from the aforementioned cell surface antigen. In one preferred embodiment, the cytosol-penetrating domain and/or the heterologous moiety (i) do not bind to antigens that are expressed on the surface of a cell and different from the aforementioned cell surface antigen; or (ii) do not bind to any antigens expressed on the surface of a cell.

In one aspect, as described above, the present inventors arrived at the idea of using a monovalent cytosol-penetrating domain to suppress nonspecific uptake, adding a target cell surface-binding domain to improve target specificity, and, at the same time, creating conditions where the interaction of antibodies and endosome membrane occurs in a multivalent fashion, in order to keep or improve cytosol-penetrating ability.

In one embodiment, the cytosol-penetrating antigen-binding molecules of the present disclosure comprise a cell surface antigen-binding domain, and a cytosol-penetrating domain. Therefore, in one embodiment, for the cytosol-penetrating antigen-binding molecules of the present disclosure it is expected that it binds to a cell surface antigen on a target cell and thereby undergoes endocytosis in a target cell-specific manner, and then it translocates itself from the endosomes into the cytosol, and thereby delivers the heterologous moieties into the cytosol.

In one embodiment, the cytosol-penetrating antigen-binding molecules of the present disclosure are more selectively delivered into the cytosol of target cells as compared to the already existing cytosol-penetrating antibodies, so that the cytosol-penetrating antigen-binding molecules of the present disclosure can deliver the heterologous moieties more selectively into the cytosol of target cells. In one embodiment, a larger amount of the heterologous moieties conjugated to the cytosol-penetrating antigen-binding molecules is delivered to the cytosol of a target cell when a fixed amount of the cytosol-penetrating antigen-binding molecules of the present disclosure is administered or is contacted with a subject, as compared to the case of the same amount of the heterologous moieties conjugated to the already existing cytosol-penetrating antibodies. In one embodiment, the heterologous moieties conjugated to the cytosol-penetrating antigen-binding molecules of the present disclosure are delivered specifically into the cytosol of a target cell, while substantially not delivered to cells that do not express the cell surface antigen to which the antigen-binding molecules specifically bind.

In one embodiment, when the cytosol-penetrating antigen-binding molecules of the present disclosure are used as pharmaceuticals, the heterologous moieties conjugated to the cytosol-penetrating antigen-binding molecules exert stronger drug efficacy and/or cause lesser side effects, as compared to the heterologous moieties conjugated to the already existing cytosol-penetrating antibodies. In one embodiment, pharmaceutical compositions comprising the heterologous moieties conjugated to the cytosol-penetrating antigen-binding molecules can exert drug efficacy with smaller amount of administration and/or lesser number of administrations as compared to pharmaceutical compositions comprising the heterologous moieties conjugated to the already existing cytosol-penetrating antibodies.

In one embodiment, the cytosol-penetrating antigen-binding molecule comprises a first and a second Fab regions, wherein (a) the first Fab region binds specifically to a cell surface antigen, and (b) the second Fab region has cytosol-penetrating ability, and wherein the antigen-binding molecule is conjugated to a heterologous moiety. In a preferred embodiment, the heterologous moiety is conjugated to the C-terminus (preferably, the biotinylated C-terminus) of the L chains of the antigen-binding molecule.

In one embodiment, the heterologous moiety conjugated to the cytosol-penetrating antigen-binding molecule is a peptide, nucleic acid, chemotherapeutic agent or drug, growth inhibitory agent, toxin (e.g., protein toxin, enzymatically active toxin of bacterial, fungal, plant, or animal origin, or a fragment thereof), or radioactive isotope. In one embodiment, the heterologous moiety is a peptide.

### A-3.

In a further aspect, the antigen-binding molecules according to any of the above-described embodiments may incorporate any characteristics described below.

The terms “antigen binding domain” and “antigen-binding fragment (of an antibody)” are used herein interchangeably to refer to a portion of an antigen-binding molecule (antibody), the portion comprising a region that specifically binds and is complementary to the whole or a portion of an antigen. The antigen binding domain may bind only to a specific portion of an antigen if the antigen is of high molecular weight. That specific portion is called “epitope”. The antigen binding domain may be provided with one or more antibody variable domains. In one preferred embodiment, the antigen binding domain comprises the whole or a portion of a combination of an antibody light chain variable region (VL) and an antibody heavy chain variable region (VH); or a single-domain antibody variable region, such as a heavy-chain antibody variable region (VHH), an antibody heavy chain variable region (VH), an antibody light chain variable region (VL), and a variable region of an immunoglobulin new antigen receptor (IgNAR) (VNAR). Other examples of antigen binding domains are “scFv (single chain Fv)”, “single chain antibody”, “Fv”, “scFv2 (single chain Fv 2)”, “Fab” or “F(ab′)2”, etc.

The terms “single-domain antibody”, “single-domain antibody variable region”, “one-domain antibody”, and “one-domain antibody variable region” used herein are not limited by the structure as long as the domain can exert antigen binding activity by itself. It is known that a typical antibody, such as IgG antibodies for example, shows antigen binding activity in a state where a variable region is formed by the pairing of VH and VL, whereas, the own domain structure of the single-domain antibody can exert antigen binding activity by itself without pairing with another domain. Usually, the single-domain antibody has a relatively low molecular weight and exists in the form of a monomer.

Examples of the single-domain antibody include, but are not limited to, antigen-binding molecules congenitally lacking a light chain, such as VHH of an animal of the family Camelidae and shark VNAR (variable region of immunoglobulin new antigen receptor: IgNAR), and antibody fragments containing the whole or a portion of an antibody VH domain or the whole or a portion of an antibody VL domain. Examples of the single-domain antibody which is an antibody fragment containing the whole or a portion of an antibody VH or VL domain include, but are not limited to, artificially prepared single-domain antibodies originating from human antibody VH or human antibody VL as described in U.S. Pat. No. 6,248,516 B1, etc. In some embodiments of the present invention, one single-domain antibody has three CDRs (CDR1, CDR2 and CDR3).

The single-domain antibody can be obtained from an animal capable of producing the single-domain antibody or by the immunization of the animal capable of producing the single-domain antibody. Examples of the animal capable of producing the single-domain antibody include, but are not limited to, animals of the family Camelidae, and transgenic animals harboring a gene capable of raising the single-domain antibody. The animals of the family Camelidae include camels, lamas, alpacas, one-hump camels and guanacos, etc. Examples of the transgenic animals harboring a gene capable of raising the single-domain antibody include, but are not limited to, transgenic animals described in International Publication No. WO2015/143414 and U.S. Patent Publication No. US2011/0123527 A1. The framework sequences of the single-domain antibody obtained from the animal may be converted to human germline sequences or sequences similar thereto to obtain a humanized single-domain antibody. The humanized single-domain antibody (e.g., humanized VHH) is also one embodiment of the single-domain antibody of the present invention. The “humanized single-domain antibody” refers to a chimeric single-domain antibody comprising amino acid residues from non-human CDRs and amino acid residues from human FRs. In an embodiment, in the humanized single-domain antibody, all or substantially all CDRs correspond to those of a non-human antibody and all or substantially all FRs correspond to those of a human antibody. Even in the case that a part of FR residues does not correspond to those from a human antibody in a humanized antibody, it is considered as one example of the case where substantially all FRs correspond to those from a human antibody. For example, to humanize a VHH, an embodiment of single-domain antibodies, a part of the FR residues has to be residues that do not correspond to those in a human antibody (C Vincke et al., The Journal of Biological Chemistry 284, 3273-3284).

The single-domain antibody can be obtained by ELISA, panning, or the like from a polypeptide library containing single-domain antibodies. Examples of the polypeptide library containing single-domain antibodies include, but are not limited to, naive antibody libraries obtained from various animals or humans (e.g., Methods in Molecular Biology 2012 911 (65-78); and Biochimica et Biophysica Acta—Proteins and Proteomics 2006 1764: 8 (1307-1319)), antibody libraries obtained by the immunization of various animals (e.g., Journal of Applied Microbiology 2014 117: 2 (528-536)), and synthetic antibody libraries prepared from antibody genes of various animals or humans (e.g., Journal of Biomolecular Screening 2016 21: 1 (35-43); Journal of Biological Chemistry 2016 291:24 (12641-12657); and AIDS 2016 30: 11 (1691-1701)).

The expression “specifically bind/binds” used herein means that one of specifically binding molecules shows binding in the condition where it does not substantially bind to molecules other than its one or more binding partner molecules. This expression is also used when the antigen binding domain has specificity for a particular epitope out of a plurality of epitopes contained in an antigen. In the case that an epitope to which the antigen-binding domain binds is contained in a plurality of different antigens, an antigen-binding molecule having that antigen-binding domain can bind to various antigens which contain the epitope.

The “cell surface antigen-binding domain” in the antigen-binding molecule of the present disclosure can bind to an epitope that is present in an antigen expressed on the surface of a cell. The “cell surface antigen” represents an antigen structure that is expressed by a cell and is present on the surface of the cell such that an antigen-binding domain can access it. In one embodiment, the cytosol-penetrating antigen-binding molecules of the present disclosure comprise a cell surface antigen-binding domain, and therefore, are specifically incorporated into cells that express the cell surface antigen (target cells). This enables specific delivery of the cytosol-penetrating antigen-binding molecules to target cells. In one embodiment, the cell surface antigen-binding domain can be provided with one or more antibody variable domains, and may be in any form as exemplified above as “antigen binding domain”.

The “cytosolic antigen-binding domain” in the antigen-binding molecules of the present disclosure can bind to an epitope that is present in an antigen expressed in the cytosol. The “cytosolic antigen” represents an antigen structure that is expressed by a cell and is present in the cytosol. Cytosolic antigen may be a protein, a nucleic acid such as DNA or RNA, or a molecule expressed in organelle such as cell nucleus and mitochondria. The cytosolic antigen-binding molecule of the present disclosure comprises a cytosolic antigen-binding domain, and therefore, can bind to the cytosolic antigen in the cytosol of a target cell and thus can neutralize, inhibit, or activate, etc. the function of the antigen. In one embodiment, the cytosolic antigen-binding domain can be provided with one or more antibody variable domains, and may be in any form as exemplified above as “antigen binding domain” In one further embodiment, the cytosolic antigen-binding domain may be enzymes or shRNAs, etc.

Herein, the phrase “the antigen-binding molecule has improved (enhanced) cytosol-penetrating ability (activity)” means that a) the amount of the antigen-binding molecule in the cytosol of cells at an arbitrary time point after allowing the antigen-binding molecule to contact with cells is increased by at least 1%, 5%, 8%, 10%, 12% 15%, 18%, 20%, 25%, 30%, 35%, 40%, 50%, 60%, 70%, 80%, 90%, 100%, 200% or 500% or 1000% or more as compared to b) the amount of a reference antigen-binding molecule present in the cytosol of cells at the same time point. In one embodiment, the reference antigen-binding molecule comprises a light chain variable region comprising CDRL1, CDRL2, and CDRL3 comprised in the amino acid sequence set forth in SEQ ID NO: 4 without any substitutions in the CDRL2, CDRL2, and CDRL3. In one embodiment, the reference antigen-binding molecule comprises a light chain variable region having the amino acid sequence set forth in SEQ ID NO: 4.

In one embodiment, when the amount of the antigen-binding molecule present in the cytosol at an arbitrary time point is expressed by HRP luminescence signal or fluorescence signal or luminescence signal, a) the HRP luminescence signal intensity or fluorescence signal intensity or luminescence signal intensity at an arbitrary time point after allowing the antigen-binding molecule to contact with cells is 0.01-fold or more, 0.05-fold or more, 0.08-fold or more, 0.1-fold or more, 0.12-fold or more, 0.15-fold or more, 0.18-fold or more, 0.2-fold or more, 0.25-fold or more, 0.3-fold or more, 0.35-fold or more, 0.4-fold or more, 0.5-fold or more, 0.6-fold or more, 0.7-fold or more, 0.8-fold or more, 0.9-fold or more, 1-fold or more, 2-fold or more, 5-fold or more, or 10-fold or more of b) the HRP luminescence signal intensity or fluorescence signal intensity or luminescence signal intensity obtained by a reference antigen-binding molecule at the same time point. In one embodiment, when the method comprising cells expressing biotin ligase (BirA) is used, the amount of the cytosol-penetrating antigen-binding molecule present in the cytosol at an arbitrary time point can be expressed by HRP luminescence signal. In a different embodiment, when the imaging analysis (such as the method described in Example 4) is used, the amount of the cytosol-penetrating antigen-binding molecule present in the cytosol at an arbitrary time point can be expressed by fluorescence signal. In a different embodiment, when the split protein system (such as the method described in Reference Example 1) is used, the amount of the cytosol-penetrating antigen-binding molecule present in the cytosol at an arbitrary time point can be expressed by luminescence signal. In one embodiment, the reference antigen-binding molecule comprises a light chain variable region comprising CDRL1, CDRL2, and CDRL3 comprised in the amino acid sequence set forth in SEQ ID NO: 4 without any substitutions in the CDRL2, CDRL2, and CDRL3. In one embodiment, the reference antigen-binding molecule comprises a light chain variable region having the amino acid sequence set forth in SEQ ID NO: 4.

In one embodiment, when it is said that a cytosol-penetrating antigen-binding molecule is delivered into the cytosol in a target cell-specific manner, it means that a) the amount of the cytosol-penetrating antigen-binding molecule present in the cytosol of non-target cells at an arbitrary time point after allowing the cytosol-penetrating antigen-binding molecule to contact with the target cells and non-target cells is small or is substantially reduced as compared to b) the amount of the cytosol-penetrating antigen-binding molecule present in the cytosol of the target cells at the same time point. In one preferred embodiment, the cytosol-penetrating antigen-binding molecule of the present disclosure is substantially not detected from the cytosol of non-target cells after allowing the cytosol-penetrating antigen-binding molecule to contact with the non-target cells. In one preferred embodiment, the cells are those derived from Hela cell line, CHO cell line, MDCK cells, or HepG2 cell line, expressing or not expressing cell surface antigens. The amount of the cytosol-penetrating antigen-binding molecule to contact with the cells may be freely selected, but the amount of the cytosol-penetrating antigen-binding molecule for the above a) and the amount of the cytosol-penetrating antigen-binding molecule for the above b) are the same. The contact between the cytosol-penetrating antigen-binding molecules and cells can be made by any methods, including incubation.

In one embodiment, the amount of the cytosol-penetrating antigen-binding molecule present in the cytosol is measured after 0 hour, 0.25 hour, 0.5 hour, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours, 13 hours, 14 hours, 15 hours, and/or 16 hours from the time of allowing the cytosol-penetrating antigen-binding molecule to contact with cells. The amount of the cytosol-penetrating antigen-binding molecule present in the cytosol may be measured only once or measured twice or more times after allowing the cytosol-penetrating antigen-binding molecule to contact with cells. By measuring the amount of the cytosol-penetrating antigen-binding molecules present in the cytosol several times, it is possible to observe over time changes in the amount of the cytosol-penetrating antigen-binding molecule present in the cytosol.

In one preferred embodiment, a) the amount of the cytosol-penetrating antigen-binding molecule present in the cytosol of non-target cells at an arbitrary time point after allowing the cytosol-penetrating antigen-binding molecule to contact with the target cells and non-target cells is 50% or less, 40% or less, 30% or less, 20% or less, 10% or less, or 5% or less of b) the amount of the cytosol-penetrating antigen-binding molecule present in the cytosol of the target cells at the same time point.

In one embodiment, when the amount of the cytosol-penetrating antigen-binding molecule present in the cytosol at an arbitrary time point is expressed by HRP luminescence signal or fluorescence signal or luminescence signal, a) the HRP luminescence signal intensity or fluorescence signal intensity or luminescence signal intensity by non-target cells at an arbitrary time point after allowing the cytosol-penetrating antigen-binding molecule to contact with the non-target cells and target cells is 0.5-fold or less, 0.4-fold or less, 0.3-fold or less, 0.2-fold or less, 0.1-fold or less, or 0.05-fold or less of b) the HRP luminescence signal intensity or fluorescence signal intensity or luminescence signal intensity by the target cells at the same time point. In one embodiment, when the method comprising cells expressing biotin ligase (BirA) is used, the amount of the cytosol-penetrating antigen-binding molecule present in the cytosol at an arbitrary time point can be expressed by HRP luminescence signal. In a different embodiment, when the imaging analysis (such as the method described in Example 4 and Reference Example 3) is used, the amount of the cytosol-penetrating antigen-binding molecule present in the cytosol at an arbitrary time point can be expressed by fluorescence signal. In a different embodiment, when the split protein system (such as the method described in Reference Example 1) is used, the amount of the cytosol-penetrating antigen-binding molecule present in the cytosol at an arbitrary time point can be expressed by luminescence signal.

In one working embodiment of the present invention, peptide linkers may be used to fuse the cell surface antigen-binding domain and the cytosol-penetrating domain, the cell surface antigen-binding domain and the cytosolic antigen-binding domain, the cytosol-penetrating domain and the cytosolic antigen-binding domain, the cell surface antigen-binding domain and an Fc subunit, the cytosol-penetrating domain and an Fc subunit, the cytosolic antigen-binding domain and an Fc subunit.

In addition, peptide linkers may be used in the antigen binding molecules having molecular forms 1 to 7 described below, in order to fuse one Fab region to another Fab region and to fuse the Fab region to an Fc subunit. For example, amino acids freely selected from Arg, Ile, Gln, Glu, Cys, Tyr, Trp, Thr, Val, His, Phe, Pro, Met, Lys, Gly, Ser, Asp, Asn, Ala, etc., particularly, Gly, Ser, Asp, Asn, Ala, in particular, Gly and Ser, and especially Gly are included.

Suitable peptide linkers can be easily selected by those skilled in the art, and suitable ones may be selected from those with different lengths, such as 1 amino acid (Gly, etc.) to 21 amino acids, 2 amino acids to 15 amino acids, or 3 amino acids to 12 amino acids (such as 4 amino acids to 10 amino acids, 5 amino acids to 9 amino acids, 6 amino acids to 8 amino acids, or 7 amino acids to 8 amino acids).

Examples of peptide linkers include, but are not limited to, glycine polymers (G)n, glycine-serine polymers (including e.g., (GS)n, (GSGGS: SEQ ID NO: 184)n and (GGGS: SEQ ID NO: 175)n, wherein n is an integer of at least 1), glycine-alanine polymers, alanine-serine polymers, and other flexible linkers well known in conventional techniques.

Among them, glycine and glycine-serine polymers are receiving attention because these amino acids are relatively unstructured and easily function as neutral tethers between components.

Examples of the flexible linker consisting of the glycine-serine polymer include, but are not limited to,

Ser
Gly-Ser (GS)
Ser-Gly (SG)
Gly-Gly-Ser (GGS)
Gly-Ser-Gly (GSG)
Ser-Gly-Gly (SGG)
Gly-Ser-Ser (GSS)
Ser-Ser-Gly (SSG)
Ser-Gly-Ser (SGS)
Gly-Gly-Gly-Ser (GGGS: SEQ ID NO: 175)
Gly-Gly-Ser-Gly (GGSG: SEQ ID NO: 176)
Gly-Ser-Gly-Gly (GSGG: SEQ ID NO: 177)
Ser-Gly-Gly-Gly (SGGG: SEQ ID NO: 178)
Gly-Ser-Ser-Gly (GSSG: SEQ ID NO: 179)
Gly-Gly-Gly-Gly-Ser (GGGGS: SEQ ID NO: 180)
Gly-Gly-Gly-Ser-Gly (GGGSG: SEQ ID NO: 181)
Gly-Gly-Ser-Gly-Gly (GGSGG: SEQ ID NO: 182)
Gly-Ser-Gly-Gly-Gly (GSGGG: SEQ ID NO: 183)
Gly-Ser-Gly-Gly-Ser (GSGGS: SEQ ID NO: 184)
Ser-Gly-Gly-Gly-Gly (SGGGG: SEQ ID NO: 185)
Gly-Ser-Ser-Gly-Gly (GSSGG: SEQ ID NO: 186)
Gly-Ser-Gly-Ser-Gly (GSGSG: SEQ ID NO: 187)
Ser-Gly-Gly-Ser-Gly (SGGSG: SEQ ID NO: 188)
Gly-Ser-Ser-Ser-Gly (GSSSG: SEQ ID NO: 189)
Gly-Gly-Gly-Gly-Gly-Ser (GGGGGS: SEQ ID
NO: 190)
Ser-Gly-Gly-Gly-Gly-Gly (SGGGGG: SEQ ID
NO: 191)
Gly-Gly-Gly-Gly-Gly-Gly-Ser (GGGGGGS: SEQ ID
NO: 192)
Ser-Gly-Gly-Gly-Gly-Gly-Gly (SGGGGGG: SEQ ID
NO: 193)
(Gly-Gly-Gly-Gly-Ser (GGGGS: SEQ ID NO: 194))n
(Ser-Gly-Gly-Gly-Gly (SGGGG: SEQ ID NO: 195)).

n [wherein n is an integer of 1 or greater] It should be noted that the length and sequence for peptide liners can be suitably selected by those skilled in the art depending on the purposes.

Amino acids contained in the amino acid sequences described herein may undergo post-translational modification (for example, modification of N-terminal glutamine into pyroglutamic acid by pyroglutamylation is well-known to those skilled in the art). Naturally, such sequences with post-translationally modified amino acids are also included in the amino acid sequences described herein.

### B. Antibodies

In a further aspect of the present disclosure, the antigen-binding molecules according to any of the above-described embodiments are antibodies and, in one preferred embodiment, monoclonal antibodies including chimeric antibodies, humanized antibodies, or human antibodies. In one embodiment, the antigen-binding molecules are antibody fragments (antigen-binding fragments) such as Fv, Fab, Fab′, scFv, diabodies, or F(ab′)₂ fragments. In another embodiment, the antibodies are full-length antibodies, for example, intact IgG1 antibodies or full-length antibodies of other antibody classes or isotypes defined herein.

In a further aspect, the antibodies according to any of the above-described embodiments may incorporate any characteristics described below in items 1 to 7, alone or in combination.

### 1. Binding Activity and Affinity of Antibodies

In certain embodiments, binding activity or affinity of antibodies provided herein is a dissociation constant (KD) of 1 micro M or less, 100 nM or less, 10 nM or less, 1 nM or less, 0.1 nM or less, 0.01 nM or less, or 0.001 nM or less (for example, 10⁻⁸ M or less, for example, 10⁻⁸ M to 10⁻¹³ M, and for example, 10⁻⁹ M to 10⁻¹³ M).

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody.

Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat′l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

### 5. Library-Derived Antibodies

Antibodies of the present disclosure may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

### 6. Multifunctional Antibodies

In certain embodiments, the antibodies provided herein are multifunctional antibodies. Multifunctional antibodies are monoclonal antibodies having functions different from one another at at least two different sites. For example, multifunctional antibodies are monoclonal antibodies having two functions, that is, (i) binding specificity and (ii) a function other than binding specificity. Examples of functions other than binding specificity include cytosol-penetrating ability. In one embodiment, the multifunctional antibodies are bifunctional antibodies (having two functions which are antigen-binding specificity and cytosol-penetrating ability). In one embodiment, the multifunctional antibodies are multispecific antibodies (e.g., bispecific antibodies). Multispecific antibodies are monoclonal antibodies having binding specificities at at least two different sites. Bispecific antibodies may be prepared as full-length antibodies or antibody fragments. In one embodiment, the multifunctional antibodies have binding specificity for an antigen and cytosol-penetrating ability. In one embodiment, the multifunctional antibodies have binding specificities for at least two different antigens and cytosol-penetrating ability.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

It will be understood that, for making multifunctional antibodies, techniques similar to the above-described techniques for making multispecific antibodies can be used.

Engineered antibodies with three or more functional antigen binding sites, including “Octopus antibodies,” are also included herein (see, e.g. US 2006/0025576A1).

The antibody or fragment herein also includes a “Dual Acting Fab” or “DAF” (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody, such as cytosol-penetrating ability. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be analyzed to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion of an enzyme (e.g. for ADEPT) or a polypeptide which increases the plasma half-life of the antibody to the N- or C-terminus of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the present disclosure may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat′l Acad. Sci. USA 83:7059-7063 (1986)) and Hellstrom, I et al., Proc. Nat′l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACT1™ non-radioactive cytotoxicity assay for flow cytometry (CellTechnology, Inc. Mountain View, CA; and CytoTox 96 (registered trademark) non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat′l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int′l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### (d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### (e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### C. Recombinant Methods and Compositions

Antibodies may be produced using recombinant methods and compositions, e.g., as described in U.S. Pat. No. 4,816,567. In one embodiment, isolated nucleic acid encoding an antigen-binding molecule described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making an antigen-binding molecule of the present disclosure is provided, wherein the method comprises culturing a host cell comprising a nucleic acid encoding the antigen-binding molecule, as provided above, under conditions suitable for expression of the antigen-binding molecule, and optionally recovering the antigen-binding molecule from the host cell (or host cell culture medium).

For recombinant production of an antigen-binding molecule of the present disclosure, nucleic acid encoding an antigen-binding molecule, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

When the antigen-binding molecule of the present disclosure is an antibody, suitable host cells for cloning or expression of vectors that encode the antibody include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### D. Assays

Antigen-binding molecules provided herein may be identified, screened for, or characterized for their physical/chemical properties and/or biological activities by various assays known in the art.

### 1. Measurement of Binding Activity and Affinity

In one embodiment, for measuring binding activity or affinity of an antibody, ligand-capturing methods using BIACORE (registered trademark) T200 or BIACORE (registered trademark) 4000 (GE Healthcare, Uppsala, Sweden), the measurement by which relies upon the principle of surface plasmon resonance analysis, are used. BIACORE (registered trademark) Control Software is used for operation of devices. In one embodiment, amine-coupling kit (GE Healthcare, Uppsala, Sweden) is used according to the manufacturer's instructions to immobilize a molecule for ligand capturing, for example, an anti-tag antibody, an anti-IgG antibody, protein A, etc., onto a sensor chip (GE Healthcare, Uppsala, Sweden) coated with carboxymethyldextran. The ligand-capturing molecule is diluted with a 10 mM sodium acetate solution at an appropriate pH and is injected at an appropriate flow rate and for an appropriate injection time. Binding activity is measured using a 0.05% polysorbate 20 (otherwise known as Tween (registered trademark)-20)-containing buffer as a measurement buffer, at a flow rate of 10-30 micro L/minute, and at a measurement temperature of preferably at 25 degrees C. or 37 degrees C. For the measurement carried out by allowing the ligand-capturing molecule to capture an antibody as a ligand, an antibody is injected to let a target amount of the antibody captured, and then a serial dilution of an antigen and/or an Fc receptor (analyte) prepared using the measurement buffer is injected. For the measurement carried out by allowing the ligand-capturing molecule to capture an antigen and/or an Fc receptor as a ligand, an antigen and/or an Fc receptor is injected to let a target amount thereof captured, and then a serial dilution of an antibody (analyte) prepared using the measurement buffer is injected.

In one embodiment, the measurement results are analyzed using BIACORE (registered trademark) Evaluation Software. For example, kinetics parameter analysis is carried out by fitting sensorgrams of association and dissociation at the same time using a 1:1 binding model, and an association rate (kon or ka), a dissociation rate (koff or kd), and an equilibrium dissociation constant (KD) may be calculated. For the case of weak binding activity, in particular, for the cases where dissociation is fast and kinetics parameters are difficult to calculate, the Steady state model may be used to calculate the equilibrium dissociation constant (KD). As additional parameters concerning binding activity, “amount of bound analyte per unit ligand amount” may be calculated by dividing the amount of bound analyte (RU) at a specific concentration by the amount of captured ligand.

### 2. Measurement of Antigen-Binding Molecules in the Cytosol

In one embodiment, an antigen-binding molecule in the cytosol is measured at an arbitrary time point after letting the antigen-binding molecule contact with a cell. The contact between the antigen-binding molecule and cell is achieved by any methods including incubation. When the antigen-binding molecule is contacted with a cell by incubation, the duration of the incubation and the period of time after the incubation and before the measurement of the antigen-binding molecule in the cytosol are arbitrarily selected. For example, in the case that an antigen-binding molecule present within the cytosol is measured only once after contacting the antigen-binding molecule with a cell, the antigen-binding molecule and the cell are incubated for 1 hour, 2 hours, 3 hours, 4 hours, 5 hours, or 6 hours, and after that the culture medium containing the antigen-binding molecule is removed and the antigen-binding molecule in the cytosol may be measured immediately. For example, in the case that the antigen-binding molecule present within the cytosol is measured for two or more times after contacting the antigen-binding molecule with a cell, the antigen-binding molecule and the cell are incubated for 1 hour, 2 hours, 3 hours, 4 hours, 5 hours, or 6 hours, and after that the culture medium containing the antigen-binding molecule is removed, and further incubation is carried out using a new culture medium free of the antigen-binding molecule for 0 hour, 0.25 hour, 0.5 hour, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours, 13 hours, 14 hours, 15 hours, and/or 16 hours, and then, the antigen-binding molecule in the cytosol may be measured. By detecting an antigen-binding molecule in the cytosol at an arbitrary time point after letting the antigen-binding molecule contact with a cell, the cytosol-penetrating ability of the antigen-binding molecule can also be detected or assessed. Therefore it is understood that the present disclosure also provides a method for detecting the cytosol-penetrating ability of the antigen binding molecule.

### a) BirA Assay

In one embodiment, the amount of an antigen-binding molecule present in the cytosol at an arbitrary time point can be evaluated in the BirA assay by detecting a fusion of a biotinylated Avi tag and the antigen-binding molecule by using a labeled biotin-binding protein. The biotin-labeled Avi tag is measured using an avidin-peroxidase conjugate and an appropriate substrate. For example, when the biotinylated Avi tag is detected using streptavidin-HRP, the amount of the antigen-binding molecule present in the cytosol can be represented by the HRP luminescence signal intensity. The BirA assay is a widely-known method among those skilled in the art and is described, for example, in W. P. R. Verdurmen et al., Journal of Controlled Release 200 (2015) 13-22 and in the Examples of the present disclosure. The conditions used for the assays to determine the amount of the antigen-binding molecule present in the cytosol may be suitably selected by those skilled in the art, and thus are not particularly limited. The fusion of the biotinylated Avi tag and the antigen-binding molecule in the cytosol may be further labeled with another labeling substance that can be detected or measured. Specifically, radioactive labels and fluorescent labels are known. The detection of labeled biotin-binding protein in the BirA assay (e.g., detection of HRP luminescence signal for the case of streptavidin-HRP) can be suitably carried out by methods known among those skilled in the art. Specifically, western blotting or capillary immuno assay (ProteinSimple, Inc.), etc. are known.

### b) Imaging by Fluorescent Microscopy

In another embodiment, the amount of the antigen-binding molecule present in the cytosol at an arbitrary time point can be evaluated by detecting using a labeled protein that binds to the antigen-binding molecule. For example, when the antigen-biding molecule comprise an IgG antibody, the antigen-binding molecule present in the cytosol can be detected using a labeled anti-IgG antibody. Regarding the label, radioactive labels and fluorescent labels are known, for example. The detection of the labeled antigen-binding molecule can be suitably carried out by methods known among those skilled in the art. For example, the labeled antigen-binding molecule can be detected by imaging the fluorescent signal using a fluorescent microscope, as described herein and in the Examples of the present disclosure.

### c) Split-GFP

In another embodiment, the amount of the antigen-binding molecule present in the cytosol at an arbitrary time point can be evaluated by detecting GFP fluorescent signal in the split-GFP complementation system. Specific methods are known to those skilled in the art and are described, for example, in WO 2016/013870. Specifically, when green fluorescent protein (GFP) is split into fragments 1-10 and fragment 11, GFP's feature of exhibiting fluorescence is removed, but the fluorescence-exhibiting feature can be recovered if the two fragments are brought close and bind to each other (Cabantous et al., 2005). To make use of this, the fragment of GFP1-10 is expressed in the cytosol and the fragment of GFP11 is fused to an arbitrary site on an antigen-binding molecule. If GFP fluorescence is observed in this method, it is indicated that the two GFP fragments bind together, i.e., the antigen-binding molecule is present in the cytosol.

### d) Improved Split Protein System

In another embodiment, the amount of the antigen-binding molecule present in the cytosol at an arbitrary time point can be evaluated by detecting a luminescent and/or fluorescent signal in the split protein system. In one embodiment, the split protein system comprises (i) a peptide comprising a mitochondrial outer membrane protein and a first fragment of a luminescent protein, and (ii) a second fragment of the luminescent protein, wherein the first fragment and the second fragment of the luminescent protein together contain the full complement of the luminescent protein. In the split protein system, the first fragment and the second fragment of the luminescent protein does not have the feature of exhibiting luminescence, but the luminescence-exhibiting feature is recovered when the first fragment of the luminescent protein comprised in the peptide binds to the second fragment of the luminescent protein. Methods to evaluate cytosol penetrating ability using Nanoluc complementation (Schaub et al., Cancer Res. (2015) 75(23):5023, Dixon et al., ACS Chem Biol. (2016) 11(2):400) were reported, but the background luminescence and/or fluorescent signal of the methods were still high. In view of these, in one embodiment, the present inventors arrived at the idea of using a mitochondrial outer membrane protein in the split protein system in order to reduce the background signal.

In one embodiment, the split luminescent protein is a split NanoLuc (registered trademark). NanoLuc is a small 19 kDa luciferase enzyme engineered from a deep sea luminous shrimp. The split NanoLuc assay utilizes NanoLuc which is cleaved into two sub-units—a small 1.3 kDa Small BiT (SmBiT) (SEQ ID NO: 202) and a larger 18 kDa Large BiT (LgBiT) (SEQ ID NO: 203). Complementation of the LgBiT and SmBiT is observed as luminescence from, enzymatic reaction on substrate, in live cells by use of the NanoLuc Live Cell Assay System (Promega). In the split NanoLuc assay, HeLa cells were genetically modified to stably express NanoLuc LgBiT intracellularly. Usually, the NanoLuc LgBiT can be expressed as free cytosolic protein. However, in one embodiment, when the NanoLuc LgBiT is thered to an intracellular structure within the cytosol, for instance, the mitochondrial outer membrane. The present inventors surprisingly discovered that the tethering of the NanoLuc LgBiT to the mitochondrial outer membrane resulted in significantly lower background signal and better assay sensitivity compared to when NanoLuc LgBiT was expressed as free cytosolic protein. On the other hand, the SmBiT conjugate can be fused to an arbitrary site on an antigen-binding molecule. Preferably, the antigen-binding molecule is an antibody and the second fragment of a luminescent protein is fused to the C-terminus of the heavy chains of the antibody. The antigen-binding molecule-SmBiT conjugates penetrate into the cytosol and thereby complement the NanoLuc LgBiT to form a luciferase enzyme, which can be detected by enhancement of luminescence.

In one example, the present disclosure provides an improved split protein system, which comprises (i) a peptide comprising a mitochondrial outer membrane protein and a first fragment of a luminescent protein, and (ii) a second fragment of the luminescent protein, wherein the first fragment and the second fragment of the luminescent protein together contain the full complement of the luminescent protein. In another example, the present disclosure provides a peptide used in the improved split protein system, wherein the peptide comprising a mitochondrial outer membrane protein and a first fragment of a luminescent protein, and wherein the first fragment and a second fragment of the luminescent protein together contain the full complement of the luminescent protein.

An example of the first fragment of the luminescent protein is NanoLuc Large BiT (LgBiT). An example of the second fragment of the luminescent protein is NanoLuc SmBiT (SmBiT). In a further embodiment, the peptide comprising the mitochondrial outer membrane protein and the first fragment of the luminescent protein further comprises a green fluorescent protein (GFP) or its variant. Preferably, the peptide comprising the mitochondrial outer membrane protein, the first fragment of the luminescent protein, and a GFP variant comprises an amino acid sequence of SEQ ID NO: 196.

A mitochondrial outer membrane protein used in the improved split protein system can be any protein which is localized on the mitochondrial outer membrane. The mitochondrial outer membrane protein can be, for example, AKAP1, BAK1, FIS1, CYB5B, FISS, FUNDC1, GDAP1, MARC1, MARC2, MFN1, MFN2, MFN3, MIEF1, MIEF2, MID49, MID51, MIGA1, MIGA2, MIRO1, MIRO2, MSTO1, MTX1, MTX2, MTX3, PGAM5, PLD6, RAB32, RMDN3, SYNJ2BP, TOM5, TOM6, TOM7, TOM20, TOM22, TOM34, TOM70, TSPO, and/or UBP30.

In a preferred embodiment, the mitochondrial outer membrane protein is an additional mitochondrial kinase anchoring protein 1 (AKAP1; also known as A-kinase anchoring protein 1). In a preferred embodiment, the mitochondrial outer membrane protein comprises an amino acid sequence of SEQ ID NO: 197. In a preferred embodiment, the peptide comprising the first fragment of the luminescent protein comprises an amino acid sequence of SEQ ID NO: 196 (AKAP1-eGFP-NanoLuc LgBit).

In one embodiment, the present disclosure provides a nucleic acid encoding the peptide comprising the mitochondrial outer membrane protein and the first fragment of the fluorescent protein, a vector comprising said nucleic acid, a cell comprising said nucleic acid or said vector, or a kit comprising said protein, nucleic acid, vector, or cell. In further embodiment, the present disclosure provides a method for detecting a target molecule present in the cytosol, which comprises (i) providing a cell comprising a nucleic acid encoding a peptide comprising a mitochondrial outer membrane protein and a first fragment of a fluorescent protein, (ii) providing a target molecule conjugated with a second fragment of the fluorescent protein, (iii) contacting the cell of (i) and the target molecule of (ii), and (iv) detecting luminescence signal in the cell of (iii), wherein the first fragment and the second fragment of the fluorescent protein together contain the full complement of the fluorescent protein. The method provided herein has lower background luminescence signal compared with a method which is the same except that the peptide of (i) does not comprise the mitochondrial outer membrane protein. In a preferred embodiment, the target molecule is a cytosol-penetrating antigen binding molecule.

In another embodiment, in the improved split protein system, any protein which is localized on any intracellular structures within the cytosol can be used instead of the mitochondrial outer membrane protein. An example of the protein localized on the intracellular structure is a nucleus outer membrane protein, and the nucleus outer membrane protein can be any protein which is localized on the nucleus outer membrane. The nucleus outer membrane protein can be, for example, NUP37, NUP43, NUP96, NUP85, NUP88, NUP107, NUP133, NUP160, NUP214, and/or NUP358.

### E. Methods of Producing an Antigen-Binding Molecule, Methods of Screening for an Antigen-Binding Molecule, and Methods of Imaging a Cytosolic Antigen

### 1. Methods of Producing an Antigen-Binding Molecule

In one aspect, the present disclosure provides methods of producing the antigen-binding molecules of the present disclosure.

In one embodiment, the method of producing comprises:
(a) obtaining an expression vector that comprises a gene encoding the antigen-binding molecule and an operably linked suitable promoter,
(b) introducing the vector into a host cell and culturing the host cell to allow production of the antigen-binding molecule, and
(c) recovering the antigen-binding molecule from the host cell culture.

In one embodiment, the method further comprises:
(d) obtaining an expression vector that comprises a gene encoding the cytosol-penetrating antibody or antigen-binding fragment thereof produced and an operably linked suitable promoter;
(e) introducing the vector into a host cell and culturing the host cell to allow production of the cytosol-penetrating antibody or antigen-binding fragment thereof; and
(f) recovering the cytosol-penetrating antibody or antigen-binding fragment thereof from the host cell culture.

In another embodiment, the method of producing comprises:
(a) providing a parent cytosol-penetrating antigen-binding molecule;
(b) introducing one or more amino acid substitutions in at least one selected from CDRL1 and CDRL3 in the light chain variable region of the parent cytosol-penetrating antigen-binding molecule; and
(c) identifying and/or selecting a cytosol-penetrating antibody or antigen-binding fragment thereof comprising the one or more amino acid substitutions which exhibit enhanced cytosol-penetrating ability as compared to the parent cytosol-penetrating antigen-binding molecule. In one preferred embodiment, the parent cytosol-penetrating antigen-binding molecule comprises a light chain variable region comprising CDRL1, CDRL2, and CDRL3 having the amino acid sequence set forth in SEQ ID NO: 4. In another preferred embodiment, the parent cytosol-penetrating antigen-binding molecule comprises a light chain variable region having the amino acid sequence set forth in SEQ ID NO: 4.

In one embodiment, the one or more amino acid substitutions introduced into the light chain variable region of the parent cytosol-penetrating antigen-binding molecule are located at Kabat numbering positions 24 to 34 and 89 to 97 in the amino acid sequence set forth in SEQ ID NO: 4. In one preferred embodiment, the one or more amino acid substitutions are located at Kabat numbering positions 24 to 32 and 93 to 97 in the amino acid sequence set forth in SEQ ID NO: 4.

In one embodiment, the one or more amino acid substitutions introduced into the light chain variable region of the parent cytosol-penetrating antigen-binding molecule are selected from the group consisting of (position according to Kabat numbering):
(a) substitution of amino acid K at position 24 with A, D, E, F, G, H, I, L, N, Q, R, S, T, V or Y;
(b) substitution of amino acid S at position 25 with A, D, E, F, I, M, N, T, V or Y;
(c) substitution of amino acid S at position 26 with A, D, E, F, G, H, I, K, M, N, P, R, T or V;
(d) substitution of amino acid Q at position 27 with A, D, E, F, H, I, L, M, S, or Y;
(e) substitution of amino acid S at position 27a with D;
(f) substitution of amino acid L at position 27b with I, P, Q, T or V;
(g) substitution of amino acid F at position 27c with A, D, E, I, M, N, S, W or Y;
(h) substitution of amino acid N at position 27d with A, D, E, F, H, N or Q;
(i) substitution of amino acid S at position 27e with A, D, E, I, L, M, N, P, Q, T, V or Y;
(j) substitution of amino acid R at position 27f with A, E, F, G, H, I, K, L, M, N, Q, S, T, V, W or Y;
(k) substitution of amino acid T at position 28 with E, H, I, Q, S, V, W or Y;
(l) substitution of amino acid R at position 29 with A, D, E, F, G, H, I, K, L, M, N, P, Q, S, T, V, W or Y;
(m) substitution of amino acid K at position 30 with A, D, E, F, H, I, L, M, N, P, Q, S, T, V, W or Y;
(n) substitution of amino acid N at position 31 with D, M, S or T;
(o) substitution of amino acid Y at position 32 with D, E, G, H or Q;
(p) substitution of amino acid Q at position 89 with H, I, T or Y;
(q) substitution of amino acid Y at position 91 with E, F or N;
(r) substitution of amino acid Y at position 93 with I, L, M, N, P, Q, S, T, V or W;
(s) substitution of amino acid H at position 94 with A, D, E, F, G, I, L, M, N, P, Q, R, S, T, V, W or Y;
(t) substitution of amino acid M at position 95 with A, D, E, F, G, H, I, K, L, N, P, Q, R, S, T, V, W or Y;
(u) substitution of amino acid Y at position 96 with A, D, E, F, G, H, K, M, N, P, Q, S, T or V; and
(v) substitution of amino acid T at position 97 with A, D, E, G, H, K, P, Q, R, S or V;
in the amino acid sequence set forth in SEQ ID NO: 4.

In one embodiment, the one or more amino acid substitutions introduced into the light chain variable region of the parent cytosol-penetrating antigen-binding molecule enhance the cytosol-penetrating activity of the antibody or fragment thereof as compared to a reference antibody or fragment thereof without said substitutions and comprising a light chain variable region having the amino acid sequence set forth in SEQ ID NO: 4.

In one embodiment, the one or more amino acid substitutions introduced into the light chain variable region of the parent cytosol-penetrating antigen-binding molecule are selected from the group consisting of (position according to Kabat numbering):
(a) substitution of amino acid K at position 24 with N;
(b) substitution of amino acid Q at position 27 with E;
(c) substitution of amino acid S at position 27a with D;
(d) substitution of amino acid N at position 27d with D or E;
(e) substitution of amino acid S at position 27e with D or E;
(f) substitution of amino acid R at position 27f with D, H, K, L, S or V;
(g) substitution of amino acid T at position 28 with D;
(h) substitution of amino acid R at position 29 with E, G, M or S;
(i) substitution of amino acid K at position 30 with L or Q;
(j) substitution of amino acid Y at position 32 with E;
(k) substitution of amino acid Q at position 89 with H, I, T or Y;
(l) substitution of amino acid Y at position 91 with E, F or N;
(m) substitution of amino acid Y at position 93 with E;
(n) substitution of amino acid H at position 94 with E, P or Q;
(o) substitution of amino acid M at position 95 with D, N or P; and
(p) substitution of amino acid Y at position 96 with E or P;
in the amino acid sequence set forth in SEQ ID NO: 4.

In one embodiment, the one or more amino acid substitutions introduced into the light chain variable region of the parent cytosol-penetrating antigen-binding molecule comprise a combination of amino acid substitutions selected from the group consisting of (position according to Kabat numbering):
(a) S27eD/R27fK;
(b) S27eD/R27fH;
(c) N27dE/S27eD;
(d) S27eD/T28D;
(e) S27eD/R29E;
(f) S27eD/R29S;
(g) K24N/S27eD;
(h) N27dD/R27fK;
(i) N27dE/R27fK;
(j) R27fK/T28D;
(k) R27fK/R29E;
(l) R27fK/R29S;
(m) K24N/R27fK;
(n) K24N/N27dD;
(o) N27dD/T28D;
(p) N27dD/R29E;
(q) N27dD/R29S;
(r) S27eD/Q89H
(s) N27dD/Q89H;
(t) R27fK/Q89H;
(u) S27eD/Y91E;
(v) N27dD/Y91E;
(w) R27fK/Y91E;
(x) Q89H/H94P;
(y) Y91E/H94P;
(z) H94P/M95N;
(aa) H94P/M95D;
(bb) H94P/M95P;
(cc) H94E/Y96P;
(dd) H94E/M95N;
(ee) H94E/M95D;
(ff) H94E/M95P;
(gg) N27dE/R27fK/Q89H/H94E/M95P;
(hh) N27dE/R27fK/Q89Y/H94E/M95P;
(ii) N27dE/R27fK/Y91E/H94E/M95P;
(jj) N27dE/R27fK/Y91F/H94E/M95P;
(kk) N27dE/R27fS/Q89H/H94E/M95P;
(ll) N27dE/R27fS/Q89Y/H94E/M95P;
(mm) N27dE/R27fS/Y91E/H94E/M95P;
(nn) N27dE/R27fS/Y91F/H94E/M95P;
(oo) N27dE/R27fS/Q89H/H94Q/M95P;
(pp) N27dE/R27fS/Q89Y/H94Q/M95P;
(qq) N27dE/R27fS/Y91E/H94Q/M95P;
(rr) N27dE/R27fS/Y91F/H94Q/M95P;
(ss) N27dE/R27fV/Q89H/H94E/M95P;
(tt) N27dE/R27fV/Q89Y/H94E/M95P;
(uu) N27dE/R27fV/Y91E/H94E/M95P;
(vv) N27dE/R27fV/Y91F/H94E/M95P;
(ww) N27dE/R29S/Q89H/H94E/M95P;
(xx) N27dE/R29S/Q89Y/H94E/M95P;
(yy) N27dE/K30L/Q89H/H94E/M95P;
(zz) N27dE/K30L/Q89Y/H94E/M95P;
(aaa) S27eD/R27fK/Y91E/H94E/M95P;
(bbb) S27eD/R27fK/Y91F/H94E/M95P;
(ccc) S27eD/R27fK/Y91E/H94Q/M95P;
(ddd) S27eD/R27fK/Y91F/H94Q/M95P;
(eee) S27eD/R27fK/Y91E/H94Q;
(fff) S27eD/R27fK/Y91F/H94Q;
(ggg) S27eE/R27fK/Y91E/H94E/M95P;
(hhh) S27eE/R27fK/Y91F/H94E/M95P;
(iii) S27eE/R27fK/Y91E/H94Q/M95P;
(jjj) S27eE/R27fK/Y91F/H94Q/M95P;
(kkk) R27fS/R29S/Q89H/H94E/M95P
(lll) R27fS/R29S/Q89Y/H94E/M95P;
(mmm) R27fS/R29S/Y91E/H94E/M95P;
(nnn) R27fS/R29S/Y91F/H94E/M95P;
(ooo) R27fS/R29S/Q89H/H94Q/M95P;
(ppp) R27fS/R29S/Q89Y/H94Q/M95P;
(qqq) R27fS/R29S/Y91E/H94Q/M95P;
(rrr) R27fS/R29S/Y91F/H94Q/M95P;
(sss) N27dE/R27fS/R29S/Q89Y/H94Q/M95P; and
(ttt) N27dE/R27fS/R29S/Y91F/H94Q/M95P.

In one embodiment, the method further comprises replacing the framework region (FR) of the light chain variable region by a human framework region (FR).

In one embodiment, the method further comprises introducing substitution of amino acid L at position 2 with I, and/or substitution of amino acid V at position 3 with Q according to Kabat numbering.

In one preferred embodiment, the FR1 sequence of the light chain variable region is replaced with a human V kappa 1, V kappa 2, or V kappa3 FR1 sequence, more preferably with a human V kappa 1 FR1 sequence. In one preferred embodiment, the FR2 sequence of the light chain variable region is replaced with a human V kappa 1, V kappa 2, or V kappa3 FR2 sequence, more preferably with a human V kappa 1 FR2 sequence. In one preferred embodiment, the FR3 sequence of the light chain variable region is replaced with a human V kappa 1, V kappa 2, or V kappa3 FR3 sequence, more preferably with a human V kappa 3 FR3 sequence. In one embodiment, the light chain variable region comprises a human V kappa 1 FR1, a human V kappa 1 FR2 and a human V kappa 3 FR3 sequences.

In one embodiment, the light chain variable region of the antibody having improved cytosol-penetrating ability comprises one, two, three, or four of the FR domains (FR1, FR2, FR3, and FR4) comprised in SEQ ID NO: 123.

In one embodiment, the method further comprises introducing one, two, three or more amino acid substitutions in the CDRL2 of the light chain variable region of the parent cytosol-penetrating antigen-binding molecule. In one preferred embodiment, the substitutions introduced in the CDRL2 are selected from the group consisting of (position according to Kabat numbering):
(a) substitution of amino acid W at position 50 with A, G, I, T or V;
(b) substitution of amino acid S at position 52 with F or I;
(c) substitution of amino acid T at position 53 with N or Y; and
(d) substitution of amino acid R at position 54 with K or V;
in the CDRL2 of the amino acid sequence set forth in SEQ ID NO: 4.

In one preferred embodiment, the amino acid substitutions introduced into the light chain variable region of the parent cytosol-penetrating antigen-binding molecule comprise a combination of amino acid substitutions selected from the group consisting of (position according to Kabat numbering):
(a) L2I/V3Q/S27eD/R27fK/W50A/Y91F/H94Q;
(b) L2I/V3Q/S27eD/R27fK/W50G/Y91F/H94Q;
(c) L2I/V3Q/S27eD/R27fK/W50I/Y91F/H94Q;
(d) L2I/V3Q/S27eD/R27fK/W50T/Y91F/H94Q;
(e) L2I/V3Q/S27eD/R27fK/W50V/Y91F/H94Q;
(f) L2I/V3Q/S27eD/R27fK/S52F/Y91F/H94Q;
(g) L2I/V3Q/S27eD/R27fK/S52I/Y91F/H94Q;
(h) L2I/V3Q/S27eD/R27fK/T53N/Y91F/H94Q;
(i) L2I/V3Q/S27eD/R27fK/T53Y/Y91F/H94Q;
(j) L2I1/V3Q/S27eD/R27fK/R54K/Y91F/H94Q;
(k) L2I/V3Q/S27eD/R27fK/W50I/S52F/Y91F/H94Q;
(1) L2I/V3Q/S27eD/R27fK/W50I/T53N/Y91F/H94Q; and
(m) L2I/V3Q/S27eD/R27fK/W50I/R54V/Y91F/H94Q;
in the amino acid sequence set forth in SEQ ID NO: 4.

In one embodiment, the cytosol-penetrating antibody or antigen-binding fragment thereof produced comprises a light chain variable region having an amino acid sequence selected from the group consisting of SEQ ID NO: 14-150 and 161-173. In one embodiment, the cytosol-penetrating antibody or antigen-binding fragment thereof produced comprises a light chain variable region having an amino acid sequence selected from the group consisting of SEQ ID NO: 123-150. In one embodiment, the cytosol-penetrating antibody or antigen-binding fragment thereof produced comprises a light chain variable region having an amino acid sequence selected from the group consisting of SEQ ID NO: 161-173. In one embodiment, the cytosol-penetrating antibody or antigen-binding fragment thereof produced is in the form of Fab or scFv. In one embodiment, the cytosol-penetrating antibody or antigen-binding fragment thereof produced further comprises a heavy chain variable region.

### 2. Methods of Screening for a Cytosol-Penetrating Antigen-Binding Molecule

In one aspect, the present disclosure provides methods of screening for a cytosol-penetrating antigen-binding molecule.

In one embodiment, the screening method comprises:
(a) providing a parent cytosol-penetrating antigen-binding molecule comprising a light chain variable region having an amino acid sequence set forth in SEQ ID NO: 4;
(b) introducing one or more amino acid substitutions in at least one selected from CDRL1 and CDRL3 in the light chain variable region of the parent cytosol-penetrating antigen-binding molecule; and
(c) identifying and/or selecting a cytosol-penetrating antibody or antigen-binding fragment thereof comprising the one or more amino acid substitutions which exhibit enhanced cytosol-penetrating ability as compared to the parent cytosol-penetrating antigen-binding molecule.

In one embodiment, the method further comprises:
(c) obtaining an expression vector that comprises a gene encoding the cytosol-penetrating antibody or antigen-binding fragment thereof produced and an operably linked suitable promoter;
(d) introducing the vector into a host cell and culturing the host cell to allow production of the cytosol-penetrating antibody or antigen-binding fragment thereof; and
(e) recovering the cytosol-penetrating antibody or antigen-binding fragment thereof from the host cell culture.

### 3. Methods of Imaging a Cytosolic Antigen

In another aspect, the present disclosure provides methods of imaging a cytosolic antigen comprising using the antigen-binding molecule of the present disclosure, the antigen-binding molecule produced by the above-described production method, or the cytosol-penetrating antigen-binding molecule identified as a suitable molecule by the above-described screening method.

In another aspect, the present disclosure provides the antigen-binding molecule of the present disclosure, the antigen-binding molecule produced by the above-described production method, or the cytosol-penetrating antigen-binding molecule identified as a suitable molecule by the above-described screening method for use in imaging a cytosolic antigen in a sample cell.

In yet another aspect, the present disclosure provides use of the antigen-binding molecule of the present disclosure, the antigen-binding molecule produced by the above-described production method, or the cytosol-penetrating antigen-binding molecule identified as a suitable molecule by the above-described screening method in the manufacture of an agent for imaging a cytosolic antigen.

In certain embodiments of these aspects, the antigen-binding molecule to be used is labeled.

### F. Immunoconjugates

The present disclosure also provides immunoconjugates comprising an antigen-binding molecule herein conjugated to a bioactive molecule. In one embodiment, the bioactive molecule is selected from the group consisting of peptides, proteins, toxins, antibodies, antibody fragments, RNA, DNA, small molecule drugs, nanoparticles and liposomes. In one embodiment, immunoconjugates comprise an antigen-binding molecule herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

G. Methods of Delivering an Antigen-Binding Molecule Specifically into Cytosol of a Target Cell and Methods of Potentiating Cytosol-Penetrating Ability of a Cytosol-Penetrating Antigen-Binding Molecule

In one aspect, the present disclosure provides methods of delivering an antigen-binding molecule specifically into cytosol of a target cell, in which methods a cell surface antigen to which the antigen-binding molecule binds is an antigen expressed specifically on the target cell. In one embodiment, the methods comprise contacting the antigen-binding molecule with the target cell. In one embodiment, the antigen-binding molecule is a multifunctional antigen-binding molecule comprising a cytosol-penetrating antibody or a fragment of the antibody, an antibody binding to a cell surface antigen or a fragment of the antibody, and/or an antibody binding to a cytosolic antigen or a fragment of the antibody in combination. In a certain embodiment, the above-described method substantially does not deliver the antigen-binding molecule to cells that do not express the cell surface antigen. In a certain embodiment, the antigen-binding molecule binds to a cell surface antigen specific to the target cell, and thereby undergoes internalization specifically by the target cell and transfers into the cytosol. In a certain embodiment, the antigen-binding molecule forms a multimer, and displays high cytosol-penetrating ability.

In another aspect, the present disclosure provides methods of potentiating cytosol-penetrating ability of a cytosol-penetrating antigen-binding molecule as compared to a parent cytosol-penetrating antigen-binding molecule, the methods comprising introducing into an Fc region one or more amino acid alterations for enhancing formation of a multimer, the parent cytosol-penetrating antigen-binding molecule comprising a parent Fc region that does not comprise the one or more amino acid alterations.

In another aspect, the present disclosure provides a method of delivering a heterologous moiety specifically into cytosol of a target cell, wherein the heterologous moiety is conjugated to an antigen-binding molecule, which comprises a cell surface antigen-binding domain and a cytosol-penetrating domain. Preferably, the antigen-binding molecule comprises a first and a second Fab regions, wherein (a) the first Fab region binds specifically to a cell surface antigen, and (b) the second Fab region has cytosol-penetrating ability, and wherein the antigen-binding molecule is conjugated to the heterologous moiety. More preferably, the antigen-biding molecule is a bifunctional antibody and does not comprise a cytosolic antigen-binding domain. In one embodiment, the cell surface antigen to which the antigen-binding molecule binds is an antigen expressed specifically on the target cell. In one embodiment, the method comprises contacting said antigen-binding molecule conjugated to the heterologous moiety with the target cell. In a preferred embodiment, the method comprises delivering the heterologous moiety specifically into cytosol of the target cell. In a preferred embodiment, the method substantially does not deliver the heterologous moiety to cells that do not express the cell surface antigen.

### H. Methods for Targeting a Cytosolic Antigen

In a certain embodiment, the antigen-binding molecules of the present disclosure have an elevated ability to remove or suppress a cytosolic antigen in a target cell. Therefore, the antigen-binding molecules provided herein are useful for knocking-down, at protein level, the cytosolic antigen in the target cell. The term “knock(ing) down” used herein means that the amount of expression or abundance of a certain protein is reduced. Specifically, when a cytosolic antigen is knocked-down at protein level, the amount of the cytosolic antigen per cell is reduced. In another certain embodiment, the antigen-binding molecules of the present disclosure have an elevated ability to activate a cytosolic antigen in a target cell.

### I. Methods and Compositions for Diagnosis and Detection

In a certain embodiment, the antigen-binding molecules provided herein are useful in detecting the presence of a cytosolic antigen in a target cell in a biological sample. The term “detection/detecting” used herein encompasses quantitative or qualitative detection.

In one embodiment, the antigen-binding molecules for use in the methods of diagnosis or the methods of detection are provided. In a further aspect, methods of detecting the presence of a cytosolic antigen in a target cell in a biological sample are provided. In a certain embodiment, the method comprises allowing an antigen-binding molecule described herein with a biological sample under the conditions permissible for the binding of the antigen-binding molecule to a cytosolic antigen, and detecting if a complex is formed between the antigen-binding molecule and the cytosolic antigen. Such methods may be in vitro methods or in vivo methods. In a different embodiment, the antigen-binding molecules of the present disclosure are useful for the detection of a cytosolic antigen by imaging or such methods.

In certain embodiments, labeled antigen-binding molecules of the present disclosure are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵, 3H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, those coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### J. Pharmaceutical Formulations

Pharmaceutical formulations of an antigen-binding molecule as described herein are prepared by mixing such antigen-binding molecule having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antigen-binding molecule, which matrices are in the form of shaped articles, e.g. films, or microcapsules.

The formulations to be used for in vivo administration are generally sterile. Sterility may be readily accomplished, e.g., by filtration through sterile filtration membranes.

### K. Therapeutic Methods and Compositions

In one aspect, the antigen-binding molecules provided herein may be used in therapeutic methods.

In one embodiment, the antigen-binding molecules of the present disclosure for use as pharmaceuticals are provided. In certain embodiments, the antigen-binding molecules of the present disclosure for use in methods of treatment are provided. In one of these embodiments, the method further comprises a step of administering an effective amount of at least one additional therapeutic agent (e.g., such as those described below) to the subject. The “subject” according to any of the above-described embodiments is preferably a human.

In a further aspect, the present disclosure provides use of the antigen-binding molecules of the present disclosure in the manufacture or preparation of medicaments. In one of such embodiments, the method further comprises a step of administering an effective amount of at least one additional therapeutic agent (e.g., such as those described below) to the subject. The “subject” according to any of the above-described embodiments may be a human.

In a further aspect, the present disclosure provides pharmaceutical formulations comprising any of the antigen-binding molecules provided herein, e.g., for use in any of the above therapeutic methods. In one embodiment, a pharmaceutical formulation comprises any of the antigen-binding molecules provided herein and a pharmaceutically acceptable carrier. In another embodiment, a pharmaceutical formulation comprises any of the antigen-binding molecules provided herein and at least one additional therapeutic agent.

An antigen-binding molecule of the present disclosure (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antigen-binding molecules of the present disclosure would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antigen-binding molecule need not be, but is optionally formulated with one or more agents currently used to prevent or treat the disorder in question. The effective amount of such other agents depends on the amount of antigen-binding molecule present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antigen-binding molecule of the present disclosure (when used alone or in combination with one or more other additional therapeutic agents) will depend on the type of disease to be treated, the type of antibody when the antigen-binding molecule of the present disclosure is an antibody, the severity and course of the disease, whether the antigen-binding molecule is administered for preventive or therapeutic purposes, previous therapy, the patient's clinical history and response to the antigen-binding molecule, and the discretion of the attending physician. The antigen-binding molecule is suitably administered to the patient at one time or over a series of treatments. Depending on the type and severity of the disease, about 1 micro g/kg to 15 mg/kg (e.g. 0.1 mg/kg-10 mg/kg) of antigen-binding molecule can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the antigen-binding molecule would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antigen-binding molecule). An initial higher loading dose, followed by one or more lower doses may be administered. The progress of this therapy is easily monitored by conventional techniques and assays.

It is understood that any of the above formulations or therapeutic methods may be carried out using an immunoconjugate of the present disclosure in place of or in addition to an antigen-binding molecule.

### L. Articles of Manufacture

In another aspect of the present disclosure, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antigen-binding molecule of the present disclosure. The label or package insert indicates that the composition is used for treating the condition of choice. Moreover, the article of manufacture may comprise (a) a first container with a composition contained therein, wherein the composition comprises an antigen-binding molecule of the present disclosure; and (b) a second container with a composition contained therein, wherein the composition comprises a further cytotoxic or otherwise therapeutic agent. The article of manufacture in this embodiment of the present disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the present disclosure in place of or in addition to an antigen-binding molecule.

### Examples

The following are examples of methods and compositions of the present disclosure. It is understood that various other embodiments may be practiced, given the general description provided above.

Although the foregoing invention has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

### Example 1: Comprehensive Mutagenesis Approach to Improve Cytosol Trafficking Ability and Antibody Expression Level of Parental Light Chain Variable Region (VL) hT4VL.FRv4

The present invention provided cytosol-penetrating antibodies, in particular light chain variable regions (VL) with improved cytoplasmic permeability over a previously reported cytosol-penetrating antibody h3D8, in particular the VL of h3D8 (i.e. hT4VL) disclosed in WO2016013870; and confirmed that the antigen-binding molecules or antibodies have improved permeability to the cytoplasm.

The present inventors first converted each framework (FR) of hT4VL (SEQ ID NO: 174) to human V kappa 1 or V kappa 3 germline sequences as listed in Table 2. As a result, hT4VL.FRv4 (SEQ ID NO: 4) which only differs from hT4VL by having a human V kappa 3 (VK3) FR3 was eventually selected as the best VL candidate in terms of a combination of expression yield, aggregation profile revealed by single SEC peak, and cytosol-penetrating activity for further improvement. Subsequently, hT4VL.FRv4 (SEQ ID NO: 4) was selected as a parent VL to comprehensively introduce amino acid modifications other than cysteine into all the positions in CDRL1 and CDRL3 to find mutations which can improve cytosol trafficking ability and antibody expression level. The genes encoding the VL variants were combined with light chain constant region (KT0, SEQ ID NO: 12) and transiently expressed using Expi293 cell line (Thermo Fisher, Carlsbad, CA, USA) together with heavy chain expression vector (3D8H-SG1. SmBiTb, SEQ ID NO: 13). Expression and purification was carried out by methods known to those skilled in the art. Variant's expression level was summarized in Table 3. The variants with improved expression level compared to the parental antibody having hT4VL.FRv4 were shown in bold. To assess cytosol trafficking ability, the present inventors carried out Split Nanoluc (Split Nluc) assay as described in Reference Example 1.

TABLE 2
List of framework variants of hT4VL.

| Design Name | FR1 | FR2 | FR3 | SEQ ID NO. |
|---|---|---|---|---|
| hT4VL.FRv1 | VK3 | VK1 | VK1 | 1 |
| hT4VL.FRv2 | VK3 (I2L, L4M) | VK1 | VK1 | 2 |
| hT4VL.FRv3 | VK1 (I2L, Q3V) | VK3 | VK1 | 3 |
| hT4VL.FRv4 | VK1 (I2L, Q3V) | VK1 | VK3 | 4 |
| hT4VL.FRv5 | VK3 | VK3 | VK1 | 5 |
| hT4VL.FRv6 | VK3 (I2L, L4M) | VK3 | VK1 | 6 |
| hT4VL.FRv7 | VK3 | VK1 | VK3 | 7 |
| hT4VL.FRv8 | VK3 (I2L, L4M) | VK1 | VK3 | 8 |
| hT4VL.FRv9 | VK3 | VK3 | VK3 | 9 |
| hT4VL.FRv10 | VK3 (I2L, L4M) | VK3 | VK3 | 10 |
| hT4VL.FRv11 | VK1 (I2L, Q3V) | VK3 | VK3 | 11 |
| hT4VL | VK1 (I2L, Q3V) | VK1 | VK1 | 174 |

TABLE 3
Expression level of each mutated variant (mg/mL)

| Orig- | | | | | | | | | | | | | | | | | | | | |
|---|---|---|---|---|---|---|---|---|---|---|---|---|---|---|---|---|---|---|---|---|
| inal | | A | D | E | F | G | H | I | K | L | M | N | P | Q | R | S | T | V | W | Y |
| K | 24 | 0.24 | 0.17 | 0.27 | 0.32 | 0.22 | 0.36 | 0.39 | | 0.39 | 0.31 | 0.67 | 0.10 | 0.46 | 0.40 | 0.45 | 0.52 | 0.27 | 0.20 | 0.40 |
| S | 25 | 0.24 | 0.17 | 0.20 | 0.44 | 0.29 | 0.23 | 0.33 | 0.15 | 0.20 | 0.29 | 0.11 | 0.25 | 0.17 | 0.08 | | 0.39 | 0.44 | 0.16 | 0.29 |
| S | 26 | 0.41 | 0.47 | 0.44 | 0.23 | 0.46 | 0.36 | 0.11 | 0.33 | 0.20 | 0.34 | 0.57 | 0.41 | 0.32 | 0.36 | | 0.39 | 0.26 | 0.18 | 0.20 |
| Q | 27 | 0.44 | 0.54 | 0.72 | 0.43 | 0.44 | 0.41 | 0.42 | 0.33 | 0.44 | 0.45 | 0.36 | 0.16 | | 0.26 | 0.52 | 0.32 | 0.44 | 0.35 | 0.41 |
| S | 27a | 0.33 | 0.73 | 0.48 | 0.21 | 0.37 | 0.24 | 0.26 | 0.17 | 0.29 | 0.39 | 0.49 | 0.33 | 0.35 | 0.10 | | 0.22 | 0.31 | 0.19 | 0.29 |
| L | 27b | 0.18 | 0.25 | 0.21 | 0.16 | 0.07 | 0.08 | 0.29 | 0.07 | | 0.21 | 0.09 | 0.16 | 0.15 | 0.07 | 0.08 | 0.11 | 0.40 | 0.06 | 0.10 |
| F | 27c | 0.18 | 0.21 | 0.55 | | 0.17 | 0.30 | 0.30 | 0.03 | 0.35 | 0.35 | 0.30 | 0.15 | 0.29 | 0.07 | 0.23 | 0.19 | 0.24 | 0.45 | 0.33 |
| N | 27d | 0.19 | 0.83 | 0.84 | 0.26 | 0.27 | 0.50 | 0.20 | 0.13 | 0.30 | 0.29 | | 0.17 | 0.40 | 0.11 | 0.39 | 0.13 | 0.41 | 0.29 | 0.27 |
| S | 27e | 0.49 | 1.01 | 0.82 | 0.40 | 0.44 | 0.35 | 0.43 | 0.13 | 0.52 | 0.52 | 1.00 | 0.44 | 0.60 | 0.17 | | 0.61 | 0.58 | 0.28 | 0.41 |
| R | 27f | 1.04 | 0.98 | 1.02 | 0.95 | 1.03 | 1.10 | 0.98 | 0.85 | 1.00 | 0.89 | 0.94 | 0.62 | 0.83 | | 1.00 | 0.97 | 1.05 | 0.63 | 0.74 |
| T | 28 | 0.33 | 0.87 | 0.30 | 0.39 | 0.26 | 0.29 | 0.46 | 0.11 | 0.45 | 0.37 | 0.35 | 0.15 | 0.47 | 0.17 | 0.30 | | 0.42 | 0.31 | 0.36 |
| R | 29 | 1.04 | 0.99 | 0.86 | 0.67 | 1.17 | 0.70 | 0.46 | 0.46 | 0.87 | 0.99 | 0.87 | 0.41 | 0.65 | | 0.80 | 0.86 | 0.57 | 0.55 | 0.82 |
| K | 30 | 0.85 | 0.73 | 0.67 | 0.65 | 0.44 | 0.64 | 0.72 | | 0.96 | 0.81 | 0.74 | 0.43 | 1.00 | 0.41 | 0.42 | 0.91 | 0.73 | 0.31 | 0.80 |
| N | 31 | 0.48 | 0.68 | 0.43 | 0.17 | 0.19 | 0.45 | 0.18 | 0.07 | 0.12 | 0.23 | | 0.22 | 0.19 | 0.09 | 0.43 | 0.43 | 0.23 | 0.28 | 0.39 |
| Y | 32 | 0.46 | 0.88 | 1.07 | 0.48 | 0.49 | 0.69 | 0.28 | 0.03 | 0.59 | 0.59 | 0.59 | 0.30 | 0.57 | 0.25 | 0.56 | 0.37 | 0.27 | 0.27 | |
| L | 33 | 0.23 | 0.16 | 0.17 | 0.18 | 0.14 | 0.12 | 0.40 | 0.05 | | 0.23 | 0.18 | 0.15 | 0.29 | 0.14 | 0.14 | 0.29 | 0.35 | 0.15 | 0.08 |
| A | 34 | | 0.15 | 0.09 | 0.20 | 0.30 | 0.25 | 0.13 | 0.32 | 0.18 | 0.13 | 0.12 | 0.12 | 0.06 | 0.09 | 0.33 | 0.19 | 0.12 | 0.08 | 0.41 |
| Q | 89 | 0.32 | 0.36 | 0.12 | 0.61 | 0.31 | 0.86 | 0.55 | 0.47 | 0.33 | 0.73 | 0.53 | 0.22 | | 0.10 | 0.62 | 0.67 | 0.66 | 0.55 | 0.83 |
| Q | 90 | 0.46 | 0.54 | 0.63 | 0.38 | 0.26 | 0.56 | 0.38 | 0.22 | 0.27 | 0.34 | 0.49 | 0.59 | | 0.23 | 0.47 | 0.41 | 0.34 | 0.29 | 0.39 |
| Y | 91 | 0.52 | 0.46 | 0.69 | 0.65 | 0.52 | 0.74 | 0.45 | 0.11 | 0.61 | 0.76 | 0.70 | 0.42 | 0.65 | 0.08 | 0.72 | 0.63 | 0.04 | 0.65 | |
| Y | 92 | 0.67 | 0.61 | 0.58 | 0.47 | 0.49 | 0.32 | 0.41 | 0.18 | 0.53 | 0.56 | 0.65 | 0.76 | 0.43 | 0.13 | 0.16 | 0.45 | 0.35 | 0.36 | |
| Y | 93 | 0.62 | 0.95 | 0.97 | 0.50 | 0.61 | 0.27 | 0.49 | 0.21 | 0.50 | 0.59 | 0.76 | 0.78 | 0.64 | 0.22 | 0.63 | 0.75 | 0.62 | 0.40 | |
| H | 94 | 0.93 | 1.19 | 1.12 | 0.80 | 0.92 | | 0.59 | 0.27 | 0.62 | 0.87 | 0.26 | 1.18 | 1.03 | 0.37 | 1.01 | 0.87 | 0.68 | 0.85 | 0.77 |
| M | 95 | 0.74 | 1.01 | 0.85 | 0.56 | 1.04 | 0.61 | 0.49 | 0.48 | 0.62 | | 1.06 | 1.17 | 0.78 | 0.57 | 0.67 | 0.61 | 0.57 | 0.63 | 0.53 |
| Y | 96 | 0.44 | 0.75 | 0.88 | 0.70 | 0.71 | 0.54 | 0.46 | 0.57 | 0.45 | 0.79 | 0.55 | 0.92 | 0.87 | 0.37 | 0.78 | 0.92 | 0.48 | 0.31 | |
| T | 97 | 0.55 | 0.77 | 0.62 | 0.41 | 0.55 | 0.54 | 0.42 | 0.51 | 0.47 | 0.47 | 0.61 | 0.64 | 0.48 | 0.53 | 0.52 | | 0.41 | 0.45 | 0.47 |
| Pa- | | 0.53 | | | | | | | | | | | | | | | | | | |
| ren- | | | | | | | | | | | | | | | | | | | | |
| tal | | | | | | | | | | | | | | | | | | | | |
| Ab | | | | | | | | | | | | | | | | | | | | |

TABLE 4
Cytosol trafficking ability assessed by Split Nanoluc assay (Fold change with reference to a buffer well)

| Original | | A | D | E | F | G | H | I | K | L | M | N | P | Q | R | S | T | V | W | Y |
|---|---|---|---|---|---|---|---|---|---|---|---|---|---|---|---|---|---|---|---|---|
| K | 24 | 5.5 | 6.5 | 6.1 | 6.3 | 5.4 | 6.5 | 6.0 | | 5.6 | 5.0 | 8.8 | 3.3 | 6.3 | 5.9 | 5.3 | 5.4 | 5.9 | 4.0 | 6.9 |
| S | 25 | 5.1 | 5.3 | 5.2 | 5.4 | 4.9 | 4.0 | 5.0 | 2.6 | 4.7 | 5.3 | 6.1 | 4.6 | 3.7 | 3.5 | | 5.4 | 6.0 | 4.6 | 5.4 |
| S | 26 | 5.6 | 5.7 | 5.9 | 6.1 | 5.2 | 5.7 | 6.8 | 5.1 | 4.0 | 5.0 | 6.8 | 6.2 | 4.7 | 6.1 | | 5.5 | 5.5 | 4.0 | 4.1 |
| Q | 27 | 5.3 | 7.2 | 5.7 | 5.9 | 4.4 | 5.2 | 5.2 | 4.4 | 6.3 | 6.3 | 4.6 | 4.3 | | 3.7 | 5.3 | 4.1 | 4.9 | 4.9 | 5.9 |
| S | 27a | 4.5 | 6.8 | 4.7 | 3.3 | 3.6 | 3.3 | 4.7 | 4.2 | 4.2 | 4.7 | 4.6 | 3.9 | 4.2 | 2.5 | | 3.9 | 3.9 | 3.5 | 4.7 |
| L | 27b | 3.6 | 4.3 | 3.7 | 2.4 | 1.9 | 2.3 | 6.3 | 3.1 | | 4.7 | 4.5 | 6.1 | 5.5 | 4.3 | 4.2 | 5.7 | 7.5 | 3.6 | 4.0 |
| F | 27c | 6.2 | 5.4 | 6.6 | | 4.9 | 4.6 | 6.4 | 1.5 | 4.4 | 5.3 | 5.1 | 4.0 | 4.6 | 3.6 | 5.8 | 4.8 | 4.4 | 5.7 | 5.4 |
| N | 27d | 5.1 | 9.2 | 6.5 | 5.8 | 4.0 | 5.2 | 4.5 | 3.5 | 4.4 | 5.1 | | 4.2 | 6.7 | 3.9 | 4.4 | 3.3 | 4.9 | 4.6 | 4.5 |
| S | 27e | 6.9 | 11.0 | 7.0 | 5.0 | 4.8 | 4.4 | 5.7 | 3.6 | 6.5 | 8.9 | 6.1 | 5.1 | 5.4 | 2.8 | | 6.6 | 7.0 | 4.3 | 6.3 |
| R | 27f | 6.6 | 4.4 | 5.1 | 7.1 | 6.6 | 8.3 | 7.3 | 10.4 | 7.8 | 5.5 | 5.1 | 4.8 | 6.3 | | 6.7 | 5.4 | 7.0 | 7.6 | 5.5 |
| T | 28 | 3.4 | 4.8 | 5.2 | 4.8 | 4.4 | 6.2 | 5.2 | 2.7 | 4.4 | 3.6 | 4.6 | 3.7 | 5.3 | 2.8 | 5.1 | | 7.0 | 5.2 | 5.7 |
| R | 29 | 6.7 | 7.2 | 5.3 | 8.4 | 8.2 | 6.8 | 5.1 | 6.3 | 7.0 | 7.9 | 7.4 | 6.2 | 6.6 | | 7.7 | 6.2 | 5.4 | 5.5 | 7.3 |
| K | 30 | 6.6 | 6.2 | 5.0 | 7.4 | 4.4 | 5.2 | 5.8 | | 7.2 | 6.6 | 6.6 | 5.6 | 8.6 | 4.6 | 5.1 | 7.0 | 7.4 | 6.0 | 6.7 |
| N | 31 | 4.9 | 6.5 | 4.0 | 3.7 | 3.4 | 5.0 | 4.6 | 3.8 | 4.1 | 5.6 | | 3.3 | 3.3 | 2.7 | 5.3 | 6.1 | 4.7 | 4.1 | 4.5 |
| Y | 32 | 4.8 | 5.5 | 5.9 | 4.7 | 5.2 | 5.6 | 4.2 | 2.1 | 4.1 | 4.3 | 4.8 | 4.2 | 5.7 | 3.7 | 4.6 | 4.5 | 3.0 | 3.2 | |
| L | 33 | 3.3 | 3.4 | 3.5 | 4.2 | 3.2 | 3.0 | 4.0 | 1.9 | | 3.6 | 3.3 | 3.2 | 4.6 | 1.8 | 3.4 | 3.4 | 3.2 | 3.2 | 2.5 |
| A | 34 | | 4.1 | 2.7 | 2.8 | 3.6 | 3.8 | 3.1 | 4.0 | 3.6 | 3.4 | 2.7 | 3.7 | 2.9 | 3.2 | 3.2 | 3.6 | 3.9 | 2.9 | 2.8 |
| Q | 89 | 3.2 | 3.9 | 3.1 | 3.4 | 3.3 | 4.4 | 3.9 | 3.5 | 3.3 | 3.6 | 3.2 | 3.0 | | 2.7 | 3.2 | 4.3 | 3.5 | 3.2 | 4.0 |
| Q | 90 | 3.2 | 3.2 | 3.3 | 3.5 | 3.8 | 3.3 | 3.3 | 3.0 | 2.6 | 2.9 | 3.5 | 3.2 | | 3.5 | 3.2 | 3.1 | 3.0 | 2.9 | 3.0 |
| Y | 91 | 3.1 | 3.1 | 4.3 | 3.4 | 3.1 | 3.0 | 2.8 | 2.6 | 3.1 | 3.1 | 3.7 | 2.9 | 3.0 | 2.2 | 3.0 | 3.0 | 1.7 | 2.8 | |
| Y | 92 | 3.5 | 3.0 | 2.6 | 2.7 | 2.7 | 2.3 | 2.6 | 2.3 | 3.7 | 2.9 | 2.4 | 2.9 | 2.5 | 1.9 | 2.4 | 2.8 | 2.8 | 2.5 | |
| Y | 93 | 2.5 | 3.3 | 3.3 | 2.5 | 4.9 | 3.7 | 6.2 | 3.6 | 6.0 | 6.8 | 6.8 | 8.7 | 6.4 | 3.9 | 7.1 | 6.4 | 5.9 | 5.5 | |
| H | 94 | 8.5 | 9.3 | 8.5 | 7.2 | 8.8 | | 6.8 | 4.6 | 7.6 | 9.0 | 6.3 | 10.5 | 9.4 | 5.1 | 9.4 | 8.0 | 7.1 | 8.0 | 7.5 |
| M | 95 | 6.7 | 8.1 | 7.1 | 6.1 | 7.1 | 6.3 | 5.6 | 5.4 | 6.0 | | 8.6 | 7.0 | 6.4 | 5.4 | 6.6 | 6.1 | 5.7 | 6.0 | 6.1 |
| Y | 96 | 5.9 | 6.8 | 7.6 | 6.8 | 6.0 | 5.0 | 4.7 | 5.1 | 4.3 | 6.9 | 5.0 | 8.4 | 6.6 | 4.9 | 6.4 | 7.5 | 5.1 | 4.9 | |
| T | 97 | 5.2 | 6.9 | 5.4 | 4.5 | 5.3 | 5.7 | 4.7 | 5.2 | 4.9 | 4.6 | 4.5 | 5.4 | 5.3 | 5.0 | 5.4 | | 5.2 | 4.5 | 4.2 |
| Parental Ab | | 5.0 | | | | | | | | | | | | | | | | | | |

### Example 2: Variants with Amino Acid Mutation(s) which Improve Cytosol-Penetrating and Other Properties of hT4VL.FRv4

Based on the results of Example 1, multiple variants were selected to introduce further amino acid mutations (Table 5) and tested Ab expression level, cytosol trafficking ability and extracellular matrix (ECM) binding. Antibodies with low ECM binding were considered as more favorable as high ECM binding may worsen the antibody pharmacokinetics (US2014/0080153). ECM binding studies were performed as described in Reference Example 2. The results were summarized in Table 5 and Tables 6-1 and 6-2. The variants with better antibody expression level, higher split Nanoluc signal and lower ECM binding compared to those of the parental antibody hT4VL.FRv4 were shown in bold. Acidic residues (D or E) seem to be better at position 27, 27a, 27d, 27e, and 28 (Kabat numbering), Lysine or Serine at position 27f (Kabat numbering), and Proline or Glutamine at position 94 (Kabat numbering) seem to be promising amino acid residues in respective positions. Importantly, most of the combination variants listed in Tables 6-1 and 6-2 showed better profile than the parental Ab (hT4VL.FRv4). Especially, mutation combinations S27eD/R27fK, N27dE/R27fK, N27dD/Q89H, R27fK/Y91E, H94E/M95P, N27dE/R27fK/Q89Y/H94E/M95P, N27dE/R27fK/Y91F/H94E/M95P, N27dE/R27fS/Q89Y/H94E/M95P, N27dE/R27fS/Q89Y/H94Q/M95P, S27eD/R27fK/Y91F/H94Q/M95P, S27eD/R27fK/Y91F/H94Q, and S27eE/R27fK/Y91F/H94Q/M95P (Kabat numbering) showed higher Split Nluc signal compared to other combinations while showing low ECM binding.

TABLE 5
Selected variants single amino acid mutation

| | | Ab | Split Nluc | ECM | ECM | |
|---|---|---|---|---|---|---|
| | | expression | (Fold | binding vs | binding vs | SEQ |
| | | level | change vs | Control Ab | Control Ab | ID |
| Domain name | Mutation | (mg/mL) | parent Ab) | (pH 7.4) | (pH 5.8) | NO |
| hT4VL.FRv40010 | K24N | 0.57 | 1.45 | 8.4 | 102.3 | 14 |
| hT4VL.FRv40057 | Q27E | 0.66 | 1.35 | 9.0 | 103.4 | 15 |
| hT4VL.FRv40074 | S27aD | 0.52 | 1.29 | 6.8 | 96.1 | 16 |
| hT4VL.FRv40128 | N27dD | 0.91 | 1.43 | 4.7 | 36.3 | 17 |
| hT4VL.FRv40129 | N27dE | 1.03 | 1.52 | 6.7 | 59.3 | 18 |
| hT4VL.FRv40146 | S27eD | 1.37 | 1.39 | 5.3 | 61.6 | 19 |
| hT4VL.FRv40147 | S27eE | 1.34 | 1.42 | 5.6 | 75.8 | 20 |
| hT4VL.FRv40164 | R27fD | 1.29 | 1.24 | 1.3 | 12.3 | 21 |
| hT4VL.FRv40168 | R27fH | 1.32 | 1.34 | 5.1 | 97.0 | 22 |
| hT4VL.FRv40170 | R27fK | 0.93 | 1.31 | 8.5 | 92.9 | 23 |
| hT4VL.FRv40171 | R27fL | 1.13 | 1.40 | 2.9 | 28.1 | 24 |
| hT4VL.FRv40176 | R27fS | 1.14 | 1.56 | 3.3 | 35.4 | 25 |
| hT4VL.FRv40178 | R27fV | 1.03 | 1.51 | 2.6 | 27.7 | 26 |
| hT4VL.FRv40182 | T28D | 1.19 | 1.46 | 5.2 | 34.5 | 27 |
| hT4VL.FRv40201 | R29E | 1.06 | 1.27 | 3.5 | 58.2 | 28 |
| hT4VL.FRv40203 | R29G | 1.15 | 1.46 | 5.3 | 90.2 | 29 |
| hT4VL.FRv40208 | R29M | 0.78 | 1.33 | 5.2 | 80.1 | 30 |
| hT4VL.FRv40212 | R29S | 1.16 | 1.52 | 6.1 | 93.9 | 31 |
| hT4VL.FRv40224 | K30L | 0.48 | 1.45 | 8.6 | 76.9 | 32 |
| hT4VL.FRv40228 | K30Q | 0.73 | 1.23 | 7.7 | 63.7 | 33 |
| hT4VL.FRv40255 | Y32E | 0.71 | 1.30 | 4.5 | 31.3 | 34 |
| hT4VL.FRv40312 | Q89H | 0.79 | 1.16 | 14.0 | 100.3 | 35 |
| hT4VL.FRv40313 | Q89I | 0.44 | 1.02 | 14.5 | 102.0 | 36 |
| hT4VL.FRv40321 | Q89T | 0.45 | 1.14 | 19.3 | 101.9 | 37 |
| hT4VL.FRv40324 | Q89Y | 0.72 | 1.29 | 14.0 | 98.1 | 38 |
| hT4VL.FRv40345 | Y91E | 0.95 | 1.15 | 9.6 | 97.2 | 39 |
| hT4VL.FRv40346 | Y91F | 0.53 | 1.30 | 16.3 | 99.2 | 40 |
| hT4VL.FRv40353 | Y91N | 0.54 | 1.21 | 15.2 | 101.4 | 41 |
| hT4VL.FRv40381 | Y93E | 0.94 | 1.36 | 7.3 | 97.4 | 42 |
| hT4VL.FRv40399 | H94E | 0.98 | 1.40 | 8.6 | 53.1 | 43 |
| hT4VL.FRv40407 | H94P | 1.01 | 1.28 | 13.1 | 81.3 | 44 |
| hT4VL.FRv40408 | H94Q | 0.94 | 1.44 | 15.9 | 93.8 | 45 |
| hT4VL.FRv40416 | M95D | 0.80 | 1.29 | 10.4 | 95.9 | 46 |
| hT4VL.FRv40424 | M95N | 0.66 | 1.35 | 11.0 | 98.2 | 47 |
| hT4VL.FRv40425 | M95P | 0.77 | 1.25 | 12.1 | 98.5 | 48 |
| hT4VL.FRv40435 | Y96E | 0.95 | 1.24 | 15.0 | 97.8 | 49 |
| hT4VL.FRv40444 | Y96P | 0.71 | 1.26 | 14.9 | 96.4 | 50 |
| hT4VL.FRv4 | parent | 0.51 | 1.00 | 15.5 | 99.1 | 4 |

TABLE 6-1
Variants with two or more amino acid mutation combination

| | | Ab | Split Nluc | ECM | ECM | |
|---|---|---|---|---|---|---|
| | | expression | (Fold | binding vs | binding vs | SEQ |
| | | level | change vs | Control Ab | Control Ab | ID |
| Domain name | Mutation | (mg/mL) | parent Ab) | (pH 7.4) | (pH 5.8) | NO |
| hT4VL.FRv40469 | S27eD, R27fK | 0.40 | 1.41 | 3.34 | 33.93 | 51 |
| hT4VL.FRv40470 | S27eD, R27fH | 0.84 | 1.15 | 2.85 | 49.64 | 52 |
| hT4VL.FRv40471 | N27dE, S27eD | 0.86 | 1.18 | 2.50 | 18.21 | 53 |
| hT4VL.FRv40472 | S27eD, T28D | 0.82 | 1.07 | 1.97 | 10.01 | 54 |
| hT4VL.FRv40473 | S27eD, R29E | 0.77 | 1.15 | 1.46 | 15.09 | 55 |
| hT4VL.FRv40474 | S27eD, R29S | 1.44 | 1.16 | 2.69 | 25.90 | 56 |
| hT4VL.FRv40475 | K24N, S27eD | 1.08 | 1.19 | 3.84 | 49.04 | 57 |
| hT4VL.FRv40476 | N27dD, R27fK | 0.81 | 1.15 | 2.95 | 17.91 | 58 |
| hT4VL.FRv40477 | N27dE, R27fK | 0.82 | 1.31 | 3.73 | 30.43 | 59 |
| hT4VL.FRv40478 | R27fK, T28D | 0.85 | 1.23 | 3.41 | 18.57 | 60 |
| hT4VL.FRv40479 | R27fK, R29E | 1.06 | 1.23 | 1.90 | 18.35 | 61 |
| hT4VL.FRv40480 | R27fK, R29S | 0.68 | 1.26 | 3.38 | 42.51 | 62 |
| hT4VL.FRv40481 | K24N, R27fK | 0.80 | 1.19 | 6.21 | 73.82 | 63 |
| hT4VL.FRv40482 | K24N, N27dD | 0.94 | 1.16 | 2.83 | 22.08 | 64 |
| hT4VL.FRv40483 | N27dD, T28D | 1.04 | 1.22 | 2.11 | 11.05 | 65 |
| hT4VL.FRv40484 | N27dD, R29E | 1.31 | 0.98 | 1.07 | 10.43 | 66 |
| hT4VL.FRv40485 | N27dD, R29S | 1.29 | 1.27 | 2.36 | 13.09 | 67 |
| hT4VL.FRv40486 | S27eD, Q89H | 1.16 | 1.17 | 6.00 | 46.28 | 68 |
| hT4VL.FRv40487 | N27dD, Q89H | 0.72 | 1.40 | 6.94 | 31.78 | 69 |
| hT4VL.FRv40488 | R27fK, Q89H | 0.70 | 1.20 | 9.74 | 74.48 | 70 |
| hT4VL.FRv40489 | S27eD, Y91E | 1.08 | 1.26 | 3.92 | 53.76 | 71 |
| hT4VL.FRv40490 | N27dD, Y91E | 1.67 | 1.21 | 4.37 | 33.26 | 72 |
| hT4VL.FRv40491 | R27fK, Y91E | 0.98 | 1.32 | 6.17 | 69.49 | 73 |
| hT4VL.FRv40492 | Q89H, H94P | 1.17 | 1.28 | 12.43 | 64.97 | 74 |
| hT4VL.FRv40493 | Y91E, H94P | 1.42 | 1.22 | 11.25 | 66.06 | 75 |
| hT4VL.FRv40494 | H94P, M95N | 1.15 | 1.21 | 10.37 | 59.58 | 76 |
| hT4VL.FRv40495 | H94P, M95D | 1.04 | 1.11 | 12.88 | 47.53 | 77 |
| hT4VL.FRv40496 | H94P, M95P | 1.12 | 1.16 | 10.82 | 62.82 | 78 |
| hT4VL.FRv40497 | H94E, Y96P | 1.06 | 1.20 | 8.21 | 37.46 | 79 |
| hT4VL.FRv40498 | H94E, M95N | 1.06 | 1.28 | 7.71 | 44.04 | 80 |
| hT4VL.FRv40499 | H94E, M95D | 1.33 | 1.30 | 6.90 | 41.94 | 81 |
| hT4VL.FRv40500 | H94E, M95P | 1.10 | 1.41 | 7.03 | 60.03 | 82 |
| hT4VL.FRv4 | parent | 0.63 | 1.00 | 13.05 | 71.42 | 4 |

TABLE 6-2
Variants with two or more amino acid mutation combination

| | | Ab | Split Nluc | ECM | ECM | |
|---|---|---|---|---|---|---|
| | | expression | (Fold | binding vs | binding vs | SEQ |
| | | level | change vs | Control Ab | Control Ab | ID |
| Domain name | Mutation | (mg/mL) | parent Ab) | (pH 7.4) | (pH 5.8) | NO |
| hT4VL.FRv40501 | N27dE, R27fK, Q89H, | 0.80 | 1.41 | 1.04 | 7.52 | 83 |
| | H94E, M95P | | | | | |
| hT4VL.FRv40502 | N27dE, R27fK, Q89Y, | 0.74 | 1.77 | 1.65 | 19.26 | 84 |
| | H94E, M95P | | | | | |
| hT4VL.FRv40503 | N27dE, R27fK, Y91E, | 0.89 | 1.37 | 1.10 | 14.14 | 85 |
| | H94E, M95P | | | | | |
| hT4VL.FRv40504 | N27dE, R27fK, Y91F, | 0.92 | 1.41 | 1.57 | 12.86 | 86 |
| | H94E, M95P | | | | | |
| hT4VL.FRv40505 | N27dE, R27fS, Q89H, | 0.87 | 1.08 | 0.66 | 4.21 | 87 |
| | H94E, M95P | | | | | |
| hT4VL.FRv40506 | N27dE, R27fS, Q89Y, | 0.97 | 1.36 | 1.04 | 11.59 | 88 |
| | H94E, M95P | | | | | |
| hT4VL.FRv40507 | N27dE, R27fS, Y91E, | 1.09 | 0.86 | 0.77 | 8.26 | 89 |
| | H94E, M95P | | | | | |
| hT4VL.FRv40508 | N27dE, R27fS, Y91F, | 0.96 | 1.14 | 0.78 | 5.73 | 90 |
| | H94E, M95P | | | | | |
| hT4VL.FRv40509 | N27dE, R27fS, Q89H, | 0.99 | 1.38 | 1.02 | 6.26 | 91 |
| | H94Q, M95P | | | | | |
| hT4VL.FRv40510 | N27dE, R27fS, Q89Y, | 1.20 | 1.51 | 1.70 | 17.72 | 92 |
| | H94Q, M95P | | | | | |
| hT4VL.FRv40511 | N27dE, R27fS, Y91E, | 1.32 | 0.98 | 1.14 | 13.05 | 93 |
| | H94Q, M95P | | | | | |
| hT4VL.FRv40512 | N27dE, R27fS, Y91F, | 1.13 | 1.32 | 1.21 | 8.43 | 94 |
| | H94Q, M95P | | | | | |
| hT4VL.FRv40513 | N27dE, R27fV, Q89H, | 0.76 | 0.94 | 0.63 | 3.49 | 95 |
| | H94E, M95P | | | | | |
| hT4VL.FRv40514 | N27dE, R27fV, Q89Y, | 0.82 | 1.12 | 1.23 | 12.97 | 96 |
| | H94E, M95P | | | | | |
| hT4VL.FRv40515 | N27dE, R27fV, Y91E, | 1.09 | 0.86 | 0.85 | 11.88 | 97 |
| | H94E, M95P | | | | | |
| hT4VL.FRv40516 | N27dE, R27fV, Y91F, | 0.93 | 1.12 | 0.86 | 6.99 | 98 |
| | H94E, M95P | | | | | |
| hT4VL.FRv40517 | N27dE, R29S, Q89H, | 0.66 | 1.08 | 0.85 | 3.34 | 99 |
| | H94E, M95P | | | | | |
| hT4VL.FRv40518 | N27dE, R29S, Q89Y, | 0.94 | 1.13 | 1.32 | 8.30 | 100 |
| | H94E, M95P | | | | | |
| hT4VL.FRv40519 | N27dE, K30L, Q89H, | 0.91 | 1.11 | 1.51 | 4.80 | 101 |
| | H94E, M95P | | | | | |
| hT4VL.FRv40520 | N27dE, K30L, Q89Y, | 0.81 | 1.18 | 2.36 | 7.03 | 102 |
| | H94E, M95P | | | | | |
| hT4VL.FRv40521 | S27eD, R27fK, Y91E, | 1.01 | 1.11 | 1.15 | 17.89 | 103 |
| | H94E, M95P | | | | | |
| hT4VL.FRv40522 | S27eD, R27fK, Y91F, | 0.89 | 1.01 | 1.95 | 17.00 | 104 |
| | H94E, M95P | | | | | |
| hT4VL.FRv40523 | S27eD, R27fK, Y91E, | 1.28 | 1.28 | 2.57 | 30.18 | 105 |
| | H94Q, M95P | | | | | |
| hT4VL.FRv40524 | S27eD, R27fK, Y91F, | 1.16 | 1.44 | 3.20 | 26.77 | 106 |
| | H94Q, M95P | | | | | |
| hT4VL.FRv40525 | S27eD, R27fK, Y91E, | 1.22 | 1.41 | 2.41 | 24.81 | 107 |
| | H94Q | | | | | |
| hT4VL.FRv40526 | S27eD, R27fK, Y91F, | 0.98 | 1.45 | 2.83 | 15.70 | 108 |
| | H94Q | | | | | |
| hT4VL.FRv40527 | S27eE, R27fK, Y91E, | 1.03 | 0.98 | 1.51 | 18.54 | 109 |
| | H94E, M95P | | | | | |
| hT4VL.FRv40528 | S27eE, R27fK, Y91F, | 1.21 | 1.25 | 2.11 | 18.23 | 110 |
| | H94E, M95P | | | | | |
| hT4VL.FRv40529 | S27eE, R27fK, Y91E, | 1.19 | 1.08 | 2.35 | 26.72 | 111 |
| | H94Q, M95P | | | | | |
| hT4VL.FRv40530 | S27eE, R27fK, Y91F, | 1.13 | 1.43 | 3.49 | 28.07 | 112 |
| | H94Q, M95P | | | | | |
| hT4VL.FRv40531 | R27fS, R29S, Q89H, | 1.09 | 0.59 | 0.43 | 1.41 | 113 |
| | H94E, M95P | | | | | |
| hT4VL.FRv40532 | R27fS, R29S, Q89Y, | 0.99 | 0.64 | 0.58 | 6.44 | 114 |
| | H94E, M95P | | | | | |
| hT4VL.FRv40533 | R27fS, R29S, Y91E, | 1.06 | 0.62 | 0.44 | 2.36 | 115 |
| | H94E, M95P | | | | | |
| hT4VL.FRv40534 | R27fS, R29S, Y91F, | 0.97 | 0.79 | 0.48 | 1.80 | 116 |
| | H94E, M95P | | | | | |
| hT4VL.FRv40535 | R27fS, R29S, Q89H, | 1.16 | 0.84 | 0.49 | 2.14 | 117 |
| | H94Q, M95P | | | | | |
| hT4VL.FRv40536 | R27fS, R29S, Q89Y, | 1.10 | 0.82 | 0.70 | 15.71 | 118 |
| | H94Q, M95P | | | | | |
| hT4VL.FRv40537 | R27fS, R29S, Y91E, | 1.00 | 0.63 | 0.51 | 2.87 | 119 |
| | H94Q, M95P | | | | | |
| hT4VL.FRv40538 | R27fS, R29S, Y91F, | 1.33 | 0.56 | 0.72 | 3.84 | 120 |
| | H94Q, M95P | | | | | |
| hT4VL.FRv40539 | N27dE, R27fS, R29S, | 1.10 | 1.00 | 1.27 | 10.29 | 121 |
| | Q89Y, H94Q, M95P | | | | | |
| hT4VL.FRv40540 | N27dE, R27fS, R29S, | 1.25 | 0.60 | 1.03 | 4.09 | 122 |
| | Y91F, H94Q, M95P | | | | | |
| hT4VL.FRv4 | parent | 0.68 | 1.00 | 11.96 | 91.86 | 4 |
| hT4VL | hT4VL (original) | 0.40 | 1.22 | 14.22 | 86.07 | 174 |

### Example 3: Variants with FR1 Mutations

In the previous reports, hT4VL is a humanized VL which has two non-human germline residues in FR1 of human VK1 (Leucine at position 2 and Valine at position 3 corresponding to FR1 of mouse VK1) wherein mouse residue Leucine at position 2 was reported to be required for cytosol trafficking activity (Dong-Ki Choi et al., mAbs 6:6, 1402-1414; November/December 2014; WO2016013870). In the Example 1 and Example 2 of the present application, the same modified human VK1 FR1 was used during the FR re-selection.

28 variants identified in Example 2 were selected to additionally introduce substitution of Leucine to Isoleucine at position 2 (L2I) and substitution of Valine to Glutamine at position 3 (V3Q) into FR1 (i.e. back mutation of mouse residues to corresponding human residues of human VK1 FR1) such that the variants have a wild type human germline VK1 FR1 sequence (Table 7). We envisioned that such antibody variants with fully human VK1 FR1 are closer to human germline, and consequently have lower immunogenicity risk. Ab expression level, cytosol trafficking ability, ECM binding, and also SEC (size exclusion chromatography) profiles were assessed for the variants shown in Table 7.

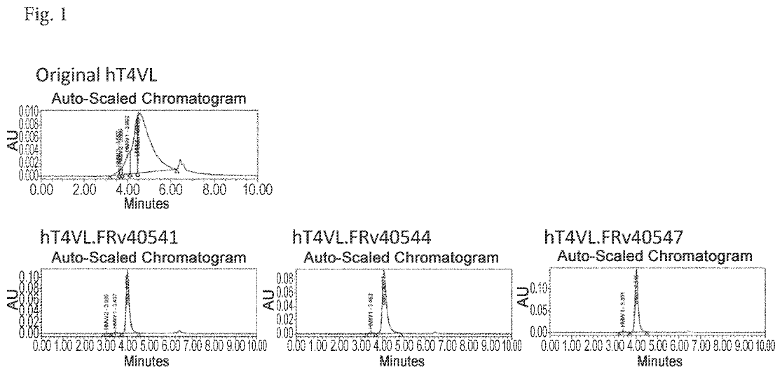

Aggregation profile was evaluated by Size-exclusion chromatography (SEC) analysis using UPLC (Acquity H-Class bio, Waters) for each variant. 50 mM Phosphate/300 mM NaCl (pH 7.0) was used for running buffer and BEH200 column (Waters, #186005225) was used as analytical column. Chromatogram was recorded by 220 nM UV absorption. Data was processed using Empower3 Software (Waters), and monomer peak percentage and full width at half maximum of the monomer peak were calculated. High monomer peak percentage and sharp symmetric peak shape (narrow peak width) are considered as good physicochemical property. The SEC analysis results were summarized in Table 7 (The variants which showed better profiles compared to the original hT4VL were shown in bold). While monomer peak percentage of original hT4VL was below 70% and peak width could not be calculated due to its asymmetric peak shape, all of the mutated variants listed in Table 7 showed better monomer peak percentage and sharper peak shape as shown in FIG. 1.

Notably, in contrast to the case of hT4VL which requires non-human germline residues of VK1 FR1 to retain its cytosol trafficking ability, variants with L2I/V3Q mutation (corresponding to a wild type fully human VK1 FR1 sequence) surprisingly exhibit improved or strong cytosol trafficking ability (Table 7), and improved Ab expression level, lower ECM binding, and better SEC (size exclusion chromatography) profiles.

Based on all the results, hT4VL.FRv40563 (L2I/V3Q/S27eD/R27fK/Y91F/H94Q, SEQ ID NO: 145) was selected as a template antibody for further CDRL2 optimization. As a result of comprehensive mutagenesis on the CDRL2 region, several mutations (such as W50A/G/I/T/V, S52F/I, T53N/Y, R54K/V, and those combinations) significantly improved Split Nluc signal compared to the template antibody while retaining low ECM binding (Table 8).

TABLE 7
Selected 28 variants with FR1 mutations (L2I/V3Q)

| | | | Split | ECM | ECM | | | | |
|---|---|---|---|---|---|---|---|---|---|
| | | | Nluc | binding | binding | | | | |
| | | Ab | (Fold | vs | vs | SEC | | | |
| | | expres- | change | Control | Control | mono- | SEC | SEC | |
| | | sion | vs | Ab | Ab | mer | peak | Reten- | SEQ |
| | | level | original | (pH | (pH | peak | width | tion | ID |
| Domain name | mutations | (mg/mL) | hT4VL) | 7.4) | 5.8) | % | @50% | time | NO |
| hT4VL.FRv40541 | L2I/V3Q/N27dE/R27fK/Q89H/H94E/M95P | 1.20 | 1.33 | 1.81 | 20.5 | 98.4 | 0.15 | 4.00 | 123 |
| hT4VL.FRv40542 | L2I/V3Q/N27dE/R27fK/Q89Y/H94E/M95P | 1.22 | 1.08 | 2.64 | 38.5 | 98.8 | 0.16 | 4.03 | 124 |
| hT4VL.FRv40543 | L2I/V3Q/N27dE/R27fK/Y91E/H94E/M95P | 1.35 | 1.13 | 2.49 | 32.0 | 98.6 | 0.20 | 4.08 | 125 |
| hT4VL.FRv40544 | L2I/V3Q/N27dE/R27fK/Y91F/H94E/M95P | 1.26 | 1.32 | 2.86 | 33.0 | 98.5 | 0.19 | 4.07 | 126 |
| hT4VL.FRv40545 | L2I/V3Q/N27dE/R27fS/Q89H/H94E/M95P | 1.42 | 1.14 | 1.28 | 8.6 | 97.4 | 0.14 | 4.00 | 127 |
| hT4VL.FRv40546 | L2I/V3Q/N27dE/R27fS/Q89Y/H94E/M95P | 1.55 | 1.00 | 1.54 | 22.3 | 98.2 | 0.14 | 4.02 | 128 |
| hT4VL.FRv40547 | L2I/V3Q/N27dE/R27fS/Y91F/H94E/M95P | 1.59 | 1.04 | 1.51 | 21.1 | 98.1 | 0.14 | 4.02 | 129 |
| hT4VL.FRv40548 | L2I/V3Q/N27dE/R27fS/Q89H/H94Q/M95P | 1.52 | 1.21 | 1.92 | 12.2 | 97.8 | 0.14 | 4.02 | 130 |
| hT4VL.FRv40549 | L2I/V3Q/N27dE/R27fS/Q89Y/H94Q/M95P | 1.24 | 1.14 | 2.58 | 27.8 | 98.3 | 0.14 | 4.05 | 131 |
| hT4VL.FRv40550 | L2I/V3Q/N27dE/R27fS/Y91E/H94Q/M95P | 1.88 | 1.23 | 2.12 | 27.7 | 98.6 | 0.15 | 4.05 | 132 |
| hT4VL.FRv40551 | L2I/V3Q/N27dE/R27fS/Y91F/H94Q/M95P | 1.63 | 1.25 | 2.19 | 24.1 | 98.3 | 0.15 | 4.03 | 133 |
| hT4VL.FRv40552 | L2I/V3Q/N27dE/R27fV/Q89Y/H94E/M95P | 1.55 | 0.91 | 2.02 | 24.0 | 98.2 | 0.14 | 4.05 | 134 |
| hT4VL.FRv40553 | L2I/V3Q/N27dE/R27fV/Y91F/H94E/M95P | 1.70 | 1.04 | 1.84 | 25.8 | 98.1 | 0.15 | 4.07 | 135 |
| hT4VL.FRv40554 | L2I/V3Q/N27dE/R29S/Q89H/H94E/M95P | 1.34 | 0.85 | 1.15 | 7.2 | 97.7 | 0.14 | 3.98 | 136 |
| hT4VL.FRv40555 | L2I/V3Q/N27dE/R29S/Q89Y/H94E/M95P | 1.63 | 1.08 | 1.98 | 19.5 | 98.3 | 0.14 | 4.0 | 137 |
| hT4VL.FRv40556 | L2I/V3Q/N27dE/K30L/Q89H/H94E/M95P | 1.23 | 0.90 | 2.49 | 8.3 | 96.8 | 0.14 | 4.00 | 138 |
| hT4VL.FRv40557 | L2I/V3Q/N27dE/K30L/Q89Y/H94E/M95P | 1.28 | 1.09 | 2.65 | 9.6 | 98.2 | 0.14 | 4.04 | 139 |
| hT4VL.FRv40558 | L2I/V3Q/S27eD/R27IK/Y91E/H94E/M95P | 1.57 | 1.28 | 2.31 | 46.2 | 98.7 | 0.21 | 4.07 | 140 |
| hT4VL.FRv40559 | L2I/V3Q/S27eD/R27fK/Y91F/H94E/M95P | 1.28 | 1.30 | 2.40 | 41.9 | 98.7 | 0.21 | 4.08 | 141 |
| hT4VL.FRv40560 | L2I/V3Q/S27eD/R27fK/Y91E/H94Q/M95P | 1.29 | 1.12 | 3.13 | 56.7 | 98.8 | 0.25 | 4.14 | 142 |
| hT4VL.FRv40561 | L2I/V3Q/S27eD/R27fK/Y91F/H94Q/M95P | 1.41 | 1.37 | 3.83 | 59.1 | 98.9 | 0.25 | 4.14 | 143 |
| hT4VL.FRv40562 | L2I/V3Q/S27eD/R27fK/Y91E/H94Q | 1.40 | 1.23 | 2.03 | 31.1 | 98.8 | 0.23 | 4.10 | 144 |
| hT4VL.FRv40563 | L2I/V3Q/S27eD/R27fK/Y91F/H94Q | 1.62 | 1.38 | 3.01 | 25.1 | 98.8 | 0.21 | 4.08 | 145 |
| hT4VL.FRv40564 | L2I/V3Q/S27eE/R27fK/Y91E/H94E/M95P | 1.90 | 1.18 | 2.65 | 42.5 | 98.6 | 0.21 | 4.07 | 146 |
| hT4VL.FRv40565 | L2I/V3Q/S27eE/R27fK/Y91F/H94E/M95P | 1.75 | 1.43 | 2.62 | 39.7 | 98.6 | 0.22 | 4.09 | 147 |
| hT4VL.FRv40566 | L2I/V3Q/S27eE/R27fK/Y91E/H94Q/M95P | 1.69 | 1.29 | 3.24 | 50.0 | 98.8 | 0.27 | 4.15 | 148 |
| hT4VL.FRv40567 | L2I/V3Q/S27eE/R27fK/Y91F/H94Q/M95P | 1.55 | 1.30 | 3.81 | 49.2 | 98.9 | 0.26 | 4.15 | 149 |
| hT4VL.FRv40568 | L2I/V3Q/N27dE/R27fS/R29S/Q89Y/H94Q/M95P | 1.18 | 0.94 | 1.76 | 11.8 | 97.4 | 0.14 | 4.01 | 150 |
| hT4VL.FRv4 | parent | 1.15 | 0.89 | 9.89 | 79.7 | 90.8 | 0.28 | 4.04 | 1 |
| hT4VL | original | 0.57 | 1.00 | 11.29 | 72.7 | 66.9 | | 4.50 | 174 |

TABLE 8
Variants with further mutations in CDRL2 region of hT4VL.FRv40563

| | | | Split | ECM | ECM | | | |
|---|---|---|---|---|---|---|---|---|
| | | | Nluc | binding | binding | | | |
| | | Ab | (Fold | vs | vs | SEC | | |
| | | expres- | change | Control | Control | mono- | SEC | |
| | | sion | vs | Ab | Ab | mer | peak | SEQ |
| | | level | original | (pH | (pH | peak | width | ID |
| Domain name | Mutations | (mg/mL) | hT4VL) | 7.4) | 5.8) | % | @50% | NO |
| hT4VL.FRv40569 | L2I, V3Q, S27eD, R27fK, W50A, Y91F, H94Q | 1.07 | 1.48 | 2.70 | 23.0 | 92.37 | 0.182807 | 161 |
| hT4VL.FRv40573 | L2I, V3Q, S27eD, R27fK, W50G, Y91F, H94Q | 1.56 | 1.27 | 3.13 | 20.9 | 99.19 | 0.17619 | 162 |
| hT4VL.FRv40575 | L2I, V3Q, S27cD, R27fK, W50I, Y91F, H94Q | 1.39 | 1.41 | 3.83 | 19.0 | 99.48 | 0.188645 | 163 |
| hT4VL.FRv40584 | L2I, V3Q, S27eD, R27fK, W50T, Y91F, H94Q | 1.53 | 1.34 | 3.46 | 24.1 | 99.12 | 0.1945 | 164 |
| hT4VL.FRv40585 | L2I, V3Q, S27eD, R27fK, W50V, Y91F, H94Q | 1.10 | 1.60 | 3.64 | 21.4 | 99.39 | 0.276359 | 165 |
| hT4VL.FRv40608 | L2I, V3Q, S27eD, R27fK, S52F, Y91F, H94Q | 0.91 | 1.04 | 3.91 | 17.8 | 99.22 | 0.185157 | 166 |
| hT4VL.FRv40611 | L2I, V3Q, S27eD, R27fK, S52I, Y91F, H94Q | 1.22 | 1.14 | 3.66 | 19.3 | 98.92 | 0.200182 | 167 |
| hT4VL.FRv40633 | L2I, V3Q, S27eD, R27fK, T53N, Y91F, H94Q | 1.73 | 1.40 | 4.08 | 18.7 | 98.69 | 0.279574 | 168 |
| hT4VL.FRv40640 | L2I, V3Q, S27eD, R27fK, T53Y, Y91F, H94Q | 1.44 | 1.19 | 5.01 | 22.4 | 98.61 | 0.295898 | 169 |
| hT4VL.FRv40648 | L2I, V3Q, S27eD, R27fK, R54K, Y91F, H94Q | 1.41 | 1.15 | 3.12 | 13.4 | 98.88 | 0.19084 | 170 |
| hT4VL.FRv40696 | L2I, V3Q, S27eD, R27fK, W50I, S52F, Y91F, H94Q | 0.97 | 1.15 | 3.98 | 18.0 | 99.92 | 0.30921 | 171 |
| hT4VL.FRv40697 | L2I, V3Q, S27eD, R27fK, W50I, T53N, Y91F, H94Q | 1.21 | 1.32 | 4.40 | 16.8 | 93.43 | 0.682695 | 172 |
| hT4VL.FRv40698 | L2I, V3Q, S27eD, R27fK, W50I, R54V, Y91F, H94Q | 1.22 | 1.20 | 2.23 | 8.6 | 99.34 | 0.145848 | 173 |
| hT4VL.FRv40563 | L2I, V3Q, S27eD, R27fK, Y91F, H94Q (Template) | 1.45 | 1.05 | 3.64 | 16.0 | 98.93 | 0.271813 | 145 |
| hT4VL | Original | 1.55 | 1.00 | 13.71 | 69.2 | 68.08 | N.A. | 174 |

### Example 4: Preparation of Bifunctional Antibodies Comprising a Cytosol-Penetrating Domain and a Cell Surface Antigen-Binding Domain; and Imaging Analysis of Cytosol Trafficking of Bifunctional Antibodies in ASGPR Expressing Cell Lines

Attempts were made to construct bifunctional and multivalent antibodies from a cytosol-penetrating antibody identified in Example 3 and an antibody against a cell surface antigen, in order to specifically deliver the bifunctional antibodies to the cytosol of target cells in a cell surface antigen-dependent manner as reported in WO2020022262.

Imaging analysis was conducted to evaluate cytosol penetrating ability of bifunctional antibodies in a receptor-dependent manner. Antibodies were transiently expressed using Expi293 cell line (Thermo Fisher, Carlsbad, CA, USA) and purified by methods known to those skilled in the art. Antibodies listed in Table 9 were used to prepare bifunctional antibodies listed in Table 10. Bifunctional antibodies were prepared by methods know to those skilled in the art. To target ASGPR, anti-ASGPR antibody AGA0078 (VH: SEQ ID NO: 159, VL: SEQ ID NO: 156) was used.

In the imaging analysis, ASGPR expressing HeLa cell line (HeLa/ASGPR/BirA) described in Reference Example 3 was used. HeLa/ASGPR/BirA cells were cultured in Minimum Essential Medium Eagle (Sigma, Cat #M4655-500ML) containing 10% FBS (Sigma Aldrich, Cat #172012-500ML), Penicillin-Streptomycin (Gibco, Cat #15140-122), 750 micro g/mL Geneticin (Thermo Fisher Scientific, Cat #10131-027) and 100 micro g/mL of zeocin (Thermo Fisher Scientific, Cat #R25001). Suspension of HeLa/ASGPR/BirA was seeded at 50 micro L/well (6×10³ cells/well) in BioCoat Collagen I Multiwell Plates 96-well Black/clear (Corning, Cat #354649). Then, cells were cultured overnight in a humidified 5% CO₂, 37 degrees C. incubator. After removing the culture supernatant, cells were incubated with 3 micro M of antibodies in 50 micro L/well of culture medium for 3 hours in a humidified 5% CO₂, 37 degrees C. incubator. After incubation, cells were washed twice in 200 mM Glycine (Wako Pure Chemical Industries, Cat #077-00735)—HCl (Wako Pure Chemical Industries, Cat #083-01095), 150 mM NaCl (Wako Pure Chemical Industries, Cat #191-01665), pH 2.5. Then, cells were fixed by 4% paraformaldehyde-phosphate buffer (Nacalai Tesque, Cat #09154-56) at room temperature for 15 minutes and permeabilized by PBS containing 0.5% Triton X-100 (Bio-Rad, Cat #161-0407) and 5% FBS. The solution used in the permeabilization was also used in the subsequent steps for incubating with the secondary antibody and washing. Cells were incubated with 1/500-diluted Goat Anti-Human IgG-AF488 (SouthernBiotech, Cat #2040-30) and 1/2000-diluted Hoechst 33342 (Thermo Fisher Scientific, Cat #H3570) for 30 min at room temperature. After washing twice, cells were subjected to imaging analysis using Operetta CLS High-Content Analysis System (Perkin Elmer). Fluorescence intensity of Alexa488 was evaluated by using Harmony software (Perkin Elmer).

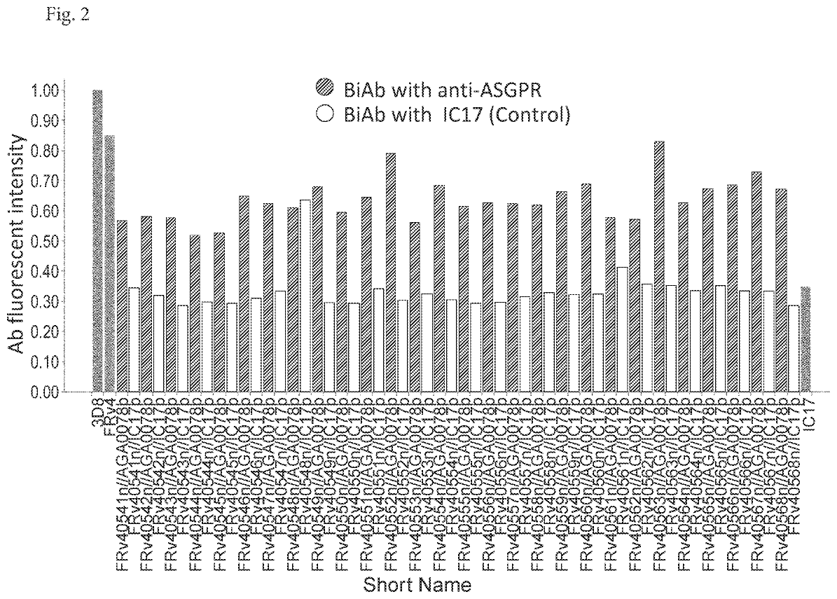

As a result, antibodies selected in Example 3 in bifunctional molecular format with anti-ASGPR antibody (AGA0078) showed higher Alexa488 fluorescence intensity than in bifunctional molecular format with anti-KLH antibody (IC17) (FIG. 2). This indicates that bifunctional antibody could be delivered into HeLa/ASGPR/BirA cells by ASGPR dependent manner, supporting receptor dependent cytosol trafficking observed by Split-NanoLuc assay in Example 3.

TABLE 9
List of parent antibodies used for preparing bifunctional antibodies and control antibodies

| | | | VH | | CH | | VL | | CL |
|---|---|---|---|---|---|---|---|---|---|
| Short | | | SEQ | | SEQ | | SEQ | | SEQ |
| Name | Sample name | VH | ID | CH | ID | VL | ID | CL | ID |
| FRv40541n | 3D8VH-SG1.S3n/hT4VL.FRv40541-KT0 | 3D8VH | 158 | SG1.S3n | 154 | hT4VL.FRv40541 | 123 | KT0 | 12 |
| 40542-KTn | 3D8VH-SG1.S3n/hT4VL.FRv40542-KT0 | 3D8VH | 158 | SG1.S3n | 154 | hT4VL.FRv40542 | 124 | KT0 | 12 |
| 40543-KTn | 3D8VH-SG1.S3n/hT4VL.FRv40543-KT0 | 3D8VH | 158 | SG1.S3n | 154 | hT4VL.FRv40543 | 125 | KT0 | 12 |
| 40544-KTn | 3D8VH-SG1.S3n/hT4VL.FRv40544-KT0 | 3D8VH | 158 | SG1.S3n | 154 | hT4VL.FRv40544 | 126 | KT0 | 12 |
| 40545-KTn | 3D8VH-SG1.S3n/hT4VL.FRv40545-KT0 | 3D8VH | 158 | SG1.S3n | 154 | hT4VL.FRv40545 | 127 | KT0 | 12 |
| 40546-KTn | 3D8VH-SG1.S3n/hT4VL.FRv40546-KT0 | 3D8VH | 158 | SG1.S3n | 154 | hT4VL.FRv40546 | 128 | KT0 | 12 |
| 40547-KTn | 3D8VH-SG1.S3n/hT4VL.FRv40547-KT0 | 3D8VH | 158 | SG1.S3n | 154 | hT4VL.FRv40547 | 129 | KT0 | 12 |
| 40548-KTn | 3D8VH-SG1.S3n/hT4VL.FRv40548-KT0 | 3D8VH | 158 | SG1.S3n | 154 | hT4VL.FRv40548 | 130 | KT0 | 12 |
| 40549-KTn | 3D8VH-SG1.S3n/hT4VL.FRv40549-KT0 | 3D8VH | 158 | SG1.S3n | 154 | hT4VL.FRv40549 | 131 | KT0 | 12 |
| 40550-KTn | 3D8VH-SG1.S3n/hT4VL.FRv40550-KT0 | 3D8VH | 158 | SG1.S3n | 154 | hT4VL.FRv40550 | 132 | KT0 | 12 |
| FRv40551n | 3D8VH-SG1.S3n/hT4VL.FRv40551-KT0 | 3D8VH | 158 | SG1.S3n | 154 | hT4VL.FRv40551 | 133 | KT0 | 12 |
| FRv40552n | 3D8VH-SG1.S3n/hT4VL.FRv40552-KT0 | 3D8VH | 158 | SG1.S3n | 154 | hT4VL.FRv40552 | 134 | KT0 | 12 |
| 40553-KTn | 3D8VH-SG1.S3n/hT4VL.FRv40553-KT0 | 3D8VH | 158 | SG1.S3n | 154 | hT4VL.FRv40553 | 135 | KT0 | 12 |
| 40554-KTn | 3D8VH-SG1.S3n/hT4VL.FRv40554-KT0 | 3D8VH | 158 | SG1.S3n | 154 | hT4VL.FRv40554 | 136 | KT0 | 12 |
| 40555-KTn | 3D8VH-SG1.S3n/hT4VL.FRv40555-KT0 | 3D8VH | 158 | SG1.S3n | 154 | hT4VL.FRv40555 | 137 | KT0 | 12 |
| 40556-KTn | 3D8VH-SG1.S3n/hT4VL.FRv40556-KT0 | 3D8VH | 158 | SG1.S3n | 154 | hT4VL.FRv40556 | 138 | KT0 | 12 |
| 40557-KTn | 3D8VH-SG1.S3n/hT4VL.FRv40557-KT0 | 3D8VH | 158 | SG1.S3n | 154 | hT4VL.FRv40557 | 139 | KT0 | 12 |
| FRv40558n | 3D8VH-SG1.S3n/hT4VL.FRv40558-KT0 | 3D8VH | 158 | SG1.S3n | 154 | hT4VL.FRv40558 | 140 | KT0 | 12 |
| FRv40559n | 3D8VH-SG1.S3n/hT4VL.FRv40559-KT0 | 3D8VH | 158 | SG1.S3n | 154 | hT4VL.FRv40559 | 141 | KT0 | 12 |
| FRv40560n | 3D8VH-SG1.S3n/hT4VL.FRv40560-KT0 | 3D8VH | 158 | SG1.S3n | 154 | hT4VL.FRv40560 | 142 | KT0 | 12 |
| 40561-KTn | 3D8VH-SG1.S3n/hT4VL.FRv40561-KT0 | 3D8VH | 158 | SG1.S3n | 154 | hT4VL.FRv40561 | 143 | KT0 | 12 |
| 40562-KTn | 3D8VH-SG1.S3n/hT4VL.FRv40562-KT0 | 3D8VH | 158 | SG1.S3n | 154 | hT4VL.FRv40562 | 144 | KT0 | 12 |
| FRv40563n | 3D8VH-SG1.S3n/hT4VL.FRv40563-KT0 | 3D8VH | 158 | SG1.S3n | 154 | hT4VL.FRv40563 | 145 | KT0 | 12 |
| FRv40564n | 3D8VH-SG1.S3n/hT4VL.FRv40564-KT0 | 3D8VH | 158 | SG1.S3n | 154 | hT4VL.FRv40564 | 146 | KT0 | 12 |
| 40565-KTn | 3D8VH-SG1.S3n/hT4VL.FRv40565-KT0 | 3D8VH | 158 | SG1.S3n | 154 | hT4VL.FRv40565 | 147 | KT0 | 12 |
| 40566-KTn | 3D8VH-SG1.S3n/hT4VL.FRv40566-KT0 | 3D8VH | 158 | SG1.S3n | 154 | hT4VL.FRv40566 | 148 | KT0 | 12 |
| FRv40567n | 3D8VH-SG1.S3n/hT4VL.FRv40567-KT0 | 3D8VH | 158 | SG1.S3n | 154 | hT4VL.FRv40567 | 149 | KT0 | 12 |
| FRv40568n | 3D8VH-SG1.S3n/hT4VL.FRv40568-KT0 | 3D8VH | 158 | SG1.S3n | 154 | hT4VL.FRv40568 | 150 | KT0 | 12 |
| 3D8n | 3D8VH-SG1.S3n/hT4VL-KT0 | 3D8VH | 158 | SG1.S3n | 154 | hT4VL | 174 | KT0 | 12 |
| FRv4n | 3D8VH-SG1.S3n/hT4VL.FRv4-KT0 | 3D8VH | 158 | SG1.S3n | 154 | hT4VL.FRv4 | 4 | KT0 | 12 |
| AGA0078p | AGA0078Ha-SG1.S3p/AGA0078La-KT0 | AGA0078Ha | 159 | SG1.S3p | 155 | AGA0078La | 156 | KT0 | 12 |
| IC17p | IC17HdK-SG1.S3p/IC17L-KT0 | IC17HdK | 160 | SG1.S3p | 155 | IC17L | 157 | KT0 | 12 |

TABLE 10
List of bifunctional antibodies and control antibodies used in imaging analysis

| Bifunctional antibody | First arm | Second arm |
|---|---|---|
| FRv40542n//AGA0078p | 3D8VH- | AGA0078Ha- |
| | SG1.S3n/hT4VL.FRv40542- | SG1.S3p/AGA0078La-KT0 |
| | KT0 | |
| FRv40543n//AGA0078p | 3D8VH- | AGA0078Ha- |
| | SG1.S3n/hT4VL.FRv40543- | SG1.S3p/AGA0078La-KT0 |
| | KT0 | |
| FRv40544n//AGA0078p | 3D8VH- | AGA0078Ha- |
| | SG1.S3n/hT4VL.FRv40544- | SG1.S3p/AGA0078La-KT0 |
| | KT0 | |
| FRv40545n//AGA0078p | 3D8VH- | AGA0078Ha- |
| | SG1.S3n/hT4VL.FRv40545- | SG1.S3p/AGA0078La-KT0 |
| | KT0 | |
| FRv40546n//AGA0078p | 3D8VH- | AGA0078Ha- |
| | SG1.S3n/hT4VL.FRv40546- | SG1.S3p/AGA0078La-KT0 |
| | KT0 | |
| FRv40547n//AGA0078p | 3D8VH- | AGA0078Ha- |
| | SG1.S3n/hT4VL.FRv40547- | SG1.S3p/AGA0078La-KT0 |
| | KT0 | |
| FRv40548n//AGA0078p | 3D8VH- | AGA0078Ha- |
| | SG1.S3n/hT4VL.FRv40548- | SG1.S3p/AGA0078La-KT0 |
| | KT0 | |
| FRv40549n//AGA0078p | 3D8VH- | AGA0078Ha- |
| | SG1.S3n/hT4VL.FRv40549- | SG1.S3p/AGA0078La-KT0 |
| | KT0 | |
| FRv40550n//AGA0078p | 3D8VH- | AGA0078Ha- |
| | SG1.S3n/hT4VL.FRv40550- | SG1.S3p/AGA0078La-KT0 |
| | KT0 | |
| FRv40553n//AGA0078p | 3D8VH- | AGA0078Ha- |
| | SG1.S3n/hT4VL.FRv40553- | SG1.S3p/AGA0078La-KT0 |
| | KT0 | |
| FRv40554n//AGA0078p | 3D8VH- | AGA0078Ha- |
| | SG1.S3n/hT4VL.FRv40554- | SG1.S3p/AGA0078La-KT0 |
| | KT0 | |
| FRv40555n//AGA0078p | 3D8VH- | AGA0078Ha- |
| | SG1.S3n/hT4VL.FRv40555- | SG1.S3p/AGA0078La-KT0 |
| | KT0 | |
| FRv40556n//AGA0078p | 3D8VH- | AGA0078Ha- |
| | SG1.S3n/hT4VL.FRv40556- | SG1.S3p/AGA0078La-KT0 |
| | KT0 | |
| FRv40557n//AGA0078p | 3D8VH- | AGA0078Ha- |
| | SG1.S3n/hT4VL.FRv40557- | SG1.S3p/AGA0078La-KT0 |
| | KT0 | |
| FRv40561n//AGA0078p | 3D8VH- | AGA0078Ha- |
| | SG1.S3n/hT4VL.FRv40561- | SG1.S3p/AGA0078La-KT0 |
| | KT0 | |
| FRv40562n//AGA0078p | 3D8VH- | AGA0078Ha- |
| | SG1.S3n/hT4VL.FRv40562- | SG1.S3p/AGA0078La-KT0 |
| | KT0 | |
| FRv40565n//AGA0078p | 3D8VH- | AGA0078Ha- |
| | SG1.S3n/hT4VL.FRv40565- | SG1.S3p/AGA0078La-KT0 |
| | KT0 | |
| FRv40566n//AGA0078p | 3D8VH- | AGA0078Ha- |
| | SG1.S3n/hT4VL.FRv40566- | SG1.S3p/AGA0078La-KT0 |
| | KT0 | |
| FRv40542n//IC17p | 3D8VH- | IC17HdK-SG1.S3p/IC17L-KT0 |
| | SG1.S3n/hT4VL.FRv40542- | |
| | KT0 | |
| FRv40543n//IC17p | 3D8VH- | IC17HdK-SG1.S3p/IC17L-KT0 |
| | SG1.S3n/hT4VL.FRv40543- | |
| | KT0 | |
| FRv40544n//IC17p | 3D8VH- | IC17HdK-SG1.S3p/IC17L-KT0 |
| | SG1.S3n/hT4VL.FRv40544- | |
| | KT0 | |
| FRv40545n//IC17p | 3D8VH- | IC17HdK-SG1.S3p/IC17L-KT0 |
| | SG1.S3n/hT4VL.FRv40545- | |
| | KT0 | |
| FRv40546n//IC17p | 3D8VH- | IC17HdK-SG1.S3p/IC17L-KT0 |
| | SG1.S3n/hT4VL.FRv40546- | |
| | KT0 | |
| FRv40547n//IC17p | 3D8VH- | IC17HdK-SG1.S3p/IC17L-KT0 |
| | SG1.S3n/hT4VL.FRv40547- | |
| | KT0 | |
| FRv40548n//IC17p | 3D8VH- | IC17HdK-SG1.S3p/IC17L-KT0 |
| | SG1.S3n/hT4VL.FRv40548- | |
| | KT0 | |
| FRv40549n//IC17p | 3D8VH- | IC17HdK-SG1.S3p/IC17L-KT0 |
| | SG1.S3n/hT4VL.FRv40549- | |
| | KT0 | |
| FRv40550n//IC17p | 3D8VH- | IC17HdK-SG1.S3p/IC17L-KT0 |
| | SG1.S3n/hT4VL.FRv40550- | |
| | KT0 | |
| FRv40553n//IC17p | 3D8VH- | IC17HdK-SG1.S3p/IC17L-KT0 |
| | SG1.S3n/hT4VL.FRv40553- | |
| | KT0 | |
| FRv40554n//IC17p | 3D8VH- | IC17HdK-SG1.S3p/IC17L-KT0 |
| | SG1.S3n/hT4VL.FRv40554- | |
| | KT0 | |
| FRv40555n//IC17p | 3D8VH- | IC17HdK-SG1.S3p/IC17L-KT0 |
| | SG1.S3n/hT4VL.FRv40555- | |
| | KT0 | |
| FRv40556n//IC17p | 3D8VH- | IC17HdK-SG1.S3p/IC17L-KT0 |
| | SG1.S3n/hT4VL.FRv40556- | |
| | KT0 | |
| FRv40557n//IC17p | 3D8VH- | IC17HdK-SG1.S3p/IC17L-KT0 |
| | SG1.S3n/hT4VL.FRv40557- | |
| | KT0 | |
| FRv40561n//IC17p | 3D8VH- | IC17HdK-SG1.S3p/IC17L-KT0 |
| | SG1.S3n/hT4VL.FRv40561- | |
| | KT0 | |
| FRv40562n//IC17p | 3D8VH- | IC17HdK-SG1.S3p/IC17L-KT0 |
| | SG1.S3n/hT4VL.FRv40562- | |
| | KT0 | |
| FRv40565n//IC17p | 3D8VH- | IC17HdK-SG1.S3p/IC17L-KT0 |
| | SG1.S3n/hT4VL.FRv40565- | |
| | KT0 | |
| FRv40566n//IC17p | 3D8VH- | IC17HdK-SG1.S3p/IC17L-KT0 |
| | SG1.S3n/hT4VL.FRv40566- | |
| | KT0 | |
| FRv40541n//AGA0078p | 3D8VH- | AGA0078Ha- |
| | SG1.S3n/hT4VL.FRv40541- | SG1.S3p/AGA0078La-KT0 |
| | KT0 | |
| FRv40551n//AGA0078p | 3D8VH- | AGA0078Ha- |
| | SG1.S3n/hT4VL.FRv40551- | SG1.S3p/AGA0078La-KT0 |
| | KT0 | |
| FRv40552n//AGA0078p | 3D8VH- | AGA0078Ha- |
| | SG1.S3n/hT4VL.FRv40552- | SG1.S3p/AGA0078La-KT0 |
| | KT0 | |
| FRv40558n//AGA0078p | 3D8VH- | AGA0078Ha- |
| | SG1.S3n/hT4VL.FRv40558- | SG1.S3p/AGA0078La-KT0 |
| | KT0 | |
| FRv40559n//AGA0078p | 3D8VH- | AGA0078Ha- |
| | SG1.S3n/hT4VL.FRv40559- | SG1.S3p/AGA0078La-KT0 |
| | KT0 | |
| FRv40560n//AGA0078p | 3D8VH- | AGA0078Ha- |
| | SG1.S3n/hT4VL.FRv40560- | SG1.S3p/AGA0078La-KT0 |
| | KT0 | |
| FRv40563n//AGA0078p | 3D8VH- | AGA0078Ha- |
| | SG1.S3n/hT4VL.FRv40563- | SG1.S3p/AGA0078La-KT0 |
| | KT0 | |
| FRv40564n//AGA0078p | 3D8VH- | AGA0078Ha- |
| | SG1.S3n/hT4VL.FRv40564- | SG1.S3p/AGA0078La-KT0 |
| | KT0 | |
| FRv40567n//AGA0078p | 3D8VH- | AGA0078Ha- |
| | SG1.S3n/hT4VL.FRv40567- | SG1.S3p/AGA0078La-KT0 |
| | KT0 | |
| FRv40568n//AGA0078p | 3D8VH- | AGA0078Ha- |
| | SG1.S3n/hT4VL.FRv40568- | SG1.S3p/AGA0078La-KT0 |
| | KT0 | |
| FRv40541n//IC17p | 3D8VH- | IC17HdK-SG1.S3p/IC17L-KT0 |
| | SG1.S3n/hT4VL.FRv40541- | |
| | KT0 | |
| FRv40551n//IC17p | 3D8VH- | IC17HdK-SG1.S3p/IC17L-KT0 |
| | SG1.S3n/hT4VL.FRv40551- | |
| | KT0 | |
| FRv40552n//IC17p | 3D8VH- | IC17HdK-SG1.S3p/IC17L-KT0 |
| | SG1.S3n/hT4VL.FRv40552- | |
| | KT0 | |
| FRv40558n//IC17p | 3D8VH- | IC17HdK-SG1.S3p/IC17L-KT0 |
| | SG1.S3n/hT4VL.FRv40558- | |
| | KT0 | |
| FRv40559n//IC17p | 3D8VH- | IC17HdK-SG1.S3p/IC17L-KT0 |
| | SG1.S3n/hT4VL.FRv40559- | |
| | KT0 | |
| FRv40560n//IC17p | 3D8VH- | IC17HdK-SG1.S3p/IC17L-KT0 |
| | SG1.S3n/hT4VL.FRv40560- | |
| | KT0 | |
| FRv40563n//IC17p | 3D8VH- | IC17HdK-SG1.S3p/IC17L-KT0 |
| | SG1.S3n/hT4VL.FRv40563- | |
| | KT0 | |
| FRv40564n//IC17p | 3D8VH- | IC17HdK-SG1.S3p/IC17L-KT0 |
| | SG1.S3n/hT4VL.FRv40564- | |
| | KT0 | |
| FRv40567n//IC17p | 3D8VH- | IC17HdK-SG1.S3p/IC17L-KT0 |
| | SG1.S3n/hT4VL.FRv40567- | |
| | KT0 | |
| FRv40568n//IC17p | 3D8VH- | IC17HdK-SG1.S3p/IC17L-KT0 |
| | SG1.S3n/hT4VL.FRv40568- | |
| | KT0 | |
| 3D8 | 3D8VH-SG1.S3n/hT4VL-KT0 | — |
| FRv4 | 3D8VH- | — |
| | SG1.S3n/hT4VL.FRv4-KT0 | |
| IC17 | IC17HdK-SG1.S3p/IC17L- | — |
| | KT0 | |

### Example 5: Cytosol Trafficking Ability was Confirmed with Non-3D8 Heavy Chain

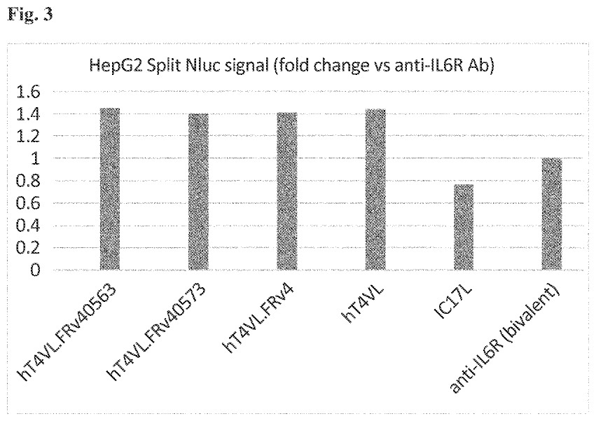

Although the identical heavy chain of 3D8 (SEQ ID NO: 13) was used for the above Examples 1-4, the present inventors further evaluated whether the newly identified light chain (VL) variants in Examples 1-4 still retain their cytosol trafficking ability when combined with other non-3D8 heavy chain. For this purpose, an anti-IL6R heavy chain, AL1W8P226 (SEQ ID NO: 151) was used as a control to combine with a VL variant identified in Examples 1-4. Bifunctional antibodies comprising the anti-IL6R heavy chain and a VL variant identified in Examples 1-4 were prepared, and tested for the cytosol trafficking ability by Split Nluc assay using HepG2 cells which intrinsically expresses IL6R on the cell surface. As a result, antibodies comprising the VL variant with AL1W8P226 showed higher Split Nluc signal compared to the bivalent anti-IL6R antibody (heavy chain SEQ ID NO: 152, light chain SEQ ID NO: 153) and also the antibody comprising AL1W8P226 with negative control light chain IC17L (SEQ ID NO: 154), suggesting that the VL variants exhibits cytosol trafficking activity and target cell (IL6R)-specific cytosol trafficking can be mediated by IL6R binding (FIG. 3).

### Example 6: Antibody Pharmacokinetics (PK) Profile of the Optimized Variants

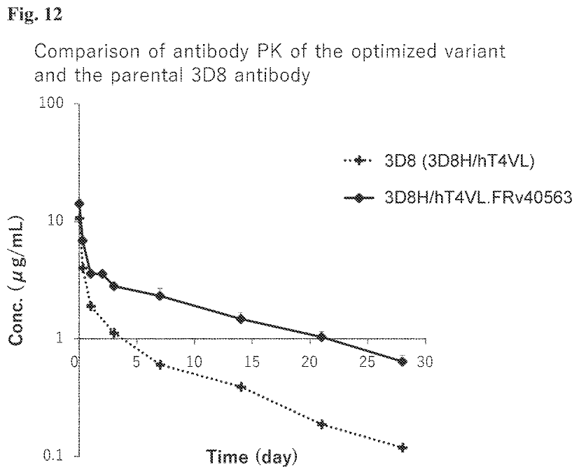

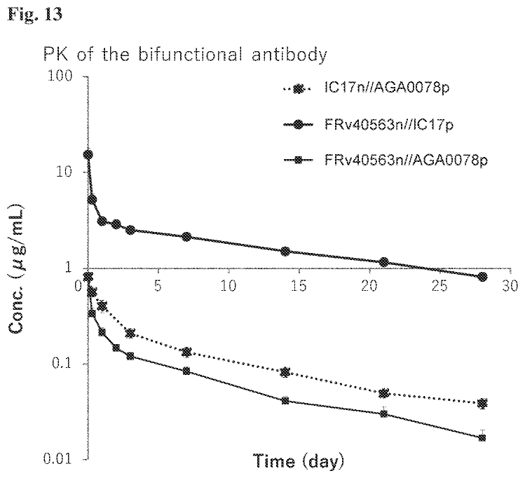

As demonstrated in Example 2 and Example 3, the present inventors successfully identified cell-penetrating antibodies with dramatically improved physicochemical properties such as ECM binding and SEC profile which could affect antibody PK. One of the selected variants 3D8H/hT4VL.FRv40563 (VH: SEQ ID NO:158, VL: SEQ ID NO: 145) was subjected to a mouse intravenous administration test. After antibody administration, blood was sampled over time and plasma was obtained. Then the concentration of the administered antibody in plasma was measured by ELISA method. Time-course data of the antibody concentration in plasma were analyzed with WinNonlin ver 7.0 (Certara) to calculate antibody clearance (CL). As a result, the antibody PK profile of the newly identified variant (3D8H/hT4VL.FRv40563) was remarkably improved compared to the 3D8 (3D8H/hT4VL) (FIG. 12). Furthermore, PK of the bifunctional antibody comprising a cytosol-penetrating domain (FRv40563) and a cell surface antigen-binding domain (anti-ASGPR antibody, AGA0078) was also assessed. ASGPR is mainly expressed in liver and bivalent anti-ASGPR antibody showed very fast elimination. As shown in FIG. 13, FRv40563n//AGA0078p showed faster clearance compared to its control antibody FRv40563n//IC17p, suggesting that the bifunctional antibody FRv40563n//AGA0078p could be delivered to liver by ASGPR dependent manner. In addition, FRv40563n//AGA0078p showed faster clearance than IC17n//AGA0078p. This could support ASGPR dependent cytosol trafficking in vivo.

### Reference Example 1: Split NanoLuc Assay to Assess Cytosol Trafficking Ability

### (1) Concept of Split NanoLuc Assay

NanoLuc is a small 19 kDa luciferase enzyme engineered from a deep sea luminous shrimp. The split NanoLuc assay utilizes NanoLuc which is cleaved into two sub-units—a small 1.3 kDa Small BiT (SmBiT) and a larger 18 kDa Large BiT (LgBiT). Complementation of the LgBiT and SmBiT is observed as luminescence from enzymatic reaction on substrate in live cells by use of the NanoLuc Live Cell Assay System (Promega). In the split NanoLuc assay, HeLa cells were genetically modified to stably express NanoLuc LgBiT intracellularly. The NanoLuc LgBiT was either expressed as free cytosolic protein, or tethered to an intracellular structure within the cytosol, for instance, the mitochondrial outer membrane. The tethering of the NanoLuc LgBiT to the mitochondrial outer membrane resulted in significantly lower background signal and better assay sensitivity compared to when NanoLuc LgBiT was expressed as free cytosolic protein. This was despite the tethered NanoLuc LgBiT having an overall lower expression as determined by GFP. The SmBiT was fused or conjugated with the antibodies tested. Penetration of antibody-SmBiT conjugates into the cytosol would complement the NanoLuc LgBiT to form a luciferase enzyme, which could be detected by enhancement of luminescence.

### (2) Cell Line Construction

To generate HeLa cells stably expressing human ASGPR, cells were transfected with plasmids encoding human ASGPR-H1 (SEQ ID NO: 198) and ASGPR-H2 (SEQ ID NO: 199) subunits, in a ratio of 2:1 respectively, using Lipofectamine 3000 (Invitrogen). ASGPR-H1 and ASGPR-H2 were cloned into the plasmid pCXND3 which uses the CAG promoter and encodes neomycin antibiotic resistant gene. Antibiotic resistant cells were enriched further for high expression of ASGPR by cell sorting on FACSAria III (BD). The enriched cells were then isolated by limiting dilution and plating, and identified as single cell clones by microscopy (Solentim, Cell Metric CLD). To generate cells expressing both ASGPR and eGFP-Nanoluc LgBiT, and cells expressing ASGPR and AKAP1-eGFP-NanoLuc LgBiT, HeLa cells stably expressing ASGPR were nucleofected using Cell Line Optimization 4D-Nucleofector™ X Kit (Lonza) and 4D-Nucleofector Core unit and 4D-Nucleofector X unit (Lonza), using the preset nucleofection program for HeLa. The LgBiT constructs were cloned into the plasmid pCXZD1 which uses the CAG promoter and encodes zeocin antibiotic resistant gene. The first was NanoLuc LgBit conjugated to the C-terminal of eGFP (eGFP-NanoLuc LgBit; SEQ ID NO: 200). The second was with an additional mitochondrial kinase anchoring protein 1 (AKAP1) conjugated to the N-terminal of eGFP (AKAP1-eGFP-NanoLuc LgBit; SEQ ID NO: 196. The antibiotic resistant cells were enriched further for high expression of eGFP by cell sorting on FACSAria III (BD). The enriched cells were then isolated by limiting dilution and plating, and identified as single cell clones by microscopy (Solentim, Cell Metric CLD).

HeLa cells were cultured in a standard culture media of Minimum Essential Media (Gibco) with 10% v/v Fetal Bovine Serum, 1% v/v MEM Non-Essential Amino Acids Solution (Gibco), 1% v/v Sodium Pyruvate (Gibco) and 1% v/v Penicillin-Streptomycin (Gibco). HeLa clones expressing Asialoglycoprotein receptor (ASGPR) were cultured with standard culture media supplemented with 1 mg/mL Geneticin (G418 Sulfate) (Gibco). HeLa cells expressing ASGPR and eGFP-NanoLuc LgBiT, or ASGPR and AKAP1-eGFP-NanoLuc LgBiT, were cultured in standard culture media with 1 mg/mL Geneticin and 600 micro g/mL Zeocin (Invivogen). All cell types were cultured in a humidified 5% CO₂, 37 degrees C. incubator.

### (3) Assay Set Conditions

HeLa cells expressing ASGPR and eGFP-NanoLuc LgBiT (hereafter termed as HeLa-Nluc), and HeLa cells expressing ASGPR and AKAP1-eGFP-Nanoluc LgBiT (hereafter termed as HeLa-AKAP1_Nluc) were rinsed with DPBS, no calcium, no magnesium (Gibco). The cells were then trypsinized with 0.25% Trypsin-EDTA (Gibco) at room temperature until detached from culture flask. Trypsin was then neutralized in excess culture media containing 10% FBS. Cells were pelleted by centrifugation at 200×g for 5 minutes at 4 degrees C., and rinsed twice with chilled Opti-MEM (Gibco). The cells were suspended in chilled Opti-MEM at a concentration of 1×10⁶ cells/mL, and kept cold for up to 30 minutes before use. Antibodies to be tested were diluted in Histidine Buffer, 20 mM His-HCl+150 mM NaCl (Nacalai Tesque), to 11-times of the test concentrations. 5-times substrate from the NanoLuc Live Cell Assay System (Promega, Cat #N2011) was prepared at room temperature. 1×10⁵ cells were transferred at 100 micro L per well (Perkin Elmer, Cat #6005688), and 10 micro L of 11-times antibodies were added directly into cells and mixed by pipette. 5-times substrate was then added to the cells and antibody mixture immediately after mixing.

The plate was immediately placed in a pre-warmed 37 degrees C. luminescence plate reader (Promega, Glomax Explorer). A 2-step read cycle was done every 5 minutes for 12 cycles—the plate was shaken for 1 minute at 400 rpm, before reading luminescence. Fold change with reference to a buffer well was summarized in Tables 4-8. The variants showing superior fold change compared to the parental antibody were shown in bold.

### Reference Example 2: Characterization of Extracellular Matrix (ECM) Binding of Antibodies

The binding of each antibody to ECM (extracellular matrix) was evaluated by the following procedures with reference to WO2012093704 A1: ECM Phenol red free (BD Matrigel #356237) was diluted to 2 mg/mL with TBS and 5 micro L was added to the center of each well of an ECL assay plate (L15XB-3, MSD K.K., high bind) cooled on ice. Then, the coated plate was sealed and kept overnight at 4 degrees C. The assay was then conducted at room temperature the following day. 150 micro L of ECL blocking buffer (PBS supplemented with 0.5% BSA and 0.05% Tween 20) was added to each well of the coated plate, and the plate was left standing at room temperature for 2 hours or longer. Next, the blocking buffer was removed. Each antibody sample was diluted to 9 micro g/mL with ACES-T and further diluted to 3 micro g/mL with ECLDB (PBS supplemented with 0.1% BSA and 0.01% Tween 20). 50 micro L of the diluted antibody solution was added to each well and incubated at 30 degrees C. for 1 hour with shaking at 600 rpm. The samples were then removed by tapping the inverted plate. Next, 50 micro L of glutaraldehyde was added to each well to fix the samples and incubated at room temperature for 10 minutes. The wells were then washed thrice with PBS-T (PBS supplemented with 0.05% Tween 20). A secondary antibody was diluted to 1 micro g/mL with ECLDB and 50 micro L of the secondary antibody solution was added to each well of the plate. The plate was sealed and incubated at 30 degrees C. for 1 hour at 600 rpm while shielded from light. Next, the secondary antibody solution was removed. The plate was washed thrice again with PBS-T before adding READ buffer (MSD K.K.), which was added at 150 micro L/well, followed by immediate detection of luminescence signal using Sector Imager 2400 (MSD K.K.).

### Reference Example 3: Cell Line Construction for Imaging Analysis

To generate HeLa cells stably expressing human ASGPR (HeLa/ASGPR), cells were transfected with plasmids encoding human ASGPR-H1 (SEQ ID NO: 198) and ASGPR-H2 (SEQ ID: 199) protein subunits, in a ratio of 2:1 respectively, using Lipofectamine 3000 (Theromo Fisher Scientific). ASGPR-H1 and ASGPR-H2 were cloned into the plasmid pCXND3 which uses the CAG promoter and encodes neomycin antibiotic resistant gene. Antibiotic resistant cells were enriched further for high expression of ASGPR by cell sorting on FACSAria III (Beckton Dickinson). The enriched cells were then isolated by limiting dilution and plating, and identified as single cell clones by microscopy (Solentim, Cell Metric CLD).

Gene encoding the BirA protein (SEQ ID NO: 201) was cloned into the plasmid pCXZD1 which uses the CAG promoter and encodes zeocin antibiotic resistant gene. HeLa/ASGPR cells were transfected with pCXZD1 BirA plasmid using Lipofectamine 3000 (Thermo Fisher Scientific). Antibiotic resistant cells were enriched further for high expression of BirA by cell sorting on FACSAria IIu sorter (Beckton Dickinson). The enriched cells were then isolated by limiting dilution and plating, and identified as single cell clones by microscopy. Selected clones were then expanded. Flow cytometry analysis using FACS Verse (Beckton Dickinson) with anti-ASGPR antibody and Goat Anti-Human IgG-Alexa Fluor 647 (Southern Biotech, Cat #2040-31) confirmed ASGPR expression. BirA expression was also confirmed by capillary immunoassay using Simple Western Wes (Protein Simple). This HeLa cell line stably expressing ASGPR and BirA (HeLa/ASGPR/BirA) was used for imaging analysis.

For assays, HeLa/ASGPR/BirA cells were cultured in Minimum Essential Medium Eagle (Sigma, Cat #M4655-500ML) containing 10% FBS (Sigma Aldrich, Cat #172012-500ML), Penicillin-Streptomycin (Gibco, Cat #15140-122), 750 micro g/mL Geneticin (Thermo Fisher Scientific, Cat #10131-027) and 100 micro g/mL of zeocin (Thermo Fisher Scientific, Cat #R25001).

Sequence Listing C1-2028Psq.txt

1. A cytosol-penetrating antibody or antigen-binding fragment thereof, comprising a light chain variable region comprising CDRL1, CDRL2, and CDRL3 of the amino acid sequence set forth in SEQ ID NO: 4, wherein the light chain variable region comprises one or more amino acid substitutions selected from the group consisting of (position according to Kabat numbering):
(a) substitution of amino acid K at position 24 with A, D, E, F, G, H, I, L, N, Q, R, S, T, V or Y;
(b) substitution of amino acid S at position 25 with A, D, E, F, I, M, N, T, V or Y;
(c) substitution of amino acid S at position 26 with A, D, E, F, G, H, I, K, M, N, P, R, T or V;
(d) substitution of amino acid Q at position 27 with A, D, E, F, H, I, L, M, S, or Y;
(e) substitution of amino acid S at position 27a with D;
(f) substitution of amino acid L at position 27b with I, P, Q, T or V;
(g) substitution of amino acid F at position 27c with A, D, E, I, M, N, S, W or Y;
(h) substitution of amino acid N at position 27d with A, D, E, F, H, N or Q;
(i) substitution of amino acid S at position 27e with A, D, E, I, L, M, N, P, Q, T, V or Y;
(j) substitution of amino acid R at position 27f with A, E, F, G, H, I, K, L, M, N, Q, S, T, V, W or Y;
(k) substitution of amino acid T at position 28 with E, H, I, Q, S, V, W or Y;
(l) substitution of amino acid R at position 29 with A, D, E, F, G, H, I, K, L, M, N, P, Q, S, T, V, W or Y;
(m) substitution of amino acid K at position 30 with A, D, E, F, H, I, L, M, N, P, Q, S, T, V, W or Y;
(n) substitution of amino acid N at position 31 with D, M, S or T;
(o) substitution of amino acid Y at position 32 with D, E, G, H or Q;
(p) substitution of amino acid Q at position 89 with H, I, T or Y;
(q) substitution of amino acid Y at position 91 with E, F or N;
(r) substitution of amino acid Y at position 93 with I, L, M, N, P, Q, S, T, V or W;
(s) substitution of amino acid H at position 94 with A, D, E, F, G, I, L, M, N, P, Q, R, S, T, V, W or Y;
(t) substitution of amino acid M at position 95 with A, D, E, F, G, H, I, K, L, N, P, Q, R, S, T, V, W or Y;
(u) substitution of amino acid Y at position 96 with A, D, E, F, G, H, K, M, N, P, Q, S, T or V; and
(v) substitution of amino acid T at position 97 with A, D, E, G, H, K, P, Q, R, S or V;
in the amino acid sequence set forth in SEQ ID NO: 4.

2. The cytosol-penetrating antibody or antigen-binding fragment thereof of claim 1, wherein said one or more amino acid substitutions enhance the cytosol-penetrating activity of the antibody or fragment thereof as compared to a reference antibody or fragment thereof without said substitutions and comprising a light chain variable region having the amino acid sequence set forth in SEQ ID NO: 4.

3. The cytosol-penetrating antibody or antigen-binding fragment thereof of claim 1, wherein each of the CDRL1 and CDRL3 in the light chain variable region comprises two or more amino acid substitutions selected from the group defined in claim 1 (a) to (v).

4. The cytosol-penetrating antibody or antigen-binding fragment thereof of claim 1, wherein the light chain variable region comprises amino acid substitutions selected from the group consisting of (position according to Kabat numbering):
(a) S27eD/R27fK;
(b) S27eD/R27fH;
(c) N27dE/S27eD;
(d) S27eD/T28D;
(e) S27eD/R29E;
(f) S27eD/R29S;
(g) K24N/S27eD;
(h) N27dD/R27fK;
(i) N27dE/R27fK;
(j) R27fK/T28D;
(k) R27fK/R29E;
(l) R27fK/R29S;
(m) K24N/R27fK;
(n) K24N/N27dD;
(o) N27dD/T28D;
(p) N27dD/R29E;
(q) N27dD/R29S;
(r) S27eD/Q89H;
(s) N27dD/Q89H;
(t) R27fK/Q89H;
(u) S27eD/Y91E;
(v) N27dD/Y91E;
(w) R27fK/Y91E;
(x) Q89H/H94P;
(y) Y91E/H94P;
(z) H94P/M95N;
(aa) H94P/M95D;
(bb) H94P/M95P;
(cc) H94E/Y96P;
(dd) H94E/M95N;
(ee) H94E/M95D;
(ff) H94E/M95P;
(gg) N27dE/R27fK/Q89H/H94E/M95P;
(hh) N27dE/R27fK/Q89Y/H94E/M95P;
(ii) N27dE/R27fK/Y91E/H94E/M95P;
(jj) N27dE/R27fK/Y91F/H94E/M95P;
(kk) N27dE/R27fS/Q89H/H94E/M95P;
(ll) N27dE/R27fS/Q89Y/H94E/M95P;
(mm) N27dE/R27fS/Y91E/H94E/M95P;
(nn) N27dE/R27fS/Y91F/H94E/M95P;
(oo) N27dE/R27fS/Q89H/H94Q/M95P;
(pp) N27dE/R27fS/Q89Y/H94Q/M95P;
(qq) N27dE/R27fS/Y91E/H94Q/M95P;
(rr) N27dE/R27fS/Y91F/H94Q/M95P;
(ss) N27dE/R27fV/Q89H/H94E/M95P;
(tt) N27dE/R27fV/Q89Y/H94E/M95P;
(uu) N27dE/R27fV/Y91E/H94E/M95P;
(vv) N27dE/R27fV/Y91F/H94E/M95P;
(ww) N27dE/R29S/Q89H/H94E/M95P;
(xx) N27dE/R29S/Q89Y/H94E/M95P;
(yy) N27dE/K30L/Q89H/H94E/M95P;
(zz) N27dE/K30L/Q89Y/H94E/M95P;
(aaa) S27eD/R27fK/Y91E/H94E/M95P;
(bbb) S27eD/R27fK/Y91F/H94E/M95P;
(ccc) S27eD/R27fK/Y91E/H94Q/M95P;
(ddd) S27eD/R27fK/Y91F/H94Q/M95P;
(eee) S27eD/R27fK/Y91E/H94Q;
(fff) S27eD/R27fK/Y91F/H94Q;
(ggg) S27eE/R27fK/Y91E/H94E/M95P;
(hhh) S27eE/R27fK/Y91F/H94E/M95P;
(iii) S27eE/R27fK/Y91E/H94Q/M95P;
(jjj) S27eE/R27fK/Y91F/H94Q/M95P;
(kkk) R27fS/R29S/Q89H/H94E/M95P;
(lll) R27fS/R29S/Q89Y/H94E/M95P;
(mmm) R27fS/R29S/Y91E/H94E/M95P;
(nnn) R27fS/R29S/Y91F/H94E/M95P;
(ooo) R27fS/R29S/Q89H/H94Q/M95P;
(ppp) R27fS/R29S/Q89Y/H94Q/M95P;
(qqq) R27fS/R29S/Y91E/H94Q/M95P;
(rrr) R27fS/R29S/Y91F/H94Q/M95P;
(sss) N27dE/R27fS/R29S/Q89Y/H94Q/M95P; and
(ttt) N27dE/R27fS/R29S/Y91F/H94Q/M95P.

5. The cytosol-penetrating antibody or antigen-binding fragment thereof of claim 1, wherein the light chain variable region comprises a human framework region (FR).

6. The cytosol-penetrating antibody or antigen-binding fragment thereof of claim 5, wherein the light chain variable region comprises amino acid I at position 2, and/or amino acid Q at position 3 according to Kabat numbering.

7. The cytosol-penetrating antibody or antigen-binding fragment thereof of claim 5, wherein the light chain variable region comprises one, two, three, or four of the FR domains (FR1, FR2, FR3, and FR4) comprised in SEQ ID NO: 123.

8. The cytosol-penetrating antibody or antigen-binding fragment thereof of claim 1, wherein the light chain variable region further comprises one, two, three or more amino acid substitutions in the CDRL2.

9. The cytosol-penetrating antibody or antigen-binding fragment thereof of claim 1, wherein the light chain variable region further comprises one, two, three or more amino acid substitutions selected from the group consisting of (position according to Kabat numbering):
(a) substitution of amino acid W at position 50 with A, G, I, T or V;
(b) substitution of amino acid S at position 52 with F or I;
(c) substitution of amino acid T at position 53 with N or Y; and
(d) substitution of amino acid R at position 54 with K or V;
in the CDRL2 of the amino acid sequence set forth in SEQ ID NO: 4.

10. The cytosol-penetrating antibody or antigen-binding fragment thereof of claim 1, wherein the light chain variable region comprises amino acid substitutions selected from the group consisting of (position according to Kabat numbering):
(a) L2I/V3Q/S27eD/R27fK/W50A/Y91F/H94Q;
(b) L2I/V3Q/S27eD/R27fK/W50G/Y91F/H94Q;
(c) L2I/V3Q/S27eD/R27fK/W501/Y91F/H94Q;
(d) L2I/V3Q/S27eD/R27fK/W50T/Y91F/H94Q;
(e) L2I/V3Q/S27eD/R27fK/W50V/Y91F/H94Q;
(f) L2I/V3Q/S27eD/R27fK/S52F/Y91F/H94Q;
(g) L2I/V3Q/S27eD/R27fK/S52I/Y91F/H94Q;
(h) L2I/V3Q/S27eD/R27fK/T53N/Y91F/H94Q;
(i) L2I/V3Q/S27eD/R27fK/T53Y/Y91F/H94Q;
(j) L2I/V3Q/S27eD/R27fK/R54K/Y91F/H94Q;
(k) L2I/V3Q/S27eD/R27fK/W50I/S52F/Y91F/H94Q;
(l) L2I/V3Q/S27eD/R27fK/W50I/T53N/Y91F/H94Q; and
(m) L2I/V3Q/S27eD/R27fK/W50I/R54V/Y91F/H94Q;
in the amino acid sequence of SEQ ID NO: 4.

11. The cytosol-penetrating antibody or antigen-binding fragment thereof of claim 1, comprising a light chain variable region having an amino acid sequence selected from the group consisting of SEQ ID NOs: 14-150 and 161-173.

12. The cytosol-penetrating antibody or antigen-binding fragment thereof of claim 11, wherein the cytosol-penetrating antibody or antigen-binding fragment thereof comprises a light chain variable region having an amino acid sequence selected from the group consisting of SEQ ID NOs: 123-150.

13. The cytosol-penetrating antibody or antigen-binding fragment thereof of claim 11, wherein the cytosol-penetrating antibody or antigen-binding fragment thereof comprises a light chain variable region having an amino acid sequence selected from the group consisting of SEQ ID NOs: 161-173.

14. A multifunctional antibody, comprising a cell surface antigen-binding domain, a cytosolic antigen-binding domain, and a cytosol-penetrating domain; wherein the cytosol-penetrating domain comprises the light chain variable region of claim 1.

15. A pharmaceutical composition comprising the cytosol-penetrating antibody or antigen-binding fragment thereof of claim 1 and a pharmaceutically acceptable carrier.

16. The cytosol-penetrating antibody or antigen-binding fragment thereof of claim 1, which is fused to or conjugated with a bioactive molecule.

17. The cytosol-penetrating antibody or antigen-binding fragment thereof of claim 16, wherein the bioactive molecule is selected from the group consisting of a peptide, a protein, a toxin, an antibody, an antibody fragment, an RNA, a DNA, a small molecule drug, a nanoparticle, and a liposome.

18. A nucleic acid encoding the cytosol-penetrating antibody or antigen-binding fragment thereof of claim 1.

19. A host cell comprising the nucleic acid of claim 18.

20. A method of producing the cytosol-penetrating antibody or antigen-binding fragment thereof, comprising culturing the host cell of claim 19 under conditions suitable for expression of the antibody or fragment thereof, and optionally recovering the antibody or fragment thereof from the host cell or host cell culture medium.

21. A method of delivering a bioactive molecule into a cell cytosol comprising contacting the cell with the cytosol-penetrating antibody or antigen-binding fragment thereof of claim 16.


SEQUENCE LISTING (203 sequences)

SEQ ID NO: 1 - protein, 113 aa, Artificial Sequence
  An artificially synthesized sequence
DIVLTQSPATLSLSPGERATLSCKSSQSLFNSRTRKNYLAWYQQKPGKAPKLLIYWASTRESGVPSRFSGSGSGTDFTLTISSLQPEDFATYYCQQYYYHMYTFGQGTKVEIK

SEQ ID NO: 2 - protein, 113 aa, Artificial Sequence
  An artificially synthesized sequence
DLVMTQSPATLSLSPGERATLSCKSSQSLFNSRTRKNYLAWYQQKPGKAPKLLIYWASTRESGVPSRFSGSGSGTDFTLTISSLQPEDFATYYCQQYYYHMYTFGQGTKVEIK

SEQ ID NO: 3 - protein, 113 aa, Artificial Sequence
  An artificially synthesized sequence
DLVMTQSPSSLSASVGDRVTITCKSSQSLFNSRTRKNYLAWYQQKPGQAPRLLIYWASTRESGVPSRFSGSGSGTDFTLTISSLQPEDFATYYCQQYYYHMYTFGQGTKVEIK

SEQ ID NO: 4 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYHMYTFGQGTKVEIK

SEQ ID NO: 5 - protein, 113 aa, Artificial Sequence
  An artificially synthesized sequence
DIVLTQSPATLSLSPGERATLSCKSSQSLFNSRTRKNYLAWYQQKPGQAPRLLIYWASTRESGVPSRFSGSGSGTDFTLTISSLQPEDFATYYCQQYYYHMYTFGQGTKVEIK

SEQ ID NO: 6 - protein, 113 aa, Artificial Sequence
  An artificially synthesized sequence
DLVMTQSPATLSLSPGERATLSCKSSQSLFNSRTRKNYLAWYQQKPGQAPRLLIYWASTRESGVPSRFSGSGSGTDFTLTISSLQPEDFATYYCQQYYYHMYTFGQGTKVEIK

SEQ ID NO: 7 - protein, 113 aa, Artificial Sequence
  An artificially synthesized sequence
DIVLTQSPATLSLSPGERATLSCKSSQSLFNSRTRKNYLAWYQQKPGKAPKLLIYWASTRESGVPARFSGSGSGTDFTLTISSLEPEDFAVYYCQQYYYHMYTFGQGTKVEIK

SEQ ID NO: 8 - protein, 113 aa, Artificial Sequence
  An artificially synthesized sequence
DLVMTQSPATLSLSPGERATLSCKSSQSLFNSRTRKNYLAWYQQKPGKAPKLLIYWASTRESGVPARFSGSGSGTDFTLTISSLEPEDFAVYYCQQYYYHMYTFGQGTKVEIK

SEQ ID NO: 9 - protein, 113 aa, Artificial Sequence
  An artificially synthesized sequence
DIVLTQSPATLSLSPGERATLSCKSSQSLFNSRTRKNYLAWYQQKPGQAPRLLIYWASTRESGVPARFSGSGSGTDFTLTISSLEPEDFAVYYCQQYYYHMYTFGQGTKVEIK

SEQ ID NO: 10 - protein, 113 aa, Artificial Sequence
  An artificially synthesized sequence
DLVMTQSPATLSLSPGERATLSCKSSQSLFNSRTRKNYLAWYQQKPGQAPRLLIYWASTRESGVPARFSGSGSGTDFTLTISSLEPEDFAVYYCQQYYYHMYTFGQGTKVEIK

SEQ ID NO: 11 - protein, 113 aa, Artificial Sequence
  An artificially synthesized sequence
DLVMTQSPSSLSASVGDRVTITCKSSQSLFNSRTRKNYLAWYQQKPGQAPRLLIYWASTRESGVPARFSGSGSGTDFTLTISSLEPEDFAVYYCQQYYYHMYTFGQGTKVEIK

SEQ ID NO: 12 - protein, 107 aa, Artificial Sequence [sequence omitted]

SEQ ID NO: 13 - protein, 467 aa, Artificial Sequence
  An artificially synthesized sequence
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGAYKRGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGSVTGYRLFEEIL

SEQ ID NO: 14 - protein, 113 aa, Artificial Sequence
  An artificially synthesized sequence
DLVMTQSPSSLSASVGDRVTITCNSSQSLFNSRTRKNYLAWYQQKPGKAPKLLIYWASTRESGVPARFSGSGSGTDFTLTISSLEPEDFAVYYCQQYYYHMYTFGQGTKVEIK

SEQ ID NO: 15 - protein, 113 aa, Artificial Sequence
  An artificially synthesized sequence
DLVMTQSPSSLSASVGDRVTITCKSSESLFNSRTRKNYLAWYQQKPGKAPKLLIYWASTRESGVPARFSGSGSGTDFTLTISSLEPEDFAVYYCQQYYYHMYTFGQGTKVEIK

SEQ ID NO: 16 - protein, 113 aa, Artificial Sequence
  An artificially synthesized sequence
DLVMTQSPSSLSASVGDRVTITCKSSQDLFNSRTRKNYLAWYQQKPGKAPKLLIYWASTRESGVPARFSGSGSGTDFTLTISSLEPEDFAVYYCQQYYYHMYTFGQGTKVEIK

SEQ ID NO: 17 - protein, 113 aa, Artificial Sequence
  An artificially synthesized sequence
DLVMTQSPSSLSASVGDRVTITCKSSQSLFDSRTRKNYLAWYQQKPGKAPKLLIYWASTRESGVPARFSGSGSGTDFTLTISSLEPEDFAVYYCQQYYYHMYTFGQGTKVEIK

SEQ ID NO: 18 - protein, 113 aa, Artificial Sequence
  An artificially synthesized sequence
DLVMTQSPSSLSASVGDRVTITCKSSQSLFESRTRKNYLAWYQQKPGKAPKLLIYWASTRESGVPARFSGSGSGTDFTLTISSLEPEDFAVYYCQQYYYHMYTFGQGTKVEIK

SEQ ID NO: 19 - protein, 113 aa, Artificial Sequence
  An artificially synthesized sequence
DLVMTQSPSSLSASVGDRVTITCKSSQSLFNDRTRKNYLAWYQQKPGKAPKLLIYWASTRESGVPARFSGSGSGTDFTLTISSLEPEDFAVYYCQQYYYHMYTFGQGTKVEIK

SEQ ID NO: 20 - protein, 113 aa, Artificial Sequence
  An artificially synthesized sequence
DLVMTQSPSSLSASVGDRVTITCKSSQSLFNERTRKNYLAWYQQKPGKAPKLLIYWASTRESGVPARFSGSGSGTDFTLTISSLEPEDFAVYYCQQYYYHMYTFGQGTKVEIK

SEQ ID NO: 21 - protein, 113 aa, Artificial Sequence
  An artificially synthesized sequence
DLVMTQSPSSLSASVGDRVTITCKSSQSLFNSDTRKNYLAWYQQKPGKAPKLLIYWASTRESGVPARFSGSGSGTDFTLTISSLEPEDFAVYYCQQYYYHMYTFGQGTKVEIK

SEQ ID NO: 22 - protein, 113 aa, Artificial Sequence
  An artificially synthesized sequence
DLVMTQSPSSLSASVGDRVTITCKSSQSLFNSHTRKNYLAWYQQKPGKAPKLLIYWASTRESGVPARFSGSGSGTDFTLTISSLEPEDFAVYYCQQYYYHMYTFGQGTKVEIK

SEQ ID NO: 23 - protein, 113 aa, Artificial Sequence
  An artificially synthesized sequence
DLVMTQSPSSLSASVGDRVTITCKSSQSLFNSKTRKNYLAWYQQKPGKAPKLLIYWASTRESGVPARFSGSGSGTDFTLTISSLEPEDFAVYYCQQYYYHMYTFGQGTKVEIK

SEQ ID NO: 24 - protein, 113 aa, Artificial Sequence
  An artificially synthesized sequence
DLVMTQSPSSLSASVGDRVTITCKSSQSLFNSLTRKNYLAWYQQKPGKAPKLLIYWASTRESGVPARFSGSGSGTDFTLTISSLEPEDFAVYYCQQYYYHMYTFGQGTKVEIK

SEQ ID NO: 25 - protein, 113 aa, Artificial Sequence
  An artificially synthesized sequence
DLVMTQSPSSLSASVGDRVTITCKSSQSLFNSSTRKNYLAWYQQKPGKAPKLLIYWASTRESGVPARFSGSGSGTDFTLTISSLEPEDFAVYYCQQYYYHMYTFGQGTKVEIK

SEQ ID NO: 26 - protein, 113 aa, Artificial Sequence
  An artificially synthesized sequence
DLVMTQSPSSLSASVGDRVTITCKSSQSLFNSVTRKNYLAWYQQKPGKAPKLLIYWASTRESGVPARFSGSGSGTDFTLTISSLEPEDFAVYYCQQYYYHMYTFGQGTKVEIK

SEQ ID NO: 27 - protein, 113 aa, Artificial Sequence
  An artificially synthesized sequence
DLVMTQSPSSLSASVGDRVTITCKSSQSLFNSRDRKNYLAWYQQKPGKAPKLLIYWASTRESGVPARFSGSGSGTDFTLTISSLEPEDFAVYYCQQYYYHMYTFGQGTKVEIK

SEQ ID NO: 28 - protein, 113 aa, Artificial Sequence
  An artificially synthesized sequence
DLVMTQSPSSLSASVGDRVTITCKSSQSLFNSRTEKNYLAWYQQKPGKAPKLLIYWASTRESGVPARFSGSGSGTDFTLTISSLEPEDFAVYYCQQYYYHMYTFGQGTKVEIK

SEQ ID NO: 29 - protein, 113 aa, Artificial Sequence
  An artificially synthesized sequence
DLVMTQSPSSLSASVGDRVTITCKSSQSLFNSRTGKNYLAWYQQKPGKAPKLLIYWASTRESGVPARFSGSGSGTDFTLTISSLEPEDFAVYYCQQYYYHMYTFGQGTKVEIK

SEQ ID NO: 30 - protein, 113 aa, Artificial Sequence
  An artificially synthesized sequence
DLVMTQSPSSLSASVGDRVTITCKSSQSLFNSRTMKNYLAWYQQKPGKAPKLLIYWASTRESGVPARFSGSGSGTDFTLTISSLEPEDFAVYYCQQYYYHMYTFGQGTKVEIK

SEQ ID NO: 31 - protein, 113 aa, Artificial Sequence
  An artificially synthesized sequence
DLVMTQSPSSLSASVGDRVTITCKSSQSLFNSRTSKNYLAWYQQKPGKAPKLLIYWASTRESGVPARFSGSGSGTDFTLTISSLEPEDFAVYYCQQYYYHMYTFGQGTKVEIK

SEQ ID NO: 32 - protein, 113 aa, Artificial Sequence
  An artificially synthesized sequence
DLVMTQSPSSLSASVGDRVTITCKSSQSLFNSRTRLNYLAWYQQKPGKAPKLLIYWASTRESGVPARFSGSGSGTDFTLTISSLEPEDFAVYYCQQYYYHMYTFGQGTKVEIK

SEQ ID NO: 33 - protein, 113 aa, Artificial Sequence
  An artificially synthesized sequence
DLVMTQSPSSLSASVGDRVTITCKSSQSLFNSRTRQNYLAWYQQKPGKAPKLLIYWASTRESGVPARFSGSGSGTDFTLTISSLEPEDFAVYYCQQYYYHMYTFGQGTKVEIK

SEQ ID NO: 34 - protein, 113 aa, Artificial Sequence
  An artificially synthesized sequence
DLVMTQSPSSLSASVGDRVTITCKSSQSLFNSRTRKNELAWYQQKPGKAPKLLIYWASTRESGVPARFSGSGSGTDFTLTISSLEPEDFAVYYCQQYYYHMYTFGQGTKVEIK

SEQ ID NO: 35 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCHQYYYHMYTFGQGTKVEIK

SEQ ID NO: 36 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCIQYYYHMYTFGQGTKVEIK

SEQ ID NO: 37 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCTQYYYHMYTFGQGTKVEIK

SEQ ID NO: 38 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCYQYYYHMYTFGQGTKVEIK

SEQ ID NO: 39 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQEYYHMYTFGQGTKVEIK

SEQ ID NO: 40 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQFYYHMYTFGQGTKVEIK

SEQ ID NO: 41 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQNYYHMYTFGQGTKVEIK

SEQ ID NO: 42 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYEHMYTFGQGTKVEIK

SEQ ID NO: 43 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYEMYTFGQGTKVEIK

SEQ ID NO: 44 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYPMYTFGQGTKVEIK

SEQ ID NO: 45 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYQMYTFGQGTKVEIK

SEQ ID NO: 46 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYHDYTFGQGTKVEIK

SEQ ID NO: 47 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYHNYTFGQGTKVEIK

SEQ ID NO: 48 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYHPYTFGQGTKVEIK

SEQ ID NO: 49 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYHMETFGQGTKVEIK

SEQ ID NO: 50 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYHMPTFGQGTKVEIK

SEQ ID NO: 51 - protein, 113 aa, Artificial Sequence
  An artificially synthesized sequence
DLVMTQSPSSLSASVGDRVTITCKSSQSLFNDKTRKNYLAWYQQKPGKAPKLLIYWASTRESGVPARFSGSGSGTDFTLTISSLEPEDFAVYYCQQYYYHMYTFGQGTKVEIK

SEQ ID NO: 52 - protein, 113 aa, Artificial Sequence
  An artificially synthesized sequence
DLVMTQSPSSLSASVGDRVTITCKSSQSLFNDHTRKNYLAWYQQKPGKAPKLLIYWASTRESGVPARFSGSGSGTDFTLTISSLEPEDFAVYYCQQYYYHMYTFGQGTKVEIK

SEQ ID NO: 53 - protein, 113 aa, Artificial Sequence
  An artificially synthesized sequence
DLVMTQSPSSLSASVGDRVTITCKSSQSLFEDRTRKNYLAWYQQKPGKAPKLLIYWASTRESGVPARFSGSGSGTDFTLTISSLEPEDFAVYYCQQYYYHMYTFGQGTKVEIK

SEQ ID NO: 54 - protein, 113 aa, Artificial Sequence
  An artificially synthesized sequence
DLVMTQSPSSLSASVGDRVTITCKSSQSLFNDRDRKNYLAWYQQKPGKAPKLLIYWASTRESGVPARFSGSGSGTDFTLTISSLEPEDFAVYYCQQYYYHMYTFGQGTKVEIK

SEQ ID NO: 55 - protein, 113 aa, Artificial Sequence
  An artificially synthesized sequence
DLVMTQSPSSLSASVGDRVTITCKSSQSLFNDRTEKNYLAWYQQKPGKAPKLLIYWASTRESGVPARFSGSGSGTDFTLTISSLEPEDFAVYYCQQYYYHMYTFGQGTKVEIK

SEQ ID NO: 56 - protein, 113 aa, Artificial Sequence
  An artificially synthesized sequence
DLVMTQSPSSLSASVGDRVTITCKSSQSLFNDRTSKNYLAWYQQKPGKAPKLLIYWASTRESGVPARFSGSGSGTDFTLTISSLEPEDFAVYYCQQYYYHMYTFGQGTKVEIK

SEQ ID NO: 57 - protein, 113 aa, Artificial Sequence
  An artificially synthesized sequence
DLVMTQSPSSLSASVGDRVTITCNSSQSLFNDRTRKNYLAWYQQKPGKAPKLLIYWASTRESGVPARFSGSGSGTDFTLTISSLEPEDFAVYYCQQYYYHMYTFGQGTKVEIK

SEQ ID NO: 58 - protein, 113 aa, Artificial Sequence
  An artificially synthesized sequence
DLVMTQSPSSLSASVGDRVTITCKSSQSLFDSKTRKNYLAWYQQKPGKAPKLLIYWASTRESGVPARFSGSGSGTDFTLTISSLEPEDFAVYYCQQYYYHMYTFGQGTKVEIK

SEQ ID NO: 59 - protein, 113 aa, Artificial Sequence
  An artificially synthesized sequence
DLVMTQSPSSLSASVGDRVTITCKSSQSLFESKTRKNYLAWYQQKPGKAPKLLIYWASTRESGVPARFSGSGSGTDFTLTISSLEPEDFAVYYCQQYYYHMYTFGQGTKVEIK

SEQ ID NO: 60 - protein, 113 aa, Artificial Sequence
  An artificially synthesized sequence
DLVMTQSPSSLSASVGDRVTITCKSSQSLFNSKDRKNYLAWYQQKPGKAPKLLIYWASTRESGVPARFSGSGSGTDFTLTISSLEPEDFAVYYCQQYYYHMYTFGQGTKVEIK

SEQ ID NO: 61 - protein, 113 aa, Artificial Sequence
  An artificially synthesized sequence
DLVMTQSPSSLSASVGDRVTITCKSSQSLFNSKTEKNYLAWYQQKPGKAPKLLIYWASTRESGVPARFSGSGSGTDFTLTISSLEPEDFAVYYCQQYYYHMYTFGQGTKVEIK

SEQ ID NO: 62 - protein, 113 aa, Artificial Sequence
  An artificially synthesized sequence
DLVMTQSPSSLSASVGDRVTITCKSSQSLFNSKTSKNYLAWYQQKPGKAPKLLIYWASTRESGVPARFSGSGSGTDFTLTISSLEPEDFAVYYCQQYYYHMYTFGQGTKVEIK

SEQ ID NO: 63 - protein, 113 aa, Artificial Sequence
  An artificially synthesized sequence
DLVMTQSPSSLSASVGDRVTITCNSSQSLFNSKTRKNYLAWYQQKPGKAPKLLIYWASTRESGVPARFSGSGSGTDFTLTISSLEPEDFAVYYCQQYYYHMYTFGQGTKVEIK

SEQ ID NO: 64 - protein, 113 aa, Artificial Sequence
  An artificially synthesized sequence
DLVMTQSPSSLSASVGDRVTITCNSSQSLFDSRTRKNYLAWYQQKPGKAPKLLIYWASTRESGVPARFSGSGSGTDFTLTISSLEPEDFAVYYCQQYYYHMYTFGQGTKVEIK

SEQ ID NO: 65 - protein, 113 aa, Artificial Sequence
  An artificially synthesized sequence
DLVMTQSPSSLSASVGDRVTITCKSSQSLFDSRDRKNYLAWYQQKPGKAPKLLIYWASTRESGVPARFSGSGSGTDFTLTISSLEPEDFAVYYCQQYYYHMYTFGQGTKVEIK

SEQ ID NO: 66 - protein, 113 aa, Artificial Sequence
  An artificially synthesized sequence
DLVMTQSPSSLSASVGDRVTITCKSSQSLFDSRTEKNYLAWYQQKPGKAPKLLIYWASTRESGVPARFSGSGSGTDFTLTISSLEPEDFAVYYCQQYYYHMYTFGQGTKVEIK

SEQ ID NO: 67 - protein, 113 aa, Artificial Sequence
  An artificially synthesized sequence
DLVMTQSPSSLSASVGDRVTITCKSSQSLFDSRTSKNYLAWYQQKPGKAPKLLIYWASTRESGVPARFSGSGSGTDFTLTISSLEPEDFAVYYCQQYYYHMYTFGQGTKVEIK

SEQ ID NO: 68 - protein, 113 aa, Artificial Sequence
  An artificially synthesized sequence
DLVMTQSPSSLSASVGDRVTITCKSSQSLFNDRTRKNYLAWYQQKPGKAPKLLIYWASTRESGVPARFSGSGSGTDFTLTISSLEPEDFAVYYCHQYYYHMYTFGQGTKVEIK

SEQ ID NO: 69 - protein, 113 aa, Artificial Sequence
  An artificially synthesized sequence
DLVMTQSPSSLSASVGDRVTITCKSSQSLFDSRTRKNYLAWYQQKPGKAPKLLIYWASTRESGVPARFSGSGSGTDFTLTISSLEPEDFAVYYCHQYYYHMYTFGQGTKVEIK

SEQ ID NO: 70 - protein, 113 aa, Artificial Sequence
  An artificially synthesized sequence
DLVMTQSPSSLSASVGDRVTITCKSSQSLFNSKTRKNYLAWYQQKPGKAPKLLIYWASTRESGVPARFSGSGSGTDFTLTISSLEPEDFAVYYCHQYYYHMYTFGQGTKVEIK

SEQ ID NO: 71 - protein, 113 aa, Artificial Sequence
  An artificially synthesized sequence
DLVMTQSPSSLSASVGDRVTITCKSSQSLFNDRTRKNYLAWYQQKPGKAPKLLIYWASTRESGVPARFSGSGSGTDFTLTISSLEPEDFAVYYCQQEYYHMYTFGQGTKVEIK

SEQ ID NO: 72 - protein, 113 aa, Artificial Sequence
  An artificially synthesized sequence
DLVMTQSPSSLSASVGDRVTITCKSSQSLFDSRTRKNYLAWYQQKPGKAPKLLIYWASTRESGVPARFSGSGSGTDFTLTISSLEPEDFAVYYCQQEYYHMYTFGQGTKVEIK

SEQ ID NO: 73 - protein, 113 aa, Artificial Sequence
  An artificially synthesized sequence
DLVMTQSPSSLSASVGDRVTITCKSSQSLFNSKTRKNYLAWYQQKPGKAPKLLIYWASTRESGVPARFSGSGSGTDFTLTISSLEPEDFAVYYCQQEYYHMYTFGQGTKVEIK

SEQ ID NO: 74 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCHQYYYPMYTFGQGTKVEIK

SEQ ID NO: 75 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQEYYPMYTFGQGTKVEIK

SEQ ID NO: 76 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYPNYTFGQGTKVEIK

SEQ ID NO: 77 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYPDYTFGQGTKVEIK

SEQ ID NO: 78 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYPPYTFGQGTKVEIK

SEQ ID NO: 79 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYEMPTFGQGTKVEIK

SEQ ID NO: 80 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYENYTFGQGTKVEIK

SEQ ID NO: 81 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYEDYTFGQGTKVEIK

SEQ ID NO: 82 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPARFSGSGSGTDFTLTISSLEPEDFAVYYCQQYYYEPYTFGQGTKVEIK

SEQ ID NO: 83 - protein, 113 aa, Artificial Sequence
  An artificially synthesized sequence
DLVMTQSPSSLSASVGDRVTITCKSSQSLFESKTRKNYLAWYQQKPGKAPKLLIYWASTRESGVPARFSGSGSGTDFTLTISSLEPEDFAVYYCHQYYYEPYTFGQGTKVEIK

SEQ ID NO: 84 - protein, 113 aa, Artificial Sequence
  An artificially synthesized sequence
DLVMTQSPSSLSASVGDRVTITCKSSQSLFESKTRKNYLAWYQQKPGKAPKLLIYWASTRESGVPARFSGSGSGTDFTLTISSLEPEDFAVYYCYQYYYEPYTFGQGTKVEIK

SEQ ID NO: 85 - protein, 113 aa, Artificial Sequence
  An artificially synthesized sequence
DLVMTQSPSSLSASVGDRVTITCKSSQSLFESKTRKNYLAWYQQKPGKAPKLLIYWASTRESGVPARFSGSGSGTDFTLTISSLEPEDFAVYYCQQEYYEPYTFGQGTKVEIK

SEQ ID NO: 86 - protein, 113 aa, Artificial Sequence
  An artificially synthesized sequence
DLVMTQSPSSLSASVGDRVTITCKSSQSLFESKTRKNYLAWYQQKPGKAPKLLIYWASTRESGVPARFSGSGSGTDFTLTISSLEPEDFAVYYCQQFYYEPYTFGQGTKVEIK

SEQ ID NO: 87 - protein, 113 aa, Artificial Sequence
  An artificially synthesized sequence
DLVMTQSPSSLSASVGDRVTITCKSSQSLFESSTRKNYLAWYQQKPGKAPKLLIYWASTRESGVPARFSGSGSGTDFTLTISSLEPEDFAVYYCHQYYYEPYTFGQGTKVEIK

SEQ ID NO: 88 - protein, 113 aa, Artificial Sequence
  An artificially synthesized sequence
DLVMTQSPSSLSASVGDRVTITCKSSQSLFESSTRKNYLAWYQQKPGKAPKLLIYWASTRESGVPARFSGSGSGTDFTLTISSLEPEDFAVYYCYQYYYEPYTFGQGTKVEIK

SEQ ID NO: 89 - protein, 113 aa, Artificial Sequence
  An artificially synthesized sequence
DLVMTQSPSSLSASVGDRVTITCKSSQSLFESSTRKNYLAWYQQKPGKAPKLLIYWASTRESGVPARFSGSGSGTDFTLTISSLEPEDFAVYYCQQEYYEPYTFGQGTKVEIK

SEQ ID NO: 90 - protein, 113 aa, Artificial Sequence
  An artificially synthesized sequence
DLVMTQSPSSLSASVGDRVTITCKSSQSLFESSTRKNYLAWYQQKPGKAPKLLIYWASTRESGVPARFSGSGSGTDFTLTISSLEPEDFAVYYCQQFYYEPYTFGQGTKVEIK

SEQ ID NO: 91 - protein, 113 aa, Artificial Sequence
  An artificially synthesized sequence
DLVMTQSPSSLSASVGDRVTITCKSSQSLFESSTRKNYLAWYQQKPGKAPKLLIYWASTRESGVPARFSGSGSGTDFTLTISSLEPEDFAVYYCHQYYYQPYTFGQGTKVEIK

SEQ ID NO: 92 - protein, 113 aa, Artificial Sequence
  An artificially synthesized sequence
DLVMTQSPSSLSASVGDRVTITCKSSQSLFESSTRKNYLAWYQQKPGKAPKLLIYWASTRESGVPARFSGSGSGTDFTLTISSLEPEDFAVYYCYQYYYQPYTFGQGTKVEIK

SEQ ID NO: 93 - protein, 113 aa, Artificial Sequence
  An artificially synthesized sequence
DLVMTQSPSSLSASVGDRVTITCKSSQSLFESSTRKNYLAWYQQKPGKAPKLLIYWASTRESGVPARFSGSGSGTDFTLTISSLEPEDFAVYYCQQEYYQPYTFGQGTKVEIK

SEQ ID NO: 94 - protein, 113 aa, Artificial Sequence
  An artificially synthesized sequence
DLVMTQSPSSLSASVGDRVTITCKSSQSLFESSTRKNYLAWYQQKPGKAPKLLIYWASTRESGVPARFSGSGSGTDFTLTISSLEPEDFAVYYCQQFYYQPYTFGQGTKVEIK

SEQ ID NO: 95 - protein, 113 aa, Artificial Sequence
  An artificially synthesized sequence
DLVMTQSPSSLSASVGDRVTITCKSSQSLFESVTRKNYLAWYQQKPGKAPKLLIYWASTRESGVPARFSGSGSGTDFTLTISSLEPEDFAVYYCHQYYYEPYTFGQGTKVEIK

SEQ ID NO: 96 - protein, 113 aa, Artificial Sequence
  An artificially synthesized sequence
DLVMTQSPSSLSASVGDRVTITCKSSQSLFESVTRKNYLAWYQQKPGKAPKLLIYWASTRESGVPARFSGSGSGTDFTLTISSLEPEDFAVYYCYQYYYEPYTFGQGTKVEIK

SEQ ID NO: 97 - protein, 113 aa, Artificial Sequence
  An artificially synthesized sequence
DLVMTQSPSSLSASVGDRVTITCKSSQSLFESVTRKNYLAWYQQKPGKAPKLLIYWASTRESGVPARFSGSGSGTDFTLTISSLEPEDFAVYYCQQEYYEPYTFGQGTKVEIK

SEQ ID NO: 98 - protein, 113 aa, Artificial Sequence
  An artificially synthesized sequence
DLVMTQSPSSLSASVGDRVTITCKSSQSLFESVTRKNYLAWYQQKPGKAPKLLIYWASTRESGVPARFSGSGSGTDFTLTISSLEPEDFAVYYCQQFYYEPYTFGQGTKVEIK

SEQ ID NO: 99 - protein, 113 aa, Artificial Sequence
  An artificially synthesized sequence
DLVMTQSPSSLSASVGDRVTITCKSSQSLFESRTSKNYLAWYQQKPGKAPKLLIYWASTRESGVPARFSGSGSGTDFTLTISSLEPEDFAVYYCHQYYYEPYTFGQGTKVEIK

SEQ ID NO: 100 - protein, 113 aa, Artificial Sequence
  An artificially synthesized sequence
DLVMTQSPSSLSASVGDRVTITCKSSQSLFESRTSKNYLAWYQQKPGKAPKLLIYWASTRESGVPARFSGSGSGTDFTLTISSLEPEDFAVYYCYQYYYEPYTFGQGTKVEIK

SEQ ID NO: 101 - protein, 113 aa, Artificial Sequence
  An artificially synthesized sequence
DLVMTQSPSSLSASVGDRVTITCKSSQSLFESRTRLNYLAWYQQKPGKAPKLLIYWASTRESGVPARFSGSGSGTDFTLTISSLEPEDFAVYYCHQYYYEPYTFGQGTKVEIK

SEQ ID NO: 102 - protein, 113 aa, Artificial Sequence
  An artificially synthesized sequence
DLVMTQSPSSLSASVGDRVTITCKSSQSLFESRTRLNYLAWYQQKPGKAPKLLIYWASTRESGVPARFSGSGSGTDFTLTISSLEPEDFAVYYCYQYYYEPYTFGQGTKVEIK

SEQ ID NO: 103 - protein, 113 aa, Artificial Sequence
  An artificially synthesized sequence
DLVMTQSPSSLSASVGDRVTITCKSSQSLFNDKTRKNYLAWYQQKPGKAPKLLIYWASTRESGVPARFSGSGSGTDFTLTISSLEPEDFAVYYCQQEYYEPYTFGQGTKVEIK

SEQ ID NO: 104 - protein, 113 aa, Artificial Sequence
  An artificially synthesized sequence
DLVMTQSPSSLSASVGDRVTITCKSSQSLFNDKTRKNYLAWYQQKPGKAPKLLIYWASTRESGVPARFSGSGSGTDFTLTISSLEPEDFAVYYCQQFYYEPYTFGQGTKVEIK

SEQ ID NO: 105 - protein, 113 aa, Artificial Sequence
  An artificially synthesized sequence
DLVMTQSPSSLSASVGDRVTITCKSSQSLFNDKTRKNYLAWYQQKPGKAPKLLIYWASTRESGVPARFSGSGSGTDFTLTISSLEPEDFAVYYCQQEYYQPYTFGQGTKVEIK

SEQ ID NO: 106 - protein, 113 aa, Artificial Sequence
  An artificially synthesized sequence
DLVMTQSPSSLSASVGDRVTITCKSSQSLFNDKTRKNYLAWYQQKPGKAPKLLIYWASTRESGVPARFSGSGSGTDFTLTISSLEPEDFAVYYCQQFYYQPYTFGQGTKVEIK

SEQ ID NO: 107 - protein, 113 aa, Artificial Sequence
  An artificially synthesized sequence
DLVMTQSPSSLSASVGDRVTITCKSSQSLFNDKTRKNYLAWYQQKPGKAPKLLIYWASTRESGVPARFSGSGSGTDFTLTISSLEPEDFAVYYCQQEYYQMYTFGQGTKVEIK

SEQ ID NO: 108 - protein, 113 aa, Artificial Sequence
  An artificially synthesized sequence
DLVMTQSPSSLSASVGDRVTITCKSSQSLFNDKTRKNYLAWYQQKPGKAPKLLIYWASTRESGVPARFSGSGSGTDFTLTISSLEPEDFAVYYCQQFYYQMYTFGQGTKVEIK

SEQ ID NO: 109 - protein, 113 aa, Artificial Sequence
  An artificially synthesized sequence
DLVMTQSPSSLSASVGDRVTITCKSSQSLFNEKTRKNYLAWYQQKPGKAPKLLIYWASTRESGVPARFSGSGSGTDFTLTISSLEPEDFAVYYCQQEYYEPYTFGQGTKVEIK

SEQ ID NO: 110 - protein, 113 aa, Artificial Sequence
  An artificially synthesized sequence
DLVMTQSPSSLSASVGDRVTITCKSSQSLFNEKTRKNYLAWYQQKPGKAPKLLIYWASTRESGVPARFSGSGSGTDFTLTISSLEPEDFAVYYCQQFYYEPYTFGQGTKVEIK

SEQ ID NO: 111 - protein, 113 aa, Artificial Sequence
  An artificially synthesized sequence
DLVMTQSPSSLSASVGDRVTITCKSSQSLFNEKTRKNYLAWYQQKPGKAPKLLIYWASTRESGVPARFSGSGSGTDFTLTISSLEPEDFAVYYCQQEYYQPYTFGQGTKVEIK

SEQ ID NO: 112 - protein, 113 aa, Artificial Sequence
  An artificially synthesized sequence
DLVMTQSPSSLSASVGDRVTITCKSSQSLFNEKTRKNYLAWYQQKPGKAPKLLIYWASTRESGVPARFSGSGSGTDFTLTISSLEPEDFAVYYCQQFYYQPYTFGQGTKVEIK

SEQ ID NO: 113 - protein, 113 aa, Artificial Sequence
  An artificially synthesized sequence
DLVMTQSPSSLSASVGDRVTITCKSSQSLFNSSTSKNYLAWYQQKPGKAPKLLIYWASTRESGVPARFSGSGSGTDFTLTISSLEPEDFAVYYCHQYYYEPYTFGQGTKVEIK

SEQ ID NO: 114 - protein, 113 aa, Artificial Sequence
  An artificially synthesized sequence
DLVMTQSPSSLSASVGDRVTITCKSSQSLFNSSTSKNYLAWYQQKPGKAPKLLIYWASTRESGVPARFSGSGSGTDFTLTISSLEPEDFAVYYCYQYYYEPYTFGQGTKVEIK

SEQ ID NO: 115 - protein, 113 aa, Artificial Sequence
  An artificially synthesized sequence
DLVMTQSPSSLSASVGDRVTITCKSSQSLFNSSTSKNYLAWYQQKPGKAPKLLIYWASTRESGVPARFSGSGSGTDFTLTISSLEPEDFAVYYCQQEYYEPYTFGQGTKVEIK

SEQ ID NO: 116 - protein, 113 aa, Artificial Sequence
  An artificially synthesized sequence
DLVMTQSPSSLSASVGDRVTITCKSSQSLFNSSTSKNYLAWYQQKPGKAPKLLIYWASTRESGVPARFSGSGSGTDFTLTISSLEPEDFAVYYCQQFYYEPYTFGQGTKVEIK

SEQ ID NO: 117 - protein, 113 aa, Artificial Sequence
  An artificially synthesized sequence
DLVMTQSPSSLSASVGDRVTITCKSSQSLFNSSTSKNYLAWYQQKPGKAPKLLIYWASTRESGVPARFSGSGSGTDFTLTISSLEPEDFAVYYCHQYYYQPYTFGQGTKVEIK

SEQ ID NO: 118 - protein, 113 aa, Artificial Sequence
  An artificially synthesized sequence
DLVMTQSPSSLSASVGDRVTITCKSSQSLFNSSTSKNYLAWYQQKPGKAPKLLIYWASTRESGVPARFSGSGSGTDFTLTISSLEPEDFAVYYCYQYYYQPYTFGQGTKVEIK

SEQ ID NO: 119 - protein, 113 aa, Artificial Sequence
  An artificially synthesized sequence
DLVMTQSPSSLSASVGDRVTITCKSSQSLFNSSTSKNYLAWYQQKPGKAPKLLIYWASTRESGVPARFSGSGSGTDFTLTISSLEPEDFAVYYCQQEYYQPYTFGQGTKVEIK

SEQ ID NO: 120 - protein, 113 aa, Artificial Sequence
  An artificially synthesized sequence
DLVMTQSPSSLSASVGDRVTITCKSSQSLFNSSTSKNYLAWYQQKPGKAPKLLIYWASTRESGVPARFSGSGSGTDFTLTISSLEPEDFAVYYCQQFYYQPYTFGQGTKVEIK

SEQ ID NO: 121 - protein, 113 aa, Artificial Sequence
  An artificially synthesized sequence
DLVMTQSPSSLSASVGDRVTITCKSSQSLFESSTSKNYLAWYQQKPGKAPKLLIYWASTRESGVPARFSGSGSGTDFTLTISSLEPEDFAVYYCYQYYYQPYTFGQGTKVEIK

SEQ ID NO: 122 - protein, 113 aa, Artificial Sequence
  An artificially synthesized sequence
DLVMTQSPSSLSASVGDRVTITCKSSQSLFESSTSKNYLAWYQQKPGKAPKLLIYWASTRESGVPARFSGSGSGTDFTLTISSLEPEDFAVYYCQQFYYQPYTFGQGTKVEIK

SEQ ID NO: 123 - protein, 113 aa, Artificial Sequence
  An artificially synthesized sequence
DIQMTQSPSSLSASVGDRVTITCKSSQSLFESKTRKNYLAWYQQKPGKAPKLLIYWASTRESGVPARFSGSGSGTDFTLTISSLEPEDFAVYYCHQYYYEPYTFGQGTKVEIK

SEQ ID NO: 124 - protein, 113 aa, Artificial Sequence
  An artificially synthesized sequence
DIQMTQSPSSLSASVGDRVTITCKSSQSLFESKTRKNYLAWYQQKPGKAPKLLIYWASTRESGVPARFSGSGSGTDFTLTISSLEPEDFAVYYCYQYYYEPYTFGQGTKVEIK

SEQ ID NO: 125 - protein, 113 aa, Artificial Sequence
  An artificially synthesized sequence
DIQMTQSPSSLSASVGDRVTITCKSSQSLFESKTRKNYLAWYQQKPGKAPKLLIYWASTRESGVPARFSGSGSGTDFTLTISSLEPEDFAVYYCQQEYYEPYTFGQGTKVEIK

SEQ ID NO: 126 - protein, 113 aa, Artificial Sequence
  An artificially synthesized sequence
DIQMTQSPSSLSASVGDRVTITCKSSQSLFESKTRKNYLAWYQQKPGKAPKLLIYWASTRESGVPARFSGSGSGTDFTLTISSLEPEDFAVYYCQQFYYEPYTFGQGTKVEIK

SEQ ID NO: 127 - protein, 113 aa, Artificial Sequence
  An artificially synthesized sequence
DIQMTQSPSSLSASVGDRVTITCKSSQSLFESSTRKNYLAWYQQKPGKAPKLLIYWASTRESGVPARFSGSGSGTDFTLTISSLEPEDFAVYYCHQYYYEPYTFGQGTKVEIK

SEQ ID NO: 128 - protein, 113 aa, Artificial Sequence
  An artificially synthesized sequence
DIQMTQSPSSLSASVGDRVTITCKSSQSLFESSTRKNYLAWYQQKPGKAPKLLIYWASTRESGVPARFSGSGSGTDFTLTISSLEPEDFAVYYCYQYYYEPYTFGQGTKVEIK

SEQ ID NO: 129 - protein, 113 aa, Artificial Sequence
  An artificially synthesized sequence
DIQMTQSPSSLSASVGDRVTITCKSSQSLFESSTRKNYLAWYQQKPGKAPKLLIYWASTRESGVPARFSGSGSGTDFTLTISSLEPEDFAVYYCQQFYYEPYTFGQGTKVEIK

SEQ ID NO: 130 - protein, 113 aa, Artificial Sequence
  An artificially synthesized sequence
DIQMTQSPSSLSASVGDRVTITCKSSQSLFESSTRKNYLAWYQQKPGKAPKLLIYWASTRESGVPARFSGSGSGTDFTLTISSLEPEDFAVYYCHQYYYQPYTFGQGTKVEIK

SEQ ID NO: 131 - protein, 113 aa, Artificial Sequence
  An artificially synthesized sequence
DIQMTQSPSSLSASVGDRVTITCKSSQSLFESSTRKNYLAWYQQKPGKAPKLLIYWASTRESGVPARFSGSGSGTDFTLTISSLEPEDFAVYYCYQYYYQPYTFGQGTKVEIK

SEQ ID NO: 132 - protein, 113 aa, Artificial Sequence
  An artificially synthesized sequence
DIQMTQSPSSLSASVGDRVTITCKSSQSLFESSTRKNYLAWYQQKPGKAPKLLIYWASTRESGVPARFSGSGSGTDFTLTISSLEPEDFAVYYCQQEYYQPYTFGQGTKVEIK

SEQ ID NO: 133 - protein, 113 aa, Artificial Sequence
  An artificially synthesized sequence
DIQMTQSPSSLSASVGDRVTITCKSSQSLFESSTRKNYLAWYQQKPGKAPKLLIYWASTRESGVPARFSGSGSGTDFTLTISSLEPEDFAVYYCQQFYYQPYTFGQGTKVEIK

SEQ ID NO: 134 - protein, 113 aa, Artificial Sequence
  An artificially synthesized sequence
DIQMTQSPSSLSASVGDRVTITCKSSQSLFESVTRKNYLAWYQQKPGKAPKLLIYWASTRESGVPARFSGSGSGTDFTLTISSLEPEDFAVYYCYQYYYEPYTFGQGTKVEIK

SEQ ID NO: 135 - protein, 113 aa, Artificial Sequence
  An artificially synthesized sequence
DIQMTQSPSSLSASVGDRVTITCKSSQSLFESVTRKNYLAWYQQKPGKAPKLLIYWASTRESGVPARFSGSGSGTDFTLTISSLEPEDFAVYYCQQFYYEPYTFGQGTKVEIK

SEQ ID NO: 136 - protein, 113 aa, Artificial Sequence
  An artificially synthesized sequence
DIQMTQSPSSLSASVGDRVTITCKSSQSLFESRTSKNYLAWYQQKPGKAPKLLIYWASTRESGVPARFSGSGSGTDFTLTISSLEPEDFAVYYCHQYYYEPYTFGQGTKVEIK

SEQ ID NO: 137 - protein, 113 aa, Artificial Sequence
  An artificially synthesized sequence
DIQMTQSPSSLSASVGDRVTITCKSSQSLFESRTSKNYLAWYQQKPGKAPKLLIYWASTRESGVPARFSGSGSGTDFTLTISSLEPEDFAVYYCYQYYYEPYTFGQGTKVEIK

SEQ ID NO: 138 - protein, 113 aa, Artificial Sequence
  An artificially synthesized sequence
DIQMTQSPSSLSASVGDRVTITCKSSQSLFESRTRLNYLAWYQQKPGKAPKLLIYWASTRESGVPARFSGSGSGTDFTLTISSLEPEDFAVYYCHQYYYEPYTFGQGTKVEIK

SEQ ID NO: 139 - protein, 113 aa, Artificial Sequence
  An artificially synthesized sequence
DIQMTQSPSSLSASVGDRVTITCKSSQSLFESRTRLNYLAWYQQKPGKAPKLLIYWASTRESGVPARFSGSGSGTDFTLTISSLEPEDFAVYYCYQYYYEPYTFGQGTKVEIK

SEQ ID NO: 140 - protein, 113 aa, Artificial Sequence
  An artificially synthesized sequence
DIQMTQSPSSLSASVGDRVTITCKSSQSLFNDKTRKNYLAWYQQKPGKAPKLLIYWASTRESGVPARFSGSGSGTDFTLTISSLEPEDFAVYYCQQEYYEPYTFGQGTKVEIK

SEQ ID NO: 141 - protein, 113 aa, Artificial Sequence
  An artificially synthesized sequence
DIQMTQSPSSLSASVGDRVTITCKSSQSLFNDKTRKNYLAWYQQKPGKAPKLLIYWASTRESGVPARFSGSGSGTDFTLTISSLEPEDFAVYYCQQFYYEPYTFGQGTKVEIK

SEQ ID NO: 142 - protein, 113 aa, Artificial Sequence
  An artificially synthesized sequence
DIQMTQSPSSLSASVGDRVTITCKSSQSLFNDKTRKNYLAWYQQKPGKAPKLLIYWASTRESGVPARFSGSGSGTDFTLTISSLEPEDFAVYYCQQEYYQPYTFGQGTKVEIK

SEQ ID NO: 143 - protein, 113 aa, Artificial Sequence
  An artificially synthesized sequence
DIQMTQSPSSLSASVGDRVTITCKSSQSLFNDKTRKNYLAWYQQKPGKAPKLLIYWASTRESGVPARFSGSGSGTDFTLTISSLEPEDFAVYYCQQFYYQPYTFGQGTKVEIK

SEQ ID NO: 144 - protein, 113 aa, Artificial Sequence
  An artificially synthesized sequence
DIQMTQSPSSLSASVGDRVTITCKSSQSLFNDKTRKNYLAWYQQKPGKAPKLLIYWASTRESGVPARFSGSGSGTDFTLTISSLEPEDFAVYYCQQEYYQMYTFGQGTKVEIK

SEQ ID NO: 145 - protein, 113 aa, Artificial Sequence
  An artificially synthesized sequence
DIQMTQSPSSLSASVGDRVTITCKSSQSLFNDKTRKNYLAWYQQKPGKAPKLLIYWASTRESGVPARFSGSGSGTDFTLTISSLEPEDFAVYYCQQFYYQMYTFGQGTKVEIK

SEQ ID NO: 146 - protein, 113 aa, Artificial Sequence
  An artificially synthesized sequence
DIQMTQSPSSLSASVGDRVTITCKSSQSLFNEKTRKNYLAWYQQKPGKAPKLLIYWASTRESGVPARFSGSGSGTDFTLTISSLEPEDFAVYYCQQEYYEPYTFGQGTKVEIK

SEQ ID NO: 147 - protein, 113 aa, Artificial Sequence
  An artificially synthesized sequence
DIQMTQSPSSLSASVGDRVTITCKSSQSLFNEKTRKNYLAWYQQKPGKAPKLLIYWASTRESGVPARFSGSGSGTDFTLTISSLEPEDFAVYYCQQFYYEPYTFGQGTKVEIK

SEQ ID NO: 148 - protein, 113 aa, Artificial Sequence
  An artificially synthesized sequence
DIQMTQSPSSLSASVGDRVTITCKSSQSLFNEKTRKNYLAWYQQKPGKAPKLLIYWASTRESGVPARFSGSGSGTDFTLTISSLEPEDFAVYYCQQEYYQPYTFGQGTKVEIK

SEQ ID NO: 149 - protein, 113 aa, Artificial Sequence
  An artificially synthesized sequence
DIQMTQSPSSLSASVGDRVTITCKSSQSLFNEKTRKNYLAWYQQKPGKAPKLLIYWASTRESGVPARFSGSGSGTDFTLTISSLEPEDFAVYYCQQFYYQPYTFGQGTKVEIK

SEQ ID NO: 150 - protein, 113 aa, Artificial Sequence
  An artificially synthesized sequence
DIQMTQSPSSLSASVGDRVTITCKSSQSLFESSTSKNYLAWYQQKPGKAPKLLIYWASTRESGVPARFSGSGSGTDFTLTISSLEPEDFAVYYCYQYYYQPYTFGQGTKVEIK

SEQ ID NO: 151 - protein, 128 aa, Artificial Sequence
  An artificially synthesized sequence
QVQLVESGGGLVQPGGSLTLSCAASGFPLDYYAIGWFRQAPGKEREGVSSISSSDGSTYYADSVKGRFTISRGNAKNTVYLQMNSLKPEDTAVYYCAAVHTTARAMGVPKWAYEYDYWGQGTQVTVSS

SEQ ID NO: 152 - protein, 119 aa, Artificial Sequence [sequence omitted]

SEQ ID NO: 153 - protein, 107 aa, Artificial Sequence [sequence omitted]

SEQ ID NO: 154 - protein, 328 aa, Artificial Sequence
  An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 155 - protein, 328 aa, Artificial Sequence
  An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 156 - protein, 108 aa, Artificial Sequence
  An artificially synthesized sequence
AVVLTQTASPVSAPVGGTVTIKCQASESIGNALAWYQQKPGQPPKLLIYLGSTLASGVPSRFSGSGSGTEFTLTISDLECADVGTYYCQKIVGTSSMTFGGGTKVEIK

SEQ ID NO: 157 - protein, 107 aa, Artificial Sequence
  An artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVK

SEQ ID NO: 158 - protein, 120 aa, Artificial Sequence
  An artificially synthesized sequence
EVQLVESGGGLVQPGGSLRLSCAASGYTFTSYVMHWVRQAPGKGLEWVSAINPYNDGNYYADSVKGRFTISRDNSRKTLYLQMNSLRAEDTAVYYCARGAYKRGYAMDYWGQGTTVTVSS

SEQ ID NO: 159 - protein, 119 aa, Artificial Sequence
  An artificially synthesized sequence
QSVEESGGRLVTPGTPLTLTCTVSGFSLSNYAMSWVRQAPGKGLQYIGIIYTGGSAYYASWAKGRFTISRTSTTVDLKMTSLTTEDTATYFCARDFASSSGYYGYFNLWGPGTLVTVSS

SEQ ID NO: 160 - protein, 116 aa, Artificial Sequence
  An artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 161 - protein, 113 aa, Artificial Sequence
  An artificially synthesized sequence
DIQMTQSPSSLSASVGDRVTITCKSSQSLFNDKTRKNYLAWYQQKPGKAPKLLIYAASTRESGVPARFSGSGSGTDFTLTISSLEPEDFAVYYCQQFYYQMYTFGQGTKVEIK

SEQ ID NO: 162 - protein, 113 aa, Artificial Sequence
  An artificially synthesized sequence
DIQMTQSPSSLSASVGDRVTITCKSSQSLFNDKTRKNYLAWYQQKPGKAPKLLIYGASTRESGVPARFSGSGSGTDFTLTISSLEPEDFAVYYCQQFYYQMYTFGQGTKVEIK

SEQ ID NO: 163 - protein, 113 aa, Artificial Sequence
  An artificially synthesized sequence
DIQMTQSPSSLSASVGDRVTITCKSSQSLFNDKTRKNYLAWYQQKPGKAPKLLIYIASTRESGVPARFSGSGSGTDFTLTISSLEPEDFAVYYCQQFYYQMYTFGQGTKVEIK

SEQ ID NO: 164 - protein, 113 aa, Artificial Sequence
  An artificially synthesized sequence
DIQMTQSPSSLSASVGDRVTITCKSSQSLFNDKTRKNYLAWYQQKPGKAPKLLIYTASTRESGVPARFSGSGSGTDFTLTISSLEPEDFAVYYCQQFYYQMYTFGQGTKVEIK

SEQ ID NO: 165 - protein, 113 aa, Artificial Sequence
  An artificially synthesized sequence
DIQMTQSPSSLSASVGDRVTITCKSSQSLFNDKTRKNYLAWYQQKPGKAPKLLIYVASTRESGVPARFSGSGSGTDFTLTISSLEPEDFAVYYCQQFYYQMYTFGQGTKVEIK

SEQ ID NO: 166 - protein, 113 aa, Artificial Sequence
  An artificially synthesized sequence
DIQMTQSPSSLSASVGDRVTITCKSSQSLFNDKTRKNYLAWYQQKPGKAPKLLIYWAFTRESGVPARFSGSGSGTDFTLTISSLEPEDFAVYYCQQFYYQMYTFGQGTKVEIK

SEQ ID NO: 167 - protein, 113 aa, Artificial Sequence
  An artificially synthesized sequence
DIQMTQSPSSLSASVGDRVTITCKSSQSLFNDKTRKNYLAWYQQKPGKAPKLLIYWAITRESGVPARFSGSGSGTDFTLTISSLEPEDFAVYYCQQFYYQMYTFGQGTKVEIK

SEQ ID NO: 168 - protein, 113 aa, Artificial Sequence
  An artificially synthesized sequence
DIQMTQSPSSLSASVGDRVTITCKSSQSLFNDKTRKNYLAWYQQKPGKAPKLLIYWASNRESGVPARFSGSGSGTDFTLTISSLEPEDFAVYYCQQFYYQMYTFGQGTKVEIK

SEQ ID NO: 169 - protein, 113 aa, Artificial Sequence
  An artificially synthesized sequence
DIQMTQSPSSLSASVGDRVTITCKSSQSLFNDKTRKNYLAWYQQKPGKAPKLLIYWASYRESGVPARFSGSGSGTDFTLTISSLEPEDFAVYYCQQFYYQMYTFGQGTKVEIK

SEQ ID NO: 170 - protein, 113 aa, Artificial Sequence
  An artificially synthesized sequence
DIQMTQSPSSLSASVGDRVTITCKSSQSLFNDKTRKNYLAWYQQKPGKAPKLLIYWASTKESGVPARFSGSGSGTDFTLTISSLEPEDFAVYYCQQFYYQMYTFGQGTKVEIK

SEQ ID NO: 171 - protein, 113 aa, Artificial Sequence
  An artificially synthesized sequence
DIQMTQSPSSLSASVGDRVTITCKSSQSLFNDKTRKNYLAWYQQKPGKAPKLLIYIAFTRESGVPARFSGSGSGTDFTLTISSLEPEDFAVYYCQQFYYQMYTFGQGTKVEIK

SEQ ID NO: 172 - protein, 113 aa, Artificial Sequence
  An artificially synthesized sequence
DIQMTQSPSSLSASVGDRVTITCKSSQSLFNDKTRKNYLAWYQQKPGKAPKLLIYIASNRESGVPARFSGSGSGTDFTLTISSLEPEDFAVYYCQQFYYQMYTFGQGTKVEIK

SEQ ID NO: 173 - protein, 113 aa, Artificial Sequence
  An artificially synthesized sequence
DIQMTQSPSSLSASVGDRVTITCKSSQSLFNDKTRKNYLAWYQQKPGKAPKLLIYIASTVESGVPARFSGSGSGTDFTLTISSLEPEDFAVYYCQQFYYQMYTFGQGTKVEIK

SEQ ID NO: 174 - protein, 113 aa, Artificial Sequence
  An artificially synthesized sequence
DLVMTQSPSSLSASVGDRVTITCKSSQSLFNSRTRKNYLAWYQQKPGKAPKLLIYWASTRESGVPSRFSGSGSGTDFTLTISSLQPEDFATYYCQQYYYHMYTFGQGTKVEIK

SEQ ID NO: 175 - protein, 4 aa, Artificial Sequence
  An artificially synthesized sequence
GGGS

SEQ ID NO: 176 - protein, 4 aa, Artificial Sequence
  An artificially synthesized sequence
GGSG

SEQ ID NO: 177 - protein, 4 aa, Artificial Sequence
  An artificially synthesized sequence
GSGG

SEQ ID NO: 178 - protein, 4 aa, Artificial Sequence
  An artificially synthesized sequence
SGGG

SEQ ID NO: 179 - protein, 4 aa, Artificial Sequence
  An artificially synthesized sequence
GSSG

SEQ ID NO: 180 - protein, 5 aa, Artificial Sequence
  An artificially synthesized sequence
GGGGS

SEQ ID NO: 181 - protein, 5 aa, Artificial Sequence
  An artificially synthesized sequence
GGGSG

SEQ ID NO: 182 - protein, 5 aa, Artificial Sequence
  An artificially synthesized sequence
GGSGG

SEQ ID NO: 183 - protein, 5 aa, Artificial Sequence
  An artificially synthesized sequence
GSGGG

SEQ ID NO: 184 - protein, 5 aa, Artificial Sequence
  An artificially synthesized sequence
GSGGS

SEQ ID NO: 185 - protein, 5 aa, Artificial Sequence
  An artificially synthesized sequence
SGGGG

SEQ ID NO: 186 - protein, 5 aa, Artificial Sequence
  An artificially synthesized sequence
GSSGG

SEQ ID NO: 187 - protein, 5 aa, Artificial Sequence
  An artificially synthesized sequence
GSGSG

SEQ ID NO: 188 - protein, 5 aa, Artificial Sequence
  An artificially synthesized sequence
SGGSG

SEQ ID NO: 189 - protein, 5 aa, Artificial Sequence
  An artificially synthesized sequence
GSSSG

SEQ ID NO: 190 - protein, 6 aa, Artificial Sequence
  An artificially synthesized sequence
GGGGGS

SEQ ID NO: 191 - protein, 6 aa, Artificial Sequence
  An artificially synthesized sequence
SGGGGG

SEQ ID NO: 192 - protein, 7 aa, Artificial Sequence
  An artificially synthesized sequence
GGGGGGS

SEQ ID NO: 193 - protein, 7 aa, Artificial Sequence
  An artificially synthesized sequence
SGGGGGG

SEQ ID NO: 194 - protein, 5 aa, Artificial Sequence
  An artificially synthesized sequence
GGGGS

SEQ ID NO: 195 - protein, 5 aa, Artificial Sequence
  An artificially synthesized sequence
SGGGG

SEQ ID NO: 196 - protein, 433 aa, Artificial Sequence
  An artificially synthesized sequence
MAIQFRSLFPLALPGMLALLGWWWFFSRKKSRVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKDISGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 197 - protein, 30 aa, Artificial Sequence
  An artificially synthesized sequence
MAIQFRSLFPLALPGMLALLGWWWFFSRKK

SEQ ID NO: 198 - protein, 299 aa, Artificial Sequence
  An artificially synthesized sequence
MDYKDDDDKTKEYQDLQHLDNEESDHHQLRKGPPPPQPLLQRLCSGPRLLLLSLGLSLLLLVVVCVIGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 199 - protein, 312 aa, Artificial Sequence
  An artificially synthesized sequence
MHHHHHHAKDFQDIQQLSSEENDHPFHQGEGPGTRRLNPRRGNPFLKGPPPAQPLAQRLCSMVCFSLLALSFNILLLVVICVTGSQSAQLQAELRSLKEAFSNFSSSTLTEVQAISTHGGSVGDKITSLGAKLEKQQQDLKADHDALLFHLKHFPVDLRFVACQMELLHSNGSQRTCCPVNWVEHQGSCYWFSHSGKAWAEAEKYCQLENAHLVVINSWEEQKFIVQHTNPFNTWIGLTDSDGSWKWVDGTDYRHNYKNWAVTQPDNWHGHELGGSEDCVEVQPDGRWNDDFCLQVYRWVCEKRRNATGEVA

SEQ ID NO: 200 - protein, 402 aa, Artificial Sequence
  An artificially synthesized sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKDISGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 201 - protein, 321 aa, Artificial Sequence
  An artificially synthesized sequence
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 202 - protein, 11 aa, Artificial Sequence
  An artificially synthesized sequence
VTGYRLFEEIL

SEQ ID NO: 203 - protein, 158 aa, Artificial Sequence
  An artificially synthesized sequence
VFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS